SEQUENCE LISTING (41 sequences)

SEQ ID NO: 1 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 2 - protein, 327 aa, Homo sapiens
MLFLLLPLLAVLPGDGNADGLKEPLSFHVTWIASFYNHSWKQNLVSGWLSDLQTHTWDSNSSTIVFLCPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEHHSSVGFIILAVIVPLLLLIGLALWFRKRCFC

SEQ ID NO: 3 - protein, 335 aa, Homo sapiens
MGCLLFLLLWALLQAWGSAEVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSMGLIALAVLACLLFLLIVGFTSRFKRQTSYQGVL

SEQ ID NO: 4 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 5 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 6 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 7 - protein, 178 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 8 - protein, 1033 aa, Homo sapiens
MGAAGLLGVFLALVAPGVLGISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEIFCPSPPPILNGRHIGNSLANVSYGSIVTYTCDPDPEEGVNFILIGESTLRCTVDSQKTGTWSGPAPRCELSTSAVQCPHPQILRGRMVSGQKDRYTYNDTVIFACMFGFTLKGSKQIRCNAQGTWEPSAPVCEKECQAPPNILNGQKEDRHMVRFDPGTSIKYSCNPGYVLVGEESIQCTSEGVWTPPVPQCKVAACEATGRQLLTKPQHQFVRPDVNSSCGEGYKLSGSVYQECQGTIPWFMEIRLCKEITCPPPPVIYNGAHTGSSLEDFPYGTTVTYTCNPGPERGVEFSLIGESTIRCTSNDQERGTWSGPAPLCKLSLLAVQCSHVHIANGYKISGKEAPYFYNDTVTFKCYSGFTLKGSSQIRCKADNTWDPEIPVCEKETCQHVRQSLQELPAGSRVELVNTSCQDGYQLTGHAYQMCQDAENGIWFKKIPLCKVIHCHPPPVIVNGKHTGMMAENFLYGNEVSYECDQGFYLLGEKKLQCRSDSKGHGSWSGPSPQCLRSPPVTRCPNPEVKHGYKLNKTHSAYSHNDIVYVDCNPGFIMNGSRVIRCHTDNTWVPGVPTCIKKAFIGCPPPPKTPNGNHTGGNIARFSPGMSILYSCDQGYLLVGEALLLCTHEGTWSQPAPHCKEVNCSSPADMDGIQKGLEPRKMYQYGAVVTLECEDGYMLEGSPQSQCQSDHQWNPPLAVCRSRSLAPVLCGIAAGLILLTFLIVITLYVISKHRARNYYTDTSQKEAFHLEAREVYSVDPYNPAS

SEQ ID NO: 9 - protein, 369 aa, Homo sapiens
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 10 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 11 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 12 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 13 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 14 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 15 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 16 - protein, 448 aa, Homo sapiens
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 17 - protein, 453 aa, Homo sapiens
MQGAQEASASEMLPLLLPLLWAGALAQERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVLGAVWGASITTLVFLCVCFIFRVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 18 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 19 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 20 - protein, 556 aa, Homo sapiens [sequence omitted]

SEQ ID NO: 21 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 22 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 23 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 24 - protein, 1136 aa, Homo sapiens
MFSRKKRELMKTPSISKKNRAGSPSPQPSGELPRKDGADAVFPGPSLEPPAGSSGVKATGTLKRPTSLSRHASAAGFPLSGAASWTLGRSHRSPLTAASPGELPTEGAGPDVVEDISHLLADVARFAEGLEKLKECVLRDDLLEARRPRAHECLGEALRVMHQIISKYPLLNTVETLTAAGTLIAKVKAFHYESNNDLEKQEFEKALETIAVAFSSTVSEFLMGEVDSSTLLAVPPGDSSQSMESLYGPGSEGTPPSLEDCDAGCLPAEEVDVLLQRCEGGVDAALLYAKNMAKYMKDLISYLEKRTTLEMEFAKGLQKIAHNCRQSVMQEPHMPLLSIYSLALEQDLEFGHSMVQAVGTLQTQTFMQPLTLRRLEHEKRRKEIKEAWHRAQRKLQEAESNLRKAKQGYVQRCEDHDKARFLVAKAEEEQAGSAPGAGSTATKTLDKRRRLEEEAKNKAEEAMATYRTCVADAKTQKQELEDTKVTALRQIQEVIRQSDQTIKSATISYYQMMHMQTAPLPVHFQMLCESSKLYDPGQQYASHVRQLQRDQEPDVHYDFEPHVSANAWSPVMRARKSSFNVSDVARPEAAGSPPEEGGCTEGTPAKDHRAGRGHQVHKSWPLSISDSDSGLDPGPGAGDFKKFERTSSSGTMSSTEELVDPDGGAGASAFEQADLNGMTPELPVAVPSGPFRHEGLSKAARTHRLRKLRTPAKCRECNSYVYFQGAECEECCLACHKKCLETLAIQCGHKKLQGRLQLFGQDFSHAARSAPDGVPFIVKKCVCEIERRALRTKGIYRVNGVKTRVEKLCQAFENGKELVELSQASPHDISNVLKLYLRQLPEPLISFRLYHELVGLAKDSLKAEAEAKAASRGRQDGSESEAVAVALAGRLRELLRDLPPENRASLQYLLRHLRRIVEVEQDNKMTPGNLGIVFGPTLLRPRPTEATVSLSSLVDYPHQARVIETLIVHYGLVFEEEPEETPGGQDESSNQRAEVVVQVPYLEAGEAVVYPLQEAAADGCRESRVVSNDSDSDLEEASELLSSSEASALGHLSFLEQQQSEASLEVASGSHSGSEEQLEATAREDGDGDEDGPAQQLSGFNTNQSNNVLQAPLPPMRLRGGRMTLGSCRERQPEFV

SEQ ID NO: 25 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 26 - protein, 9 aa, Synthetic construct
RMFPNAPYL

SEQ ID NO: 27 - protein, 9 aa, Synthetic construct
CMTWNQMNL

SEQ ID NO: 28 - protein, 9 aa, Synthetic construct
VLDGLDVLL

SEQ ID NO: 29 - protein, 10 aa, Synthetic construct
ALYVDSLEFL

SEQ ID NO: 30 - protein, 10 aa, Synthetic construct
SLYSFPEPEA

SEQ ID NO: 31 - protein, 9 aa, Synthetic construct
SLLQHLIGL

SEQ ID NO: 32 - protein, 10 aa, Synthetic construct
LYVDSLFFLC

SEQ ID NO: 33 - protein, 9 aa, Synthetic construct
VLHDDLLEA

SEQ ID NO: 34 - protein, 9 aa, Synthetic construct
CLAVEEVSL

SEQ ID NO: 35 - protein, 16 aa, Synthetic construct
KRYFKLSHLQMHSRKH

SEQ ID NO: 36 - protein, 118 aa, Synthetic construct
GAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 37 - protein, 107 aa, Synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 38 - protein, 107 aa, Synthetic construct
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 39 - protein, 121 aa, Synthetic construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASGA

SEQ ID NO: 40 - protein, 120 aa, Synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 41 - protein, 118 aa, Synthetic construct
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

(19) United States
(12) Patent Application Publication
(10) Pub. No.: US 2025/0170240 A1
(43) Pub. Date: May 29, 2025

(54) MODIFIED INVARIANT NATURAL KILLER T CELLS EXPRESSING A CHIMERIC ANTIGEN RECEPTOR AND USES THEREOF

(71) Applicant: Tinkeso Therapeutics, Inc., Valhalla, NY (US)

(72) Inventors: Xianzheng Zhou, Yorktown Heights, NY (US); Xin Huang, Yorktown Heights, NY (US)

(21) Appl. No.: 18/840,585

(22) PCT Filed: Feb. 22, 2023
(86) PCT No.: PCT/US23/63019

§ 371 Date: Aug. 22, 2024

Related U.S. Application Data
(60) Provisional application No. 63/313,186, filed on Feb. 23, 2022.

(51) Int. Cl. A61K 40/15; A61K 40/31; A61K 40/42; A61P 35/02; C07K 16/28; C12N 5/0783

(52) U.S. Cl. CPC A61K 40/15; A61K 40/31; A61K 40/421; A61P 35/02; C07K 16/2803; C12N 5/0646; C07K 2317/53; C07K 2319/02; C07K 2319/03; C12N 2500/36; C12N 2501/2302; C12N 2501/2307; C12N 2501/2315; C12N 2510/00

(57) ABSTRACT

The present disclosure relates to modified invariant natural killer T (INKT) cells or type I natural killer T (NKT) cells (e.g., CD3⁺iTCR Vα24-Jα18⁺ or CD3⁺Vα24⁺) to express a CD7 chimeric antigen receptor (CAR) as therapeutic agents for the treatment of a CD7 cancer, and pharmaceutical compositions, methods of preparation, and therapeutic uses thereof.

### CROSS-REFERENCE TO RELATED APPLICATION

This application claims priority under 35 U.S.C. § 119 (e) to U.S. Provisional Patent Application No. 63/313,186, filed on Feb. 23, 2022, the disclosure of which is incorporated herein by reference in its entirety.

### FIELD OF THE DISCLOSURE

The present disclosure relates to modified invariant natural killer T (NKT) cells (e.g., INKT cells, type I NKT cells) expressing a CD7 chimeric antigen receptor, pharmaceutical compositions, methods of preparation, and therapeutic use of the cells or pharmaceutical compositions thereof for treatment or prevention of a CD7⁺ cancer.

### BACKGROUND OF THE DISCLOSURE

Natural killer T (NKT) cells are a T-cell subset that exhibits characteristics of both conventional T cells and natural killer (NK) cells. NKT cells typically arise in the thymus from CD4⁺CD8⁺ cortical thymocytes that have undergone T cell receptor (TCR) gene rearrangement. NKT cells have been traditionally defined as CD1d-restricted, lipid antigen-reactive T cells and classified as type I and type II NKT cells (Godfrey et al., Immunity 2018; 48 (3): 453-73; Dhodapkar and Kumar, J Immunol. 2017; 198 (3): 1015-21). Based on their TCR repertoire, antigen specificity and CD1d dependence, NKT cells have also been divided into three categories: type I, type II, and type III NKT cells (Godfrey et al., Nat Rev Immunol. 2004; 4 (3): 231-37).

Type I or invariant NKT (INKT) cells express an invariant TCRα-chain (Vα14-Jα18 in mice and Vα24-Jα18 in humans) and a limited number of non-invariant TCRβ-chains (Vβ8.2, Vβ7, and Vβ2 in mice, Vβ11 in humans) (Godfrey et al., Nat Immunol. 2010; 11 (3): 197-206; Krovi and Gapin., Front Immunol. 2018; 9 (6): 1939). INKT (type I NKT, interchangeably used throughout) cells also recognize the glycosphingolipid α-galactosylceramide (αGalCer) antigen when presented by major histocompatibility complex (MHC) class I-like CD1d molecules (Kawano et al., Science. 1997; 278 (5343): 1626-1629). Type II NKT cells have a more diverse and less well-defined TCR repertoire and recognize non-αGalCer molecules (such as sulfatide) presented by CD1d molecules. Type III NKT or NKT-like cells have a diverse TCR repertoire and recognize CD1d-independent molecules.

The use of INKT cells co-expressing chimeric antigen receptors (CAR-INKT) with interleukin-15 (IL-15) or allogeneic hematopoietic stem cells engineered INKT (HSC-INKT) or single-chain bispecific antibody that stabilizes an invariant T cell receptor (iTCR)-CD1d complex has recently generated promising anti-tumor results in patients with neuroblastoma and in mice engrafted with human brain lymphoma or multiple myeloma (Rotolo et al., Cancer Cell. 2018; 34 (4): 596-610; Xu et al., Clin Cancer Res. 2019; 25 (23): 7126-7138; Heczey et al., Nat Med. 2020; 26 (11): 1686-1690; Zhu et al., Cell Stem Cell. 2019 (25): 542-557; Li et al., Cell Rep Med. 2021; 2 (11): 100449; Lameris et al., Nat Cancer. 2020; 1 (11): 1054-1065). Advantages of CAR-INKT versus CAR-modified conventional T (CAR-T) cells include 1) that CAR-INKT are capable of infiltering to the tumor more efficiently and inhibiting tumor-associated macrophages (TAMs) and may be more efficacious in solid tumors; 2) that iNKT cells possess metabolic properties that may confer differential functional features and adaptability and longevity to the nutrient-poor tumor microenvironment (TME), and 3) that they do not cause graft versus host disease (GVHD) and can be used as allogenic, off-the-shelf medicine (Song et al., J Clin Invest. 2009; 119 (6): 1524-1536; Khurana et al., Front Immunol., 2021; 12:700374; Nair and Dhodapkar, Front Immunol. 2017; 8:1178; Delfanti et al., Sci Immunol. 2022; 7 (74): eabn6563. doi: 10.1126/sciimmunol.abn6563).

Since human INKT cells in peripheral blood account for about 0.01-1%, it has been a challenge to expand INKT cells for therapeutics. The most common method is to stimulate isolated INKT cells with the irradiated PBMC negative fraction supplemented with αGalCer, IL-2, IL-7, and IL-15, and expanded with αGalCer pulsed autologous dendritic cells (DC) or autologous PBMCs (Poels et al., Int J Mol Sci. 2021; 22:1096; Tian et al., J Clin Invest. 2016; 126 (6): 2341-2355). Isolated INKT cells stimulated with OKT3 anti-CD3 monoclonal antibody, irradiated autologous PBMCs, and IL-2 were used in a clinical trial to treat advanced melanoma (Exley et al., Clin Cancer Res. 2017; 23 (14): 3510-3519). Isolated INKT cells stimulated with αGalCer pulsed autologous PBMCs, IL-2, and IL-21 and modified to express GD2 CAR were used in a clinical trial to treat neuroblastoma (Heczey et al., Nat Med. 2020; 26 (11): 1686-1690). Alternatively, isolated INKT cells were stimulated with autologous PBMCs, anti-CD3/CD28 beads, and IL-15 (Rotolo et al., Cancer Cell. 2018; 34 (10): 596-610).

Adoptive transfer of CAR-T cells against tumor antigens has emerged as a powerful strategy in the treatment of B-cell acute lymphoblastic leukemia (B-ALL), diffuse large B-cell lymphoma (DLBCL), mantal cell lymphoma (MCL) or multiple myeloma (MM) (June and Sadelain., N Engl J Med. 2018: 379 (1): 64-73). However, the major hurdle in developing CAR-T therapy to T-cell malignancies is CAR-T cell fratricide by self-killing due to the shared expression of many targetable antigens (e.g., CD1a, CD2, CD5 and CD7) between CAR-T cells and malignant T cells, leading to insufficient numbers of CAR-T cells for infusion (Scherer et al., Front Oncol. 2019; 9:126; Bayon-Calderon et al., Int J Mol Sci. 2020; 21 (20): 7685).

CRISPR (Clustered Regularly Interspaced Short Palindromic Repeats)-mediated genomic disruption of CD7 and/or TCR alpha chain (TRAC) expression provides an alternative approach to enabling expansion of CD7 CAR transduced T cells from allogeneic healthy donors without extensive self-antigen-driven fratricide. Edited CD7 CAR-T cells produced robust cytotoxicity against malignant T-cell lines and primary tumors and were protective in a mouse xenograft model of acute T-cell leukemia (T-ALL) (Gomes-Silva et al., Blood. 2017; 130 (3): 285-296; Cooper et al., Leukemia. 2018; 32 (9): 1970-1983). However, the safety and clinical implications of CRISPR-based gene editing remains lingering. Studies have reported that CRISPR-Cas9 induce large-scale DNA deletions and chromosomal rearrangements, activation of the p53 tumor suppressor protein and selection for p53 mutations, chromothripies (i.e., chromosome shattering), and large structural variants at on-target and off-target sites in vivo, which are passed on to the next generation (Sheridan C. Nat Biotechnol. 2021; 39 (8): 897-899; 2022; 40 (1): 5-8; Urnov FD. Nat Genet. 2021; 53 (6): 768-769); Hoijer et al., Nat Commun. 2022; 13 (1): 627). Moreover, a recent study has documented high frequencies of chromosome loss and truncation in genetically engineered T cells after CRISPR-Cas9 transfection (Nahmad et al., Nat Biotechnol. 2022; 40 (12): 1807-1813).

T-cell acute lymphoblastic leukemia (T-ALL) is an aggressive hematological disorder. T-ALL subtypes include ETP (early thymic precursors), Pro-T, Pre-T, cortical and mature T-ALL (Bayon-Calderon et al., Int J Mol Sci. 2020; 21 (20): 7685). T-ALL represents approximately 25 and 15% of all newly diagnosed ALL cases in pediatric and adult patients, respectively. Intensive chemotherapy as the standard front-line therapy for T-ALL has raised cure rates to above 85%. However, adult T-ALL still presents a dismal outcome, with significantly lower survival rates than pediatric T-ALL. Children and adolescents with ETP ALL have the poorest response to initial therapy (Cordo et al., Blood Cancer Discov. 2020; 24 (2);19-31; Fleischer et al., J Hematol Oncol. 2019; 12 (1);141; Raetz and Teachey, Hematology Am Soc Hematol Educ Program. 2016; 2016 (1): 580-588). Therefore, new therapies are urgently needed for refractory or relapsed and high-risk patients with T-ALL.

### SUMMARY OF THE DISCLOSURE

The present disclosure relates to invariant natural killer T (INKT) or type I NKT cells (e.g., CD3⁺iTCR(Vα24-Jα18)⁺ or CD3⁺Vα24⁺) that are modified to express a CD7 chimeric antigen receptor (CAR) to treat a CD7⁺ cancer without the use of genome editing (e.g., CRISPR/Cas). In some embodiments, the CD7 CAR-modified INKT cells disclosed herein are CD3⁺iTCR(Vα24-Jα18)⁺ INKT cells (e.g., CD3⁺iTCR(Vα24-Jα18)⁺CD4⁺ cells, CD3⁺iTCR(Vα24-Jα18)⁺CD8⁺ cells, CD3⁺iTCR(Vα24-Jα18)⁺CD4⁻CD8⁻ cells, CD3⁺iTCR(Vα24-Jα18)⁺CD4⁺CD8⁺ cells or a mixture thereof).

The present disclosure further relates to methods and compositions (e.g., pharmaceutical compositions) using the CD7 CAR-modified INKT cells disclosed herein. In some embodiments, the CD7 CAR-modified INKT cells disclosed herein may be useful as therapeutic agents, e.g., in treating or preventing a CD7⁺ cancer (e.g., T-cell leukemias and lymphomas or acute myeloid leukemias). In some embodiments, the INKT cells disclosed herein may be isolated (e.g., from a biological sample, e.g., from a patient or a donor), cultured, modified to express CD7 CAR, and expanded into a cell population. In some embodiments, the CD7 CAR-modified iNKT cells disclosed herein are present and/or used in a pharmaceutical composition.

Pharmaceutical compositions comprising the INKT cells disclosed herein are also provided. In one aspect, the disclosure relates to a pharmaceutical composition comprising isolated CD3⁺iTCR(Vα24-Jα18)⁺ INKT cells according to any embodiment disclosed here and a pharmaceutically acceptable carrier. In some embodiments, the cells are a mixture of CD3⁺iTCR(Vα24-Jα18)⁺CD4⁺ cells, CD3⁺iTCR(Vα24-Jα18)⁺CD8⁺ cells, CD3⁺iTCR(Vα24-Jα18)⁺CD4⁻CD8⁻ cells, and CD3⁺iTCR(Vα24-Jα18)⁺CD4⁺CD8⁺, each optionally isolated from a biological sample of the subject or a donor.

In one embodiment, the disclosure relates to a method of treating or preventing a CD7⁺ cancer in a subject in need thereof, comprising administering to the subject a therapeutically effective amount of CD7 CAR-modified INKT cells according to any embodiment disclosed here, or a pharmaceutical composition comprising a therapeutically effective amount of CD7 CAR-modified INKT cells and a pharmaceutically acceptable carrier.

In another embodiment, the disclosure relates to a method of preparing a therapy for treating or preventing a CD7⁺ cancer in a subject in need thereof, comprising:
a) isolating one or more CD3⁺iTCR(Vα24-Jα18)⁺ INKT cells from a biological sample; and
b) culturing the one or more CD3⁺iTCR(Vα24-Jα18)⁺ INKT cells in a growth medium to express a CD7 CAR and produce an expanded cell population.

In some embodiments, the method further includes modifying the one or more CD3⁺iTCR(Vα24-Jα18)⁺ INKT cells to express a CD7 CAR. In some embodiments, the modifying comprises introducing one or more polynucleotides encoding the CD7 CAR into the one or more cells.

In some embodiments, the CD7 CAR-modified INKT cells are further modified to comprise a CAR, a T-cell receptor (TCR), a TCR mimic antibody (TCRm), an exogenous cytokine, growth factor, antibody or antigen binding fragment, or any combination thereof, wherein the antibody or antigen binding fragment optionally comprises a bispecific T cell engager (BiTE).

In any of the aspects, the cancer that the isolated iNKT cells and modified to express a CD7 CAR can be used to treat or prevent includes, but not limited to, a CD7⁺ cancer or CD7⁺ malignancies; e.g., T-cell lymphoblastic leukemias (T-ALL) and T-ALL subtypes including early thymic precursors (ETP)-ALL (ETP-ALL), Pro-T-ALL, Pre-T-ALL, Cortical T-ALL and Mature T-ALL; a subset of peripheral T-cell lymphomas (PTCLs) and PTCL subtypes including PTCL, not otherwise specified (PTCL-NOS), anaplastic large cell lymphoma (ALCL), primary cutaneous ALCL, angioimmunoblastic T-cell lymphoma (AITL), nasal NK/T-cell lymphoma, adult T-cell acute lymphoblastic lymphoma or leukemia (ATLL) associated with the human T-cell leukemia virus-1 (HTLV-1) infection, enteropathy-associated lymphoma, hepatosplenic lymphoma, subcutaneous panniculitis-like lymphoma, precursor T-cell acute lymphoblastic lymphoma or leukemia, blastic NK-cell lymphoma, and cutaneous T-cell lymphomas (CTCLs); CD7⁺ acute myeloid leukemia (AML); CD7⁺ other malignancies or CD7⁺CD1d⁺ malignancies (e.g., T-ALL).

Other aspects or advantages of the present disclosure can be better understood through the following description of drawings, detailed description of disclosure, examples, and claims.

### BRIEF DESCRIPTION OF THE DRAWINGS

FIG. 1 is a flow cytometric plot showing CD1d expression and NGFR expression in luciferase-expressing tumor cell lines that were used in this disclosure. Tumor cells were transduced with a bidirectional lentiviral vector expressing humanized firefly luciferase and cytoplasmic domain-deleted nerve growth factor receptor (NGFR) (hfflucN). K562, chronic myelogenous leukemia (CML); K562CD19, CD19 stably transfected K562; Daudi and Raji, B-cell lymphoma; Nalm6, B-cell precursor leukemia; HL60, KG1, Mo7e, Molm13, MV4:11, THP1, and U937, acute myeloid leukemia (AML). The data was in part used in PCT/US20/47253.

FIG. 2A is a graph showing the luciferase-based cytotoxicity of INKT1 (auto) and INKT1 (allo) cells at different effector/target (E/T) ratios against luciferase-expressing CD1d⁻ (Daudi, Nalm6) and CD1d⁺ (Molm13, THP1, U937) target cells pre-incubated overnight with DMSO or αGalCer. iNKT1 (auto) and iNKT1 (allo) were generated in the secondary stimulation or expansion by co-culture of iNKT1 cells with irradiated autologous peripheral blood mononuclear cell (PBMC) negative fraction, αGalCer, IL-2, and IL-15 in a 24-well plate or irradiated allogenic PBMCs and Epstein Barr virus transformed B (EBV-B) cells, αGalCer, IL-2, IL-7, and IL-15 in a T25 flask. In the primary stimulation, isolated INKT1 cells from blood donor 1 were co-cultured with irradiated autologous PBMC negative fraction, αGalCer, IL-2, and IL-15 in a 48-well plate.

FIG. 2B is a graph showing the cytotoxicity of iNKT2 (auto) and iNKT2 (allo) cells at different E/T ratios against luciferase-expressing CD1d (Daudi, Nalm6) and CD1d⁺ (KG1, Molm13, MV4:11, THP1) target cells pre-incubated with DMSO or GalCer. INKT2 (auto) and iNKT2 (allo) were generated in the secondary stimulation or expansion by co-culture of INKT2 cells with irradiated autologous PBMC negative fraction, αGalCer, IL-2, and IL-15 in a 24-well plate or irradiated allogenic PBMCs and EBV-B cells, αGalCer, IL-2, IL-7, and IL-15 in a T25 flask. In the primary stimulation, isolated INKT2 cells from blood donor 2 were co-cultured with irradiated autologous PBMC negative fraction, αGalCer, IL-2, and IL-15 in a 48-well plate.

FIG. 3A is a graph showing the specific lysis of CD1d (K562) and CD1d⁺ (U937) target cells pre-incubated with DMSO or αGalCer by iNKT1 cells that were expanded with αGalCer or anti-CD3 antibody (OKT3) plus irradiated allogeneic PBMCs and EBV-B cells, IL-2, IL-7, and IL-15 in a T25 flask.

FIG. 3B is a graph showing the specific lysis of CD1d (K562, Daudi) and CD1d⁺ (HL60, KG1, Molm13, MV4:11, U937) target cells pre-incubated with DMSO or αGalCer by INKT2 cells that were expanded with αGalCer or anti-CD3 antibody (OKT3) plus irradiated allogeneic PBMCs and EBV-B cells, IL2, IL7, and IL-15 in a T25 flask.

FIG. 3C is a graph showing the specific lysis of CD1d (Daudi, Raji) and CD1d⁺ (MV4:11, U937) target cells pre-incubated with DMSO or αGalCer by iNKT12 (αGalCer) and INKT12 (OKT3) cells that were expanded with αGalCer or anti-CD3 antibody (OKT3) plus irradiated allogeneic PBMCs and EBV-B cells, IL-2, IL-7, and IL-15 in a T25 flask. In the primary stimulation, isolated INKT12 cells from blood donor 12 were co-cultured with irradiated autologous PBMC negative fraction, αGalCer, IL-2, and IL-15 in a 48-well plate. INKT2 cells and iNKT1 expanded with αGalCer, irradiated allogeneic PBMCs and EBV-B cells, IL-2, IL-7, and IL-15 in a T25 flask were used as control. E/T ratio of 15:1 in iNKT1 was used.

FIG. 4A is a graph showing the specific lysis of CD1d⁺ (THP1, U937) target cells pre-incubated with DMSO or αGalCer at different E/T ratios by iNKT1, INKT2, INKT11, and INKT12 that were expanded with αGalCer, irradiated allogeneic PBMCs and EBV-B cells, IL2, IL-7, and IL-15 in a T25 flask.

FIG. 4B is a flow cytometric analysis plot showing the percentages of CD3⁺iTCR⁺ cells in expanded iNKT1 and iNKT2 cells (αGalCer or OKT3). ITCR, Vα24-Jα18. Isotype antibodies were used as a background control to set up a gate.

FIG. 5A is a graph showing the cytotoxicity of INKT45 (auto), INKT45 (allo), INKT46 (auto), and iNKT46 (allo) cells against luciferase-expressing CD1d (Nalm6) and CD1d⁺ (MV4:11, U937) target cells pre-incubated with DMSO or αGalCer. INKT45 (auto) or INKT46 (auto) and iNKT45 (allo) or iNKT46 (allo) refer to the expansion of INKT cells that were carried out in a 24-well plate using irradiated autologous PBMC negative fraction or irradiated allogeneic PBMCs and EBV-B cells, respectively, all supplemented with αGalCer, IL-2, and IL-15. INKT cells were then transferred to a T75 flask for further expansion. Isolated iNKT45 and iNKT46 cells from blood donors 45 and 46 were initially stimulated in a 48-well plate with irradiated autologous PBMC negative fraction, αGalCer, IL-2, and IL-15.

FIG. 5B is a flow cytometric analysis plot showing the percentages of CD3⁺iTCR⁺ cells in iNKT45 and iNKT46 cells after primary stimulation. iTCR, Vα24-Jα18. Isotype antibodies were used as a background control to set up a gate.

FIG. 5C is a flow cytometric analysis plot showing the percentages of CD3⁺iTCR⁺ cells in iNKT45 (auto), INKT45 (allo), INKT46 (auto), and iNKT46 (allo) cells after secondary stimulation (i.e., expansion). iTCR, Vα24-Jα18. Isotype antibodies were used as a background control to set up a gate.

FIG. 6 is a graph showing the cytotoxicity of CD19 CAR-modified INKT47 and iNKT48 cells against luciferase-expressing CD19 (K562) and CD19⁺ (Daudi, Nalm6, Raji) as well as DMSO or αGalCer treated CD1d (Mo7e) and CD1d⁺ (Molm13, U937) target cells. Isolated iNKT47 and iNKT48 were modified with CD19 CAR or CD19 CAR/GFP and expanded with irradiated autologous PBMC negative fraction and whole PBMCs or irradiated allogeneic PBMCs and EBV-B cells, all supplemented with αGalCer, IL2, and IL-15.

FIG. 7 is a graph showing production of IFN-γ by CD19 CAR-modified INKT47 and iNKT48 that were expanded with irradiated autologous PBMC negative fraction and whole PBMCs or irradiated allogeneic PBMCs and EBV-B cells in the presence of αGalCer, IL-2, and IL-15 following recognition of CD19⁺ B-cell malignancies (Daudi, Nalm6, Raji) and DMSO or αGalCer treated CD1d (Mo7e) and CD1d⁺ (THP1) target cells in an enzyme-linked immunosorbent assay (ELISA).

FIG. 8 is a flow cytometric analysis plot showing the percentages of CD3⁺iTCR⁺ cells in CD19 CAR-modified INKT47 and iNKT48 cells that were expanded with irradiated autologous PBMC negative fraction and whole PBMCs or irradiated allogeneic PBMCs and EBV-B cells in the presence of αGalCer, IL-2, and IL-15. iTCR, Vα24-Jα18. Isotype antibodies were used as a background control to set up a gate.

FIG. 9 is a flow cytometric analysis plot showing the percentages of CAR⁺ or CAR⁺ GFP⁺ cells in CD19 CAR- or CD19 CAR/GFP-modified INKT47 and iNK&48 cells that were expanded with irradiated autologous PBMC negative fraction and whole PBMCs or irradiated allogeneic PBMCs and EBV-B cells in the presence of αGalCer, IL-2, and IL-15.

FIG. 10 is a graph showing the cytotoxicity of CD19 CAR- or CD19 CAR/GFP-modified INKT50 cells against luciferase-expressing CD19 (K562, HL60, KG1, Molm13, THP1) and CD19⁺ (K562CD19, Daudi, Nalm6, Raji) as well as DMSO or αGalCer treated CD1d (Mo7e) and CD1d⁺ (MV4:11, U937) target cells. Isolated INKT50 cells were modified with lentivirus CD19 CAR or CD19 CAR/GFP and expanded with irradiated autologous PBMC negative fraction and whole PBMCs or irradiated allogeneic PBMCs and EBV-B cells in the presence of αGalCer, IL-2, and IL-15.

FIG. 11A is a flow cytometric analysis plot showing the percentages of CD3⁺iTCR⁺ cells in CD19 CAR-modified INKT50 or mock cells that were expanded with irradiated autologous PBMC negative fraction and whole PBMCs or irradiated allogeneic PBMCs and EBV-B cells in the presence of αGalCer, IL2, and IL-15. iTCR, Vα24-Jα18. Isotype antibodies were used as a background control to set up a gate.

FIG. 11B is a flow cytometric analysis plot showing the percentages of CAR⁺ or CAR⁺ GFP⁺ cells in CD19 CAR- or CD19 CAR/GFP-modified INKT50 or mock cells that were expanded with irradiated autologous PBMC negative fraction and whole PBMCs or irradiated allogeneic PBMCs and EBV-B cells in the presence of αGalCer, IL-2, and IL-15.

FIG. 12 is a graph showing the expansion rate of CD19 CAR- or CD19 CAR/GFP-modified INKT47, INKT48, and iNKT50 or unmodified iNKT45 and iNKT46 cells that were expanded with irradiated autologous PBMC negative fraction and whole PBMCs or irradiated allogeneic PBMCs and EBV-B cells in the presence of αGalCer, IL-2, and IL-15.

FIG. 13A is a graph showing the overnight (16 hour) cytotoxicity of thawed CD19 CAR-modified iNKT47 and iNKT48 that were expanded with irradiated allogeneic PBMCs and EBV-B cells in the presence of αGalCer, IL2, and IL15 against CD19⁻ (K562) and CD19⁺ (K562CD19, Daudi, Nalm6, Raji) target cells. CD19 CAR-modified INKT47 (allo) and iNKT48 (allo) were thawed and cultured in a 24-well plate in human T-cell medium supplemented with IL-2 and IL-15 for 5 days.

FIG. 13B is a graph showing the overnight (16 hour) cytotoxicity of thawed CD19 CAR-modified INKT47 cells that were expanded with irradiated allogeneic PBMCs and EBV-B cells in the presence of αGalCer, IL2, and IL-15 against luciferase-expressing Molm13 and THP1 cells in the presence of 7DW8-5 or αGalCer at varied concentrations ranging from 200 ng/ml to 3.12 ng/ml. CD19 CAR-modified INKT47 (allo) cells were thawed and cultured in a 24-well plate in human T-cell medium supplemented with IL-2 and IL-15 for 14 days. E/T ratio, 10/1.

FIG. 13C is graph showing IFN-γ production of INKT47 CD19 CAR (allo), INKT48 CD19 CAR/GFP (allo) cells, and iNKT47 mock (auto) cultured at day 92 in response to CD1d⁺ MV4:11-hfflucN target cells pulsed in different amounts of 7DW8-5 and αGalCer. CD19⁺ (K562CD19, Nalm6) and CD19⁻ (K562) were used as positive and negative controls.

FIG. 14A is a flow cytometric analysis plot showing the percentages of CD2 versus CD7 and CD5 versus CD7 positive cell populations in expanded INKT cells with irradiated allogeneic PBMCs and EBV-B cells, αGalCer or 7DW8-5, IL-2, and IL-15 from one representative healthy blood donor (INKT69).

FIG. 14B is a graph showing statistical analysis of flow cytometric data of CD2, CD5 and CD7 positive cell populations in expanded INKT cell products from 7-9 individual blood donors (INKT45, 46, 47, 48, 50, 55, 56, 68, 69 except for iNKT47, 48 in the case of CD2) that were expanded with irradiated allogeneic PBMCs and EBV-B cells, αGalCer or 7DW8-5, IL-2, and IL-15. The results were calculated as mean±SD. **** p<0.0001.

FIG. 15 is a flow cytometric analysis plot showing CD7, CD1d and NGFR expression in bidirectional (humanized firefly luciferase and NGFR) lentiviral transduced human HSB2, Jurkat, and MOLT13 T-ALL cell lines as well as KG1, Kasumi-1, and Kasumi-6 AML cell lines.

FIG. 16 is a graph showing T cell expansion rate after lentiviral transduction of peripheral blood mononuclear cells (PBMCs) from a donor 36 (PBL36) with CD19, ROR1 and CD7 CAR. Untransduced mock was used as control.

FIG. 17 is a graph showing iNKT cell expansion rate after lentiviral transduction of INKT55 cells isolated from a donor 55 with CD7 CAR, CD19 CAR or untransduced mock control and iNKT68 isolated from a donor 68 with CD7 CAR or untransduced mock control. INKT55 and iNKT68 mock or CD19 CAR or CD7 CAR transduced were expanded with irradiated allogeneic PBMCs and EBV-B cells, 7DW8-5, IL-2, and IL-15.

FIG. 18 is a graph showing the representative cytotoxicity of CD7 CAR INKT55 cells derived from a donor 55 (mock iNKT55, CD19 CAR INKT55 and CD7 CAR INKT55 cells) on luciferase-expressing target cells including K562 (CML), CD19⁺ B-cell malignancies (Nalm6, Raji), CD7⁺ T-ALL (HSB2, Jurkat, MOLT13), CD7⁺ AML (KG1), CD7⁻ AML (Molm13, MV4; 11, U937), CD7⁻ soft tissue sarcoma (Rh30, TC71, SaOS2) and CD7⁻ neuroblastoma (BE (2) C, SKNFI) cell lines. E:T (Effector:Target) ratio.

FIG. 19 is a graph showing statistical analysis of five independent cytotoxicity assays of CD7 CAR INKT cells (n=5) derived from donors 55, 56, 64, 68, and 69 on luciferase-expressing CD7⁺ T-ALL cell lines (HSB2, Jurkat, MOLT13), CD7⁺ AML (KG1) and CD19⁺ B-cell malignancies (Nalm6, Raji). Corresponding mock (n=5) and CD19 CAR (n=3) INKT cells were used as baseline and CAR-T cell controls. The results were calculated as mean±SD. * p<0.05, ** p<0.01, *** p<0.001, **** p<0.0001, ns, not significant. Significances between CD19 CAR INKT vs. mock and CD7 CAR INKT vs. mock are indicated.

FIG. 20A is a graph showing production of IFN-γ by representative CD7 CAR INKT55 and mock iNKT55 or CD19 CAR INKT55 control following recognition of a panel of CD7⁺ T-ALL (HSB2, Jurkat, MOLT13), CD7⁺ AML (KG1, Kasumi-6), CD19⁺ B-cell malignancies (Daudi, Nalm6, Raji) and CD19⁺ EBV-transformed B-cells (EBV-B) used for CD19 CAR INKT55 control, and CD7⁻ CML (K562), AML (Kasumi-1, U937), multiple myeloma (RPMI8226), sarcoma (Rh30, TC71), and neuroblastoma (BE2 (C), SKNFI) cell lines in an enzyme-linked immunosorbent assay (ELISA).

FIG. 20B is a graph showing statistical analysis of five independent IFN-γ production assays of CD7 CAR INKT cells (n=5) manufactured from donors 55, 56, 64, 68, and 69 in response to CD7⁺ T-ALL (HSB2, Jurkat, MOLT13) and CD7⁺ AML (KG1, Kasumi-6), CD19⁺ B-cell malignancies (Daudi, Nalm6, Raji), and CD7⁻ CML (K562) and AML (Kasumi-1, U937). Corresponding mock (n=5) and CD19 CAR (n=3) iNKT cells were used as baseline and CAR-T cell controls. The results were calculated as mean±SD. * p<0.05, ** p<0.01, *** p<0.001, **** p<0.0001, ns, not significant. Statistical significances between mock and CD7 CAR INKT were indicated.

FIG. 21 is a flow cytometric analysis plot showing the level of CD7 CAR expression after lentiviral transduction in INKT cells derived from five blood donors (n=5). CD19 CAR INKT (n=3) and mock iNKT (n=5) cells were used as specificity or background controls.

FIG. 22A is a flow cytometric dot plot of CD2 versus CD7 and CD5 versus CD7 double staining showing an exemplary elimination of CD7⁺ cells in final CD7 CAR INKT55 products. Mock and CD19 CAR INKT55 were used as controls.

FIG. 22B is a graph showing statistical analysis of the percentages of remaining CD7⁺ cell populations in final CD7 CAR INKT cell products (n=4) manufactured from donors 55, 56, 68, and 69 compared to mock (n=4) and irrelevant CD19 CAR (INKT 55, INKT56) or ROR1 CAR (iNKT69) INKT cells (n=3). The results were calculated as mean±SD. ** p<0.01, ns, not significant.

FIG. 23A is a flow cytometric analysis plot showing the percentages of CD3⁺iTCR(Vα24-Jα18)⁺ cell populations in CD7 CAR INKT, mock INKT and CD19 CAR INKT cells from one representative donor 55. Isotype control antibodies were used to set up a background gate.

FIG. 23B is a flow cytometric analysis plot showing the percentages of CD4⁺, CD8⁺, CD4⁻CD8⁻, and CD4⁺CD8⁺ cell populations in CD7 CAR INKT, mock and CD19 CAR INKT cells from one representative donor 55. Isotype control antibodies were used to set up a background gate.

FIG. 23C is a flow cytometric histogram analysis showing the level of PD1 expression in CD7 CAR INKT, mock and CD19 CAR INKT cells from one representative donor 55.

FIG. 24A is a graph showing the experimental schedule of HSB2-hfflucN tumor cell injection, INKT cell infusion and bioluminescent imaging (BLI) monitoring. HSB2-hfflucN T-ALL cells were i.v. injected into 6-7-week-old female NSG mice at day-2. Tumor growth was determined by BLI at day 0. The mice were randomly divided into three groups (n=5 each) and treated with three i.v. infusions of PBS, thawed mock iNKT55 or thawed CD7 CAR INKT55 cells at day 0, 3 and 6. PBS or IL-2 and IL-15 were i.p. injected into mice every three days for two weeks. Tumor growth was monitored by BLI at day 3, 6, 10, 17 and 24.

FIG. 24B is a graph showing bioluminescent imaging (BLI) of tumor growth in mice (three groups, n=5 each) treated with PBS, mock iNKT55 cells or CD7 CAR INKT55 cells. The scale bar is indicated as radiance (p/sec/cm²/sr). All PBS or mock control mice died at day 23. One mouse (the first one) in CD7 CAR INKT treated group at day 24 died right after imaging likely due to the anesthetic procedure for imaging or injection of luciferin.

FIG. 24C is a graph showing overall kinetics of systemic tumor progression in mice. Each line donates an individual animal.

FIG. 24D is a graph showing statistical analysis of tumor progression as determined by BLI imaging in each group of mice. P-values <0.05 considered significant, *p<0.05, ** p<0.01, *** p<0.001, **** p<0.0001, ns, not significant.

FIG. 24E is a graph showing a Kaplan-Meier survival curve of mice treated with PBS, mock iNKT or CD7 CAR INKT cells. P=0.0009, by log-rank Mantel-Cox test.

FIG. 25A is a graph showing the experimental schedule of KG1-hfflucN tumor cell injection, INKT cell infusion and bioluminescent imaging (BLI) monitoring. KG1-hfflucN T-ALL cells were i.v. injected into 6-7-week-old female NSG mice at day-2. Tumor growth was determined by BLI at day 0. The mice were randomly divided into two groups (n=5 each) and treated with two i.v. infusions of thawed mock INKT55 or thawed CD7 CAR INKT55 cells at day 0 and 3. PBS or IL-2 and IL-15 were i.p. injected into mice every three days for two weeks. Tumor growth was monitored by BLI on day 3, 6, 10, 17, 24, 31, 38 and 45.

FIG. 25B is a graph showing bioluminescent imaging (BLI) of tumor growth in mice (two groups, n=5 each) treated with mock iNKT55 cells or CD7 CAR INKT55 cells. The scale bar is indicated as radiance (p/sec/cm²/sr). The third mouse in CD7 CAR INKT treated group showed larger tumor mass than other mice in the same group. This might be caused by i.p. injection of remaining T cells in the syringe during the 2nd i.v. T-cell infusion. One mouse (the first one) in CD7 CAR INKT treated group at day 31 died right after imaging likely due to the anesthetic procedure for imaging or injection of luciferin.

FIG. 25C is a graph showing overall kinetics of systemic tumor progression in mice. Each line donates an individual animal.

FIG. 25D is a graph showing statistical analysis of tumor progression as determined by BLI imaging in each group of mice. P-values <0.05 considered significant, *p<0.05, ** p<0.01, *** p<0.001, **** p<0.0001, ns, not significant.

FIG. 25E is a graph showing a Kaplan-Meier survival curve of mice treated with mock INKT or CD7 CAR INKT cells. P=0.0069, by log-rank Mantel-Cox test. One mouse (the first one) in CD7 CAR INKT treated group died at day 31 after imaging was not counted in the survival curve. All control mice died on day 73-74. Two mice in CD7 CAR INKT treated group survived for 368 days and were humanely euthanized.

### DETAILED DESCRIPTION OF THE DISCLOSURE

The present disclosure is based on the recognition that type I NKT cells or invariant NKT (INKT) cells (e.g., CD3⁺iTCR(Vα24-Jα18)⁺ or CD3⁺Vα24⁺) express about 42.96%±17.21% CD7⁻ subsets in INKT cells and can be expanded in culture for approximately 6,000-fold after lentiviral modification to express CD7 chimeric antigen receptor (CAR). Thus, CD7 CAR-modified CD7⁻ INKT cells can avoid CD7 antigen-driven fratricide or self-killing and be expanded for therapeutics. Importantly, CD7 CAR-modified INKT cells exhibit anti-CD7⁺ cancer activity in vitro and in animals. Moreover, about 6,000-fold expansion of INKT cells or INKT cells modified with CD19 CAR can also be achieved using irradiated allogeneic PBMCs and EBV-B cells, αGalCer or 7DW8-5, IL-2, and IL-15. The present disclosure provides several advantages. First, CD7 CAR-modified INKT cells are generated without the use of genome editing and while most CD7 CAR-modified conventional T cells require CRISPR-mediated genome editing. Therefore, CD7 CAR-modified INKT cells might be safer than CD7 CAR modified conventional T cells by avoiding genome editing-associated off-target effect. Second, CD7 CAR-modified INKT cells may survive in vivo for weeks to months while most CD7 CAR-modified conventional T cells may persist for months to years and could likely result in more severe T cell deficiency and infection. Third, CD7 CAR-modified INKT cells may provide dual targeting for CD7⁺CD1d⁺ T-ALL by CD7 CAR and invariant TCR and are probably more effective in terms of lowering a relapse rate in those patients than CD7 CAR-modified conventional T cells. Fourth, CD7 CAR modified iNKT cells can be manufactured from healthy donors as off-the-shelf without causing graft versus host disease (GVHD) and the need of genome editing while CRISPR-mediated deletion of both CD7 and allo-reactive TCR is required for CD7 CAR-modified conventional T cells.

Accordingly, in certain aspects, the present disclosure provides methods and compositions (e.g., pharmaceutical compositions) using CD7 CAR-modified INKT cells for treating or preventing a CD7⁺ cancer in a subject in need thereof. In some embodiments, the CD7 CAR-modified INKT cells comprise or consist of CD3⁺iTCR(Vα24-Jα18)⁺ INKT cells. In some embodiments, the CD7 CAR-modified INKT cells comprise or consist of CD3⁺Vα24⁺ cells. In some embodiments, the CD7 CAR-modified INKT cells comprise or consist of CD3⁺CD4⁺ cells. In some embodiments, the CD7 CAR-modified INKT cells comprise or consist of CD3⁺CD4⁻CD8⁻ cells. In some embodiments, the CD7 CAR-modified INKT cells comprise or consist of CD3⁺CD8⁺ cells. In some embodiments, the CD7 CAR-modified INKT cells comprise or consist of a mixture of CD3⁺CD4⁺ cells, CD3⁺CD4⁻CD8⁻, CD3⁺CD8⁺ cells, and CD3⁺CD4⁺CD8⁺ cells. In some embodiments, the CD7 CAR-modified INKT cells are reactive to α-galactosylceramide (αGalCer) or 7DW8-5 or other glycolipid analog presented by MHC class I-like CD1d molecules.

In some embodiments, the exemplary CD7 CAR-modified INKT cells described herein can be used to treat and/or prevent a CD7⁺ cancer (e.g., T-cell acute lymphoblastic leukemia (T-ALL), acute myeloid leukemias (AML), e.g., refractory or relapsed T-ALL or AML). In some embodiments, the CD7⁺ cancer is T-ALL or AML. In some embodiments, the CD7⁺ cancer is refractory or relapsed T-ALL or AML. In some embodiments, the CD7⁺ cancer is relapsed T-ALL or AML, e.g., after hematopoietic stem cell transplantation. In some embodiments, the CD7⁺ cancer is a cancer that expresses both CD7 and an additional antigen targeted by the INKT cells and/or by a construct expressed by the iNKT cells (e.g., a chimeric antigen receptor (CAR), a T cell receptor (TCR), or a T cell receptor mimic antibody (TCRm), or a combination thereof). In some embodiments, the CD7⁺ cancer is a cancer that is resistant or refractory to treatment in the absence of the cells. Such exemplary cancers are described and exemplified herein.

In some embodiments, the cancer is a CD7⁺ hematological malignancy; T-cell lymphoblastic leukemias (T-ALL) and T-ALL subtypes including early thymic precursors (ETP)-ALL (ETP-ALL), Pro-T-ALL, Pre-T-ALL, Cortical T-ALL and Mature T-ALL; a subset of peripheral T-cell lymphomas (PTCLs) and PTCL subtypes including PTCL, not otherwise specified (PTCL-NOS), anaplastic large cell lymphoma (ALCL), primary cutaneous ALCL, angioimmunoblastic T-cell lymphoma (AITL), nasal NK/T-cell lymphoma, adult T-cell acute lymphoblastic lymphoma or leukemia (ATLL) associated with the human T-cell leukemia virus-1 (HTLV-1) infection, enteropathy-associated lymphoma, hepatosplenic lymphoma, subcutaneous panniculitis-like lymphoma, precursor T-cell acute lymphoblastic lymphoma or leukemia, blastic NK-cell lymphoma, and cutaneous T-cell lymphomas (CTCLs); CD7⁺ acute myeloid leukemia (AML); CD7⁺ other malignancies or CD7⁺CD1d⁺ malignancies, and wherein optionally the CD7⁺ cancer is resistant or refractory to treatment in the absence of the cells.

In some embodiments, the present disclosure provides a method of treating or preventing a CD7⁺ cancer in a subject in need thereof, comprising administering to the subject a therapeutically effective amount of CD7 CAR-modified INKT cells (e.g., CD3⁺iTCR(Vα24-Jα18)⁺ or CD3⁺Vα24⁺ INKT cells), or a pharmaceutical composition comprising a therapeutically effective amount of CD7 CAR-modified INKT cells (e.g., CD3⁺iTCR(Vα24-Jα18)⁺ or CD3⁺Vα24⁺ INKT cells). In some embodiments, the pharmaceutical composition further comprises at least one pharmaceutically acceptable carrier.

In some embodiments, the present disclosure provides a method of preparing a therapy for treating or preventing a CD7⁺ cancer in a subject in need thereof, comprising: (a) isolating one or more iNKT cells (e.g., CD3⁺iTCR(Vα24-Jα18)⁺ or CD3⁺Vα24⁺ INKT cells) from a biological sample; (b) enabling or activating the one or more iTCR(Vα24-Jα18)⁺ INKT cells in a growth medium for proliferation using irradiated autologous PBMC negative fraction and/or whole PBMCs, α-galactosylceramide (αGalCer) or 7DW8-5 or other glycolipid analog, IL-2, and IL-15 or IL-2, IL-7, and IL-15; (c) modifying activating or proliferating iNKT cells to express CAR (e.g., CD7) using a lentiviral vector; and (d) expanding the one or more CAR (e.g., CD7) modified iNK T cells in a growth medium using irradiated allogeneic PBMCs and EBV-B cells, αGalCer or 7DW8-5, IL-2, and IL-15 or IL-2, IL-7, and IL-15.

In some embodiments, the method further comprises modifying the one or more cells to express a CAR, TCR, or TCRm. In some embodiments, the modifying comprises introducing one or more polynucleotides encoding the CAR, TCR, or TCRm into the one or more cells. In some embodiments, introducing one or more polynucleotides comprises electroporation, transduction, and/or transfection. In some embodiments, the one or more polynucleotides comprise mRNA and/or DNA. In some embodiments, the DNA comprises transposon DNA. In some embodiments, the one or more polynucleotides comprise one or more vectors. In some embodiments, the one or more vectors comprise one or more viral vectors. In some embodiments, the one or more vectors comprise one or more lentiviral vectors or γ-retroviral vectors.

In some embodiments, the INKT cells are isolated from a biological sample. In some embodiments, the biological sample is from the subject (e.g., a cancer patient). In some embodiments, the biological sample is from a donor (e.g., a healthy donor). In some embodiments, the biological sample comprises blood, bone marrow, lymph node tissue, spleen tissue, tumor tissue, one or more induced pluripotent stem cells, and/or one or more peripheral blood mononuclear cells. In some embodiments, the blood comprises peripheral blood and/or umbilical cord blood. In some embodiments, the INKT cells are isolated from one or more peripheral blood mononuclear cells.

In some embodiments of the methods described herein, INKT cells are modified to express a chimeric antigen receptor (CAR) (e.g., CD7 CAR). In some embodiments, the cells comprise one or more polynucleotides encoding the CAR. In some embodiments, the CAR comprises at least an antigen binding domain, a hinge domain, a transmembrane domain, and an intracellular signaling domain.

In some embodiments, an antigen binding domain of a CAR is capable of binding to CD7, CD1a, CD1d, CD2, CD5, TRBC1, CD21, CCR9, CD30, CD123, CD33, CD38, CD138, CLL-1, LILRB4, Siglec-6, CD70, or PD-L1. In some embodiments, the antigen binding domain is capable of binding to CD19 or ROR1.

In some embodiments, the antigen binding domain and/or CAR is capable of binding to CD7. In some embodiments, the antigen binding domain and/or CAR is capable of binding to CD1a. In some embodiments, the antigen binding domain and/or CAR is capable of binding to CD1d. In some embodiments, the antigen binding domain and/or CAR is capable of binding to CD2. In some embodiments, the antigen binding domain and/or CAR is capable of binding to CD5. In some embodiments, the antigen binding domain and/or CAR is capable of binding to TRBC1. In some embodiments, the antigen binding domain and/or CAR is capable of binding to CD21. In some embodiments, the antigen binding domain and/or CAR is capable of binding to CCR9. In some embodiments, the antigen binding domain and/or CAR is capable of binding to CD30. In some embodiments, the antigen binding domain and/or CAR is capable of binding to CD123. In some embodiments, the antigen binding domain and/or CAR is capable of binding to CD33. In some embodiments, the antigen binding domain and/or CAR is capable of binding to CD38. In some embodiments, the antigen binding domain and/or CAR is capable of binding to CD138. In some embodiments, the antigen binding domain and/or CAR is capable of binding to CLL-1. In some embodiments, the antigen binding domain and/or CAR is capable of binding to LILRB4. In some embodiments, the antigen binding domain and/or CAR is capable of binding to Siglec-6. In some embodiments, the antigen binding domain and/or CAR is capable of binding to CD70. In some embodiments, the antigen binding domain and/or CAR is capable of binding to PD-L1.

In some embodiments, the antigen binding domain and/or CAR is capable of binding to CD7, CD1a, CD1d, CD2, CD5, TRBC1, TRBC2, CD21, CCR9, CD30, or CD70 and the cancer is T-cell acute lymphoblastic leukemia (T-ALL) and T-cell lymphoma. In some embodiments, the antigen binding domain and/or CAR is capable of binding to CD123, CD33, CD38, CD138, CLL-1, LILRB4, Siglec-6, or CD70 and the cancer is acute myeloid leukemia (AML). In some embodiments, the antigen binding domain and/or CAR is capable of binding to CD70 and the cancer is T-cell leukemia and lymphoma, multiple myeloma, AML, diffuse large B-cell and follicular lymphoma, Hodgkin lymphoma, or a variety of solid tumors (e.g., renal cell carcinoma, nasopharyngeal carcinoma, glioblastoma, melanoma, glioma, lung cancer, breast cancer, cervix carcinoma, ovarian carcinoma, and mesothelioma). In some embodiments, the antigen binding domain and/or CAR is capable of binding to PD-L1 and the cancer is T-cell leukemia and lymphoma, multiple myeloma, AML, or a variety of solid tumors (e.g., glioma, lung cancer, breast cancer).

In some embodiments, the antigen binding domain and/or CAR is capable of binding to CD19 and the cancer is a B-cell malignancy (e.g., B-cell precursor acute lymphoblastic leukemia (B-ALL), non-Hodgkin lymphoma (NHL), or chronic lymphocytic leukemia (CLL)). In some embodiments, the antigen binding domain and/or CAR is capable of binding to CD19 and the cancer is B-ALL, NHL, or CLL. In some embodiments, the antigen binding domain and/or CAR is capable of binding to ROR1 and the cancer is Ewing sarcoma, osteosarcoma, fibrosarcoma, rhabdomyosarcoma, chronic lymphocytic leukemia, mantle cell carcinoma, breast cancer, lung adenocarcinoma, melanoma, neuroblastoma, or ovarian cancer.

In some embodiments, an antigen binding domain of a CAR comprises an antibody or an antigen binding fragment thereof, or a non-antibody protein scaffold. In some embodiments, the antibody is a monoclonal antibody, a polyclonal antibody, a synthetic antibody, a human antibody, a humanized antibody, or a single domain antibody (known as a nanobody). In some embodiments, the antigen binding fragment comprises a single chain variable fragment (scFv).

In some embodiments, an intracellular signaling domain of a CAR comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1BB, CD28, CD27, CD134 (OX40), ICOS, DAP10, or DAP12.

In some embodiments of the methods described herein, CD7 CAR-modified INKT cells are further modified to express a T cell receptor (TCR). In some embodiments, the cells comprise one or more polynucleotides encoding the TCR. In some embodiments, the TCR comprises at least an alpha chain and a beta chain. In some embodiments, the alpha chain and/or the beta chain is capable of binding to an antigen. In some embodiments, the antigen is an intracellular antigen. In some embodiments, the antigen is Wilm's tumor 1 (WT1), preferentially expressed antigen in melanoma (PRAME), minor histocompatibility antigen (MiHA, e.g., HA-1), or mutated nucleophosmin 1 (ΔNPM1).

In some embodiments, an alpha chain and/or a beta chain of a TCR is capable of binding to WT1, PRAME, HA-1, or ΔNPM1. In some embodiments, the alpha chain, beta chain, and/or TCR is capable of binding to WT1. In some embodiments, the alpha chain, beta chain, and/or TCR is capable of binding to PRAME. In some embodiments, the alpha chain, beta chain, and/or TCR is capable of binding to HA-1. In some embodiments, the alpha chain, beta chain, and/or TCR is capable of binding to ΔNPM1. In some embodiments, the antigen is WT1, PRAME, HA-1 or ΔNPM1 and the cancer is AML.

In some embodiments of the methods described herein, CD7 CAR-modified INKT cells are further modified to express a T cell receptor mimic antibody (TCRm). In some embodiments, the cells comprise one or more polynucleotides encoding the TCRm. In some embodiments, the TCRm comprises at least an antigen binding domain, a hinge domain, a transmembrane domain, and an intracellular signaling domain.

In some embodiments, an antigen binding domain of a TCRm is capable of binding to a composite antigen. In some embodiments, a composite antigen comprises a peptide and a human leukocyte antigen (HLA) molecule.

In some embodiments, an HLA molecule is a class I HLA molecule. In some embodiments, an HLA molecule is a class II HLA molecule.

In some embodiments, a peptide comprises a WT1 peptide. In some embodiments, the WT1 peptide comprises an amino acid sequence of RMFPNAPYL. In some embodiments, the composite antigen comprises a WT1 peptide and an HLA-A2 molecule. In some embodiments, the cancer is AML.

In some embodiments, a peptide comprises a preferentially expressed antigen in melanoma (PRAME) peptide. In some embodiments, the PRAME peptide comprises an amino acid sequence of VLDGLDVLL. In some embodiments, the PRAME peptide comprises an amino acid sequence of ALYVDSLFFL. In some embodiments, the PRAME peptide comprises an amino acid sequence of SLYSFPEPEA. In some embodiments, the PRAME peptide comprises an amino acid sequence of SLLQHLIGL. In some embodiments, the PRAME peptide comprises an amino acid sequence of LYVDSLFFLC. In some embodiments, the composite antigen comprises a PRAME peptide and an HLA-A*0201 molecule. In some embodiments, the composite antigen comprises a PRAME peptide and an HLA-A*2402 molecule. In some embodiments, the cancer is AML, multiple myeloma, T cell lymphoma, B-ALL, neuroblastoma, sarcoma, melanoma, non-small cell lung cancer, colon adenocarcinoma, or breast adenocarcinoma.

In some embodiments, a peptide comprises an HA-1 peptide. In some embodiments, the HA-1 peptide comprises an amino acid sequence of VLHDDLLEA. In some embodiments, the composite antigen comprises an HA-1 peptide and an HLA-A2 molecule. In some embodiments, the cancer is AML and multiple myeloma.

In some embodiments, a peptide comprises a ΔNPM1 peptide. In some embodiments, the ΔNPM1 peptide comprises an amino acid sequence of CLAVEEVSL. In some embodiments, the composite antigen comprises a ΔNPM1 peptide and an HLA-A2 molecule. In some embodiments, the cancer is AML.

In some embodiments, an antigen binding domain of a TCRm comprises an antibody or an antigen binding fragment thereof, or a non-antibody protein scaffold. In some embodiments, the antibody is a monoclonal antibody, a polyclonal antibody, a synthetic antibody, a human antibody, a humanized antibody, or a single domain antibody (a nanobody). In some embodiments, the antigen binding fragment comprises a single chain variable fragment (scFv).

In some embodiments, an intracellular signaling domain of a TCRm comprises a functional signaling domain of at least one stimulatory molecule. In some embodiments, the at least one stimulatory molecule comprises a zeta chain associated with a T cell receptor complex. In some embodiments, the at least one stimulatory molecule comprises a CD3 zeta chain. In some embodiments, the intracellular signaling domain further comprises a functional signaling domain of at least one costimulatory molecule. In some embodiments, the at least one costimulatory molecule comprises 4-1BB, CD28, CD27, CD134 (OX40), ICOS, DAP10, or DAP12.

In some embodiments of the methods described herein, CD7 CAR-modified INKT cells are further modified to comprise an exogenous cytokine, growth factor, antibody or antigen binding fragment, or any combination thereof. In some embodiments, the antibody or antigen binding fragment comprises a bispecific T cell engager (BiTE).

Further provided herein, in some embodiments, are pharmaceutical compositions comprising CD7 CAR-modified INKT cells (e.g., CD3⁺iTCR(Vα24-Jα18)⁺ or CD3⁺Vα24⁺ INKT cells). In some embodiments, the present disclosure provides a pharmaceutical composition for treating or preventing a CD7⁺ cancer in a subject in need thereof comprising CD7 CAR-modified INKT cells (e.g., CD3⁺iTCR(Vα24-Jα18)⁺ or CD3⁺Vα24⁺ INKT cells) and at least one pharmaceutically acceptable carrier. In some embodiments, the CD7 CAR-modified INKT cells in the pharmaceutical composition are CD3⁺iTCR(Vα24-Jα18)⁺CD7 CAR⁺ INKT cells (e.g., CD3⁺CD4⁺ cells, CD3⁺CD4⁻CD8⁻, CD3⁺CD8⁺ cells, CD3⁺CD4⁺CD8⁺ or a mixture thereof). In some embodiments, the CD7 CAR-modified INKT cells in the pharmaceutical composition are CD3⁺CD4⁺ cells. In some embodiments, the CD7 CAR-modified INKT cells in the pharmaceutical composition are CD3⁺CD4⁻CD8⁻ cells. In some embodiments, the CD7 CAR-modified INKT cells in the pharmaceutical composition are CD3⁺CD8⁺ cells. In some embodiments, the CD7 CAR-modified INKT cells in the pharmaceutical composition are CD3⁺CD4⁺CD8⁺ cells. In some embodiments, the CD7 CAR-modified INKT cells in the pharmaceutical composition are CD3⁺CD4⁺ cells, CD3⁺CD4⁻ CD8⁻ cells, CD3⁺CD8⁺ and CD3⁺CD4⁺CD8⁺ cells.

Also provided herein, in some embodiments, are therapeutic uses for CD7 CAR-modified INKT cells (e.g., CD3⁺iTCR(Vα24-Jα18)⁺ or CD3⁺Vα24⁺ INKT cells), or pharmaceutical compositions comprising the same.

### CERTAIN ILLUSTRATIVE EMBODIMENTS

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein are also incorporated by reference in their entirety. To the extent a cited reference conflicts with the disclosure herein, the specification shall control.

As used herein, the singular forms of a word also include the plural form, unless the context clearly dictates otherwise; as examples, the terms “a,” “an,” and “the” are understood to be singular or plural. By way of example, “an element” means one or more element. The term “or” shall mean “and/or” unless the specific context indicates otherwise. All ranges include the endpoints and all points in between unless the context indicates otherwise.

The term “about” or “approximately,” as used herein in the context of numerical values and ranges, refers to values or ranges that approximate or are close to the recited values or ranges such that the embodiment may perform as intended, as is apparent to the skilled person from the teachings contained herein. This is due, at least in part, to the varying properties of nucleic acid compositions, age, race, gender, anatomical and physiological variations and the inexactitude of biological systems. Thus, these terms encompass values beyond those resulting from systematic error. In some embodiments, “about” or “approximately” means plus or minus (±) 10%, sometimes preferably ±5%, and sometimes more preferably ±2%, of a numerical amount.

### NKT Cells

In certain aspects, the present disclosure provides CD7 CAR-modified type I NKT cells (e.g., CD3⁺iTCR(Vα24-Jα18)⁺ or CD3⁺Vα24⁺ INKT cells), as well as methods and compositions using the CD7 CAR-modified INKT cells described herein.

As used herein, the term “natural killer T cell” or “NKT cell,” refers to a T cell or a T cell population that exhibits characteristics of both conventional T cells and natural killer (NK) cells. For instance, in some embodiments, an NKT cell is a mature lymphocyte that bears both T and NK cell receptors. In some embodiments, an NKT cell arises in the thymus from CD4⁺CD8⁺ cortical thymocytes that have undergone T cell receptor (TCR) gene rearrangement.

There are two classifications of NKT cells in literatures. One classic classification of NKT cells refers NKT cells to a subgroup of unconventional T cells that recognize lipid antigens presented by MHC class I-like CD1d molecules and divides NKT cells into type I and type II NKT cells (Godfrey et al. Nat Immunol. 2010; 11 (3): 197-206; Dhodapkar and Kumar. J Immunol. 2017; 198 (3): 1015-21; Godfrey et al. Immunity. 2018; 48 (3): 453-73). Another classification of NKT cells includes type I, type II and type III NKT (NKT-like) cells (Godfrey et al. Nat Rev Immunol. 2004; 4 (3): 231-237; Farr et al. Proc Natl Acad Sci USA. 2014; 111 (35): 12841-6).

As used herein, the term “type I NKT cell” or “invariant NKT cell” or “INKT cell” refers to an NKT cell or an NKT cell population that expresses an invariant or semi-invariant TCR repertoire and binds to the glycosphingolipid α-galactosylceramide (α-GalCer) in association with MHC class I-like CD1d molecules. In some embodiments, a type I NKT cell expresses an invariant TCRα-chain and a limited number of non-invariant TCRβ-chains. In some embodiments, a type I NKT cell expresses a semi-invariant Vα chain (e.g., Vα14-Jα18 TCR in mice, and Vα24-Jα18 in humans), paired with a limited repertoire of Vβ-chains (e.g., Vβ8.2, Vβ7, and Vβ2 in mice, and Vβ11 in humans). In some embodiments, a type I NKT cell recognizes the glycosphingolipid α-galactosylceramide (α-GalCer) or a synthetic analog (e.g., 7DW8-5) thereof when presented by MHC class I-like CD1d molecules. In some embodiments, the term “type I NKT cells” or “invariant NKT cell” or “INKT cell” may be used interchangeably.

As used herein, the term “iTCR” or “invariant TCR” refers to an invariant TCR that expresses on iNKT cells and includes but not limited to an invariant TCRα-chain and a limited number of non-invariant TCRβ-chains, paired with a limited repertoire of Vβ-chains (e.g., Vβ11 in humans) (e.g., Vα24-Jα18, Vα24-JαQ, Vα24).

As used herein, the term “genome editing” or “CRISPR-mediated genome editing” or “gene editing” or “genome engineering” refers to a type of genetic engineering in which DNA is deleted, inserted, modified, or replaced in the genome. Unlike lentiviral transduction to express a CAR that randomly inserts genetic material into a host genome, genome editing targets the insertions or deletions to site specific locations. The current nuclease-based genome editing tools include but are not limited to zinc finger nuclease (ZFN), transcription activator-like effector nuclease (TALEN), meganuclease, and CRISPR/Cas9.

In some embodiments, an NKT cell (e.g., INKT cell) is a single cell. In some embodiments, an NKT cell (e.g., INKT cell) is a homogenous cell population. In some embodiments, an NKT cell (e.g., iNKT cell) is a heterogenous cell population. In some embodiments, an NKT cell (e.g., iNKT cell) causes, stimulates, and/or contributes to the production of at least one cytokine (e.g., IL-4 and/or IFN-γ). In some embodiments, an NKT cell (e.g., iNKT cell) is cytotoxic. In some embodiments, an NKT cell (e.g., an iNKT cell) may display cytotoxicity against various cells, including cancer cells or cell lines (e.g., T-ALL cells or cell lines), as described and exemplified herein.

In some embodiments, INKT cells of the present disclosure may express any number or combination of cell surface markers. For instance, in some embodiments, INKT cells may express CD3 and iTCR(Vα24-Jα18) or CD3 and Vα24 on the cell surface. In some embodiments, the CD3 and iTCR(Vα24-Jα18) cell surface markers may be expressed on their own, or in combination with one or more additional cell surface markers (e.g., CD4⁺, CD8⁺, CD4⁻CD8⁻, CD4⁺CD8⁺ etc.).

In some embodiments, INKT cells of the present disclosure (e.g., CD3⁺iTCR(Vα24-Jα18)⁺ INKT cells) may be obtained or isolated from a biological sample. In some embodiments, INKT cells of the present disclosure may be obtained or isolated from one or more peripheral blood mononuclear cells.

In some embodiments, a biological sample is from a human (e.g., a fetal, neonatal, child, or adult human). In some embodiments, the biological sample is from a non-human animal. Non-human animals include all vertebrates (e.g., mammals and non-mammals) such as mice, rats, rabbits, dogs, monkeys, and pigs. In some embodiments, the biological sample is from a subject in need of treatment (e.g., a cancer patient, e.g., a T-ALL patient). In some embodiments, the biological sample is from a donor (e.g., a healthy donor). In some embodiments, the biological sample comprises blood (e.g., peripheral blood and/or umbilical cord blood), bone marrow, lymph node tissue, spleen tissue, tumor tissue, one or more induced pluripotent stem cells, and/or one or more peripheral blood mononuclear cells.

In some embodiments, the biological sample and/or blood comprises peripheral blood and/or umbilical cord blood. In some embodiments, the biological sample and/or blood is collected (e.g., from a subject or donor) by apheresis and/or leukapheresis.

In some embodiments, iNKT cells of the present disclosure (e.g., CD3⁺iTCR(Vα24-Jα18)⁺ INKT cells) may be isolated, e.g., from a human biological sample. In some embodiments, the INKT cells are isolated INKT cells.

As used herein, the term “isolated” refers to a material that is removed from its source environment (e.g., the natural environment if it is naturally-occurring). For example, a naturally-occurring polynucleotide, polypeptide, or cell present in a living organism is not isolated, but the same polynucleotide, polypeptide, or cell separated from some or all of the coexisting materials in the living organism, is isolated.

An “isolated cell,” as used herein, refers to a cell or cell population (e.g., a type I NKT cell or cell population) that has been identified and separated from one or more (e.g., the majority) of the components of its source environment (e.g., from the components of a cell culture or a biological sample). In some embodiments, the separation is performed such that it sufficiently removes components that may otherwise interfere with the suitability of the cell for the desired applications (e.g., for therapeutic use of a type I NKT cell or cell population). In some embodiments, the separation is performed such that it sufficiently separates cells expressing a particular marker or set of markers (e.g., iTCR(Vα24-Jα18)) from cells expressing an alternate marker or set of markers. Methods for isolating cells are known in the art and include, without limitation, separation by positive and/or negative selection techniques, or by cell sorting, for example, using antibody-conjugated microbeads, using flow cytometry with a cocktail of monoclonal antibodies directed to cell surface markers, etc. Exemplary isolation and separation techniques are described and exemplified herein.

In some embodiments, iNKT cells of the present disclosure (e.g., CD3⁺iTCR(Vα24-Jα18)⁺ INKT cells) may be isolated or separated via affinity-based separation methods. Exemplary techniques for affinity separation may include, in some embodiments, magnetic separation (e.g., using antibody-coated magnetic beads), affinity chromatography, cytotoxic agents joined to a monoclonal antibody or use in conjunction with a monoclonal antibody (e.g., complement and cytotoxins), and “panning” with an antibody attached to a solid matrix (e.g., a plate), or any other suitable technique. In some embodiments, separation techniques may also include the use of fluorescence activated cell sorters, which can have varying degrees of sophistication, such as multiple color channels, low angle and obtuse light scattering detecting channels, impedance channels, etc. It is to be understood that any technique that enables isolation or separation of INKT cells (e.g., iTCR(Vα24-Jα18)⁺ INKT cells) may be employed.

In some embodiments, affinity reagents employed in various isolation or separation methods may be specific receptors or ligands for cell surface markers on the INKT cells. In some embodiments, antibodies may be conjugated to a label, which may, in some embodiments, be used for isolation or separation. Labels may include, in some embodiments, magnetic beads (e.g., which may allow for direct separation), biotin (e.g., which may be removed with avidin or streptavidin bound to, e.g., a support; e.g., biotin conjugated anti-TCRVα24-Jα18 (6B11 clone) or anti-Vα24 antibodies in combination with streptavidin microbeads), fluorochromes (e.g., which may be used with a fluorescence activated cell sorter, e.g., phycoerythrin, fluorescein, Texas red, or a combination thereof), or the like.

In some embodiments, cell separations utilizing antibodies may comprise the addition of an antibody to a suspension of cells, e.g., for a period of time sufficient to bind available cell surface markers. The incubation may be for a varied period of time. For example, in some embodiments, the incubation may be for about 2 minutes, about 5 minutes, about 15 minutes, about 30 minutes, about 45 minutes, about 60 minutes, about 90 minutes, or longer. Any length of time which results in specific labeling with the antibody, with minimal non-specific binding, may be considered envisioned for this aspect of the disclosure.

In some embodiments, staining intensity of INKT cells can be monitored by flow cytometry, for example, where lasers detect quantitative levels of a fluorochrome (which may be proportional to the amount of cell surface antigen bound by antibodies). Flow cytometry, or FACS, can also be used, in some embodiments, to separate cell populations based on the intensity of antibody staining, as well as other parameters such as cell size and light scatter.

In some embodiments, INKT cells are separated based on their expression of at least one cell surface marker. The separated cells may be collected in any appropriate medium that maintains cell viability. In some embodiments, a culture containing the cells may contain serum, cytokines, or growth factors to which the cells are responsive. In some embodiments, a cytokine or growth factor may promote cell survival, growth, function, or a combination thereof. Cytokines and growth factors may include, in some embodiments, polypeptides and non-polypeptide factors.

As used herein, the term “activated” refers to a process of making isolated INKT cells or conventional T cells active (e.g., proliferative, releasing cytokines or cytolytic) In some embodiments, isolated iNKT cells or conventional T cells are activated by co-culture with T cell receptor (TCR) engaging reagents (e.g., microbeads coated with antibodies to CD2, CD3, and CD28 or soluble anti-CD3 antibody). In some embodiments, isolated INKT cells are activated by co-culture in human T-cell medium with irradiated autologous PBMC negative fraction, αGalCer or 7DW8-5, IL-2, IL-15, and/or IL-7.

In some embodiments, iNKT cells of the present disclosure (e.g., CD3⁺iTCR(Vα24-Jα18)⁺ INKT cells) are modified to express a CD7 CAR construct capable of binding to CD7 target antigen. In some embodiments, CD7 CAR-modified INKT cells of the present disclosure are further modified to express a chimeric antigen receptor (CAR), a T cell receptor (TCR), a T cell receptor mimic antibody (TCRm), or any combination thereof to target additional antigens on a CD7⁺ cancer.

In some embodiments, INKT cells are modified to express a chimeric antigen receptor (CAR). In some embodiments, a CAR can be engineered using an antigen binding domain such that when the CAR is expressed on a cell (e.g., an INKT cell), the CAR and/or cell binds to the target antigen (e.g., CD7 or another exemplary antigen described herein). In some embodiments, the CAR sequences are cloned into a cell or cell population (e.g., an INKT cell or iNKT cell population) and expanded using our own protocol of irradiated allogeneic PBMCs and EBV-B cells, αGalCer or 7DW8-5, and mixed cytokines (IL-2, IL-15 or IL-2, IL-7, and IL-15) and/or currently available protocols. In some embodiments, the cell or cell population comprises one or more polynucleotides encoding the CAR. In some embodiments, the cell or cell population is from a donor or a patient (e.g., a patient having or suspected of having a cancer, e.g., T-ALL or another exemplary cancer described herein).

In some embodiments, when used as a therapeutic agent, and when the cell or cell population is from a patient, the CAR-modified cell or cell population may be administered to the same patient and/or to another patient in need of such treatment. In some embodiments, when used as a therapeutic agent, and when the cell or cell population is from a donor, the CAR-modified cell or cell population may be administered to any patient in need of such treatment.

As used herein, the term “CAR-expressing” and “CAR-modified” when used to describe a cell or cell population refers to a cell or cell population that has been artificially engineered to comprise one or more polynucleotides encoding the sequence of a CAR peptide and which can transcribe, translate, and express the CAR peptide on the cell surface. In some embodiments, the CAR-expressing cell or cell population comprises an INKT cell or cell population. In some embodiments, the CAR-expressing cell or cell population comprises a iTCR(Vα24-Jα18)⁺ INKT cell or cell population. In some embodiments, the CAR-expressing cell or cell population comprises a CD3⁺iTCR(Vα24-Jα18)⁺ INKT cell or cell population. In some embodiments, the CAR-expressing cell or cell population comprises a CD3⁺iTCR(Vα24-Jα18)⁺CD4⁺ cell or cell population. In some embodiments, the CAR-expressing cell or cell population comprises a CD3⁺iTCR(Vα24-Jα18)⁺CD4⁻CD8⁻ cell or cell population. In some embodiments, the CAR-expressing cell or cell population comprises a CD3⁺iTCR(Vα24-Jα18)⁺CD8⁺ cell or cell population. In some embodiments, the CAR-expressing cell or cell population comprises a CD3⁺iTCR(Vα24-Jα18)⁺CD4⁺CD8⁺ cell or cell population. In some embodiments, the CAR-expressing cell or cell population comprises a mixture of cells, e.g., a mixture of CD3⁺iTCR(Vα24-Jα18)⁺CD4⁺ and CD3⁺iTCR(Vα24-Jα18)⁺CD8⁺ cells, a mixture of CD3⁺iTCR(Vα24-Jα18)⁺CD4⁺ and CD3⁺iTCR(Vα24-Jα18)⁺CD4⁻CD8⁻ cells, a mixture of CD3⁺iTCR(Vα24-Jα18)⁺CD4⁺ and CD3⁺iTCR(Vα24-Jα18)⁺CD4⁺CD8⁺ cells or a mixture of CD3⁺iTCR(Vα24-Jα18)⁺CD4⁺, CD3⁺iTCR(Vα24-Jα18)⁺CD8⁺, CD3⁺iTCR(Vα24-Jα18)⁺CD4⁺CD8⁻, and CD3⁺iTCR(Vα24-Jα18)⁺CD4⁺CD8⁺ cells. In some embodiments, when used as a therapeutic agent, the CAR-expressing cell or cell population administered to a subject may comprise a CAR-modified NKT cell, or a population of CAR-modified NKT cells, from the subject. In some embodiments, when used as a therapeutic agent, the CAR-expressing cell or cell population administered to a subject may comprise a CAR-modified NKT cell, or a population of CAR-modified NKT cells, from a donor.

In some embodiments, a CAR-modified cell or cell population can engage with and kill cells (e.g., malignant cancer cells) that express the target antigen (e.g., CD7). Methods and compositions for making and administering the disclosed CAR-based immunotherapies are provided herein. Exemplary methods for making CAR-based immunotherapies are also disclosed in, e.g., U.S. Publication Nos. 2014/0271635 and 2016/0310532, which are both incorporated herein by reference for such methods.

The terms “chimeric antigen receptor” and “CAR,” as used herein, refer to a polypeptide or a set of polypeptides, which, when expressed by a cell, provide the cell with specificity for a target antigen-expressing cell (e.g., a malignant cancer cell) and with intracellular signal generation. In some embodiments, a CAR comprises at least an extracellular antigen binding domain, a hinge domain, a transmembrane domain, and an intracellular signaling domain. In some embodiments, a CAR comprises at least an extracellular antigen binding domain, a hinge domain, a transmembrane domain, and an intracellular signaling domain comprising a functional signaling domain derived from a stimulatory molecule and/or a costimulatory molecule. These domains may reside in a single polypeptide or a set of polypeptides. In some embodiments, the stimulatory molecule is the zeta chain associated with the T cell receptor complex. In some embodiments, the costimulatory molecule is 4-1BB, CD28, CD27, CD134 (OX40), ICOS, DAP10, and/or DAP12.

In some embodiments, a CAR comprises a chimeric fusion protein comprising an extracellular antigen binding domain, a hinge domain, a transmembrane domain, and an intracellular signaling domain comprising: (i) a functional signaling domain derived from a stimulatory molecule; (ii) a functional signaling domain derived from a stimulatory molecule and a functional signaling domain derived from a costimulatory molecule; or (iii) a functional signaling domain derived from a stimulatory molecule and at least two functional signaling domains derived from one or more costimulatory molecule(s). In some embodiments, a CAR comprises an optional leader sequence at the N-terminus of the CAR fusion protein. In some embodiments, a CAR further comprises a leader sequence at the N-terminus of the extracellular antigen binding domain, wherein the leader sequence is optionally cleaved from the antigen binding domain during cellular processing and localization of the CAR to the cellular membrane.

In some embodiments, an antigen binding domain of a CAR comprises an antibody or an antigen binding fragment thereof. In some embodiments, the antigen binding domain and/or antibody comprises a monoclonal antibody, a polyclonal antibody, a synthetic antibody, a human antibody, a humanized antibody, or a single domain antibody (also known as a nanobody). In some embodiments, the antigen binding domain and/or antigen binding fragment comprises a single chain variable fragment (scFv) or a Fab fragment. In some embodiments, the antigen binding domain and/or antigen binding fragment comprises an scFv.

As used herein, the term “antibody” refers to any functional immunoglobulin molecule that recognizes and binds, e.g., specifically binds, to a target, such as a protein, polypeptide, carbohydrate, polynucleotide, lipid, or combinations of the foregoing through at least one antigen recognition site within the variable region of the immunoglobulin molecule. The term “antibody” encompasses antibodies having sequences from any source species, such as mouse, rabbit, goat, llama, alpaca, non-human primate, and human. The term further encompasses human antibodies, chimeric antibodies, humanized antibodies, and any modified immunoglobulin molecule containing an antigen recognition site, so long as it demonstrates the desired binding and/or biological activity. In some embodiments, an antibody possesses the ability to bind, e.g., specifically bind, a target antigen expressed on a cancer cell (e.g., CD7). An antibody can be generated using any suitable technology, e.g., recombinant expression, hybridoma technology, ribosome display, phage display, gene shuffling libraries, semi-synthetic, or fully synthetic libraries, or any combination thereof. The term “antibody” includes full-length antibodies as well as antigen binding domains and antigen binding fragments thereof. In some embodiments, an antibody used in the CARs and/or other constructs described herein is a full-length or intact antibody. In some embodiments, an antibody used in the CARs and/or other constructs described herein is a monoclonal antibody, a polyclonal antibody, a synthetic antibody, a human antibody, a humanized antibody, or a single domain antibody. In some embodiments, an antibody used in the CARs and/or other constructs described herein is an antigen binding domain or an antigen binding fragment of an antibody.

A “full-length” or “intact” antibody typically comprises at least two heavy (H) chains and two light (L) chains inter-connected by disulfide bonds. The recognized classes of immunoglobulin genes encoding antibody chains include the kappa, lambda, alpha, gamma, delta, epsilon, and mu constant region genes, as well as the myriad immunoglobulin variable region genes. Light chains are classified as either kappa or lambda. In some embodiments, an antibody comprises a kappa light chain. In some embodiments, an antibody comprises a lambda light chain. The kappa or lambda light chain may be selected from any kappa or lambda light chain sequence from any species. Heavy chains are classified as gamma, mu, alpha, delta, or epsilon, which in turn define the immunoglobulin classes, IgG, IgM, IgA, IgD and IgE, respectively. The four subclasses of IgG (IgG1, IgG2, IgG3, and IgG4) differ in their constant region and exhibit different effector functions.

Antibodies that may be used in the CARs and/or other constructs described herein also include antigen binding fragments. The term “antigen binding fragment” or “antigen binding portion” of an antibody, as used herein, refers to one or more fragments of a full-length antibody that retain the ability to bind, e.g., specifically bind, to the target antigen (e.g., CD7) and/or provide a function of the full-length antibody (e.g., the ability to specifically bind to CD7). Antigen binding functions of an antibody can be performed by fragments of a full-length antibody. Fragments can also be present in larger macromolecules, e.g., bispecific antibodies. Examples of such antibody fragments include a Fab fragment, a monovalent fragment comprising at least a VL, CL, VH, and CH1 domain; a F (ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (SdAb) fragment, which consists of a VH domain or a VL domain; an isolated complementarity determining region (CDR); and a half body, which comprises only one heavy chain and one light chain rather than the typical pairing of two heavy and two light chains on separate arms. Furthermore, although the two domains of the Fv fragment, VL and VH, are coded for by separate genes, they can be joined using recombinant methods, e.g., by an artificial peptide linker that enables them to be made as a single protein chain in which the VL and VH regions pair to form monovalent molecules (known as a single chain variable fragment (scFv)) (see, e.g., Bird et al., Science 1988; 242 (4877): 423-6; Huston et al., PNAS 1988; 85 (16): 5879-83). Such single chain antibodies include one or more antigen binding fragments or portions of an antibody. Other examples of antigen binding fragments include bispecific T cell engagers (BiTEs), which consist of two scFvs of different antibodies, or amino acid sequences from four different genes, on a single peptide chain. In some embodiments, an antigen binding fragment is a Fab fragment or an scFv. In some embodiments, an antigen binding fragment is an scFv.

In some embodiments, an antigen binding domain of a CAR comprises a cell-binding agent. In some embodiments, an antigen binding domain and/or cell-binding agent of a CAR comprises a DARPin, duobody, bicyclic peptide, nanobody, centyrin, MSH (melanocyte-stimulating hormone), receptor-Fc fusion molecule, T cell receptor structure, natural ligand (e.g., a receptor expressed in mature non-malignant and/or malignant B cells, including plasma cells, e.g., exemplary ligands to B-cell maturation antigen (BCMA) include, without limitation, B-cell activating factor (BAFF) and proliferation inducing ligand (APRIL)), steroid hormone (e.g., an androgen or estrogen), growth factor, colony-stimulating factor (e.g., EGF), or other non-antibody scaffold. In some embodiments, non-antibody scaffolds can broadly fall into two structural classes, namely domain-sized compounds (approximately 6-20 kDa) and constrained peptides (approximately 2-4 kDa). Exemplary domain-sized scaffolds include but are not limited to affibodies, affilins, anticalins, atrimers, DARPins, FN3 scaffolds (e.g., adnectins and centyrins), fynomers, Kunitz domains, pronectins, O-bodies, and receptor-Fc fusion proteins, whereas exemplary constrained peptides include avimers, bicyclic peptides, and Cys-knots. In some embodiments, an antigen binding domain and/or cell-binding agent of a CAR comprises an affibody, an affilin, an anticalin, an atrimer, a DARPin, a FN3 scaffold such as an adnectin or a centyrin, a fynomer, a Kunitz domain, a pronectin, an O-body, a receptor-Fc fusion protein, an avimer, a bicyclic peptide, and/or a Cys-knot. Non-antibody scaffolds are reviewed, e.g., in Vazquez-Lombardi et al., Drug Dis Today 2015; 20 (10): 1271-83.

In some embodiments, an antigen binding domain of a CAR is capable of binding to CD7, CD1a, CD1d, CD5, TRBC1, TRBC2, CD21, CCR9, CD30, CD123, CD33, CD38, CD138, CLL-1, LILRB4, Siglec-6, CD70, or PD-L1. In some embodiments, the antigen binding domain is capable of binding to CD19 or ROR1.

The terms “CD7, CD1a, CD1d, CD5, TRBC1, CD21, CCR9, CD123, CD33, CD38, CD138, CLL-1, LILRB4, CD30, Siglec-6, CD70, PD-L1, CD19, or ROR1”, refers to any native form of the antigens (e.g., CD7). The term encompasses full-length CD7 (e.g., UniProt Reference Sequence: P09564; SEQ ID NO: 1), CD1a (e.g., UniProt Reference Sequence: P06126; SEQ ID NO: 2), CD1d (e.g., UniProt Reference Sequence: P15813; SEQ ID NO: 3), CD2 (e.g., UniProt Reference Sequence: P06729; SEQ ID NO: 4), CD5 (e.g., UniProt Reference Sequence: P06127; SEQ ID NO: 5), TRBC1 (e.g., UniProt Reference Sequence: P01850; SEQ ID NO: 6), TRBC2 (e.g., UniProt Reference Sequence: A0A5B9; SEQ ID NO: 7), CD21 (e.g., UniProt Reference Sequence: P20023; SEQ ID NO: 8), CCR9 (e.g., UniProt Reference Sequence: P51686; SEQ ID NO: 9), CD30 (e.g., UniProt Reference Sequence: P28908; SEQ ID NO: 10), CD123 (e.g., UniProt Reference Sequence: P26951; SEQ ID NO: 11), CD33 (e.g., UniProt Reference Sequence: P20138; SEQ ID NO: 12), CD38 (e.g., UniProt Reference Sequence: P28907; SEQ ID NO: 13), CD138 (e.g., UniProt Reference Sequence: P18827; SEQ ID NO: 14), CLL-1 (e.g., UniProt Reference Sequence: Q5QGZ9; SEQ ID NO: 15), LILRB4 (e.g., UniProt Reference Sequence: Q8NHJ6; SEQ ID NO: 16), Siglec-6 (e.g., UniProt Reference Sequence: 043699; SEQ ID NO: 17), CD70 (e.g., UniProt Reference Sequence: P32970; SEQ ID NO. 18), PD-L1 (e.g., UniProt Reference Sequence: Q9NZQ7; SEQ ID NO: 19), CD19 (e.g., UniProt Reference Sequence: P15391; SEQ ID NO: 20), ROR1 (e.g., UniProt Reference Sequence: Q01973; SEQ ID NO: 21) or any antigens, as well as any form of the antigens (e.g., CD7) that may result from cellular processing. The term also encompasses functional variants or fragments of the antigens, including but not limited to splice variants, allelic variants, and isoforms that retain one or more biologic functions of the antigens (i.e., variants and fragments are encompassed unless the context indicates that the term is used to refer to the wild-type protein only). The antigens (e.g., CD7) can be isolated from a human, or may be produced recombinantly or by synthetic methods.

In some embodiments, CD7 CAR-modified INKT cells are modified to express a T cell receptor (TCR). In some embodiments, a TCR can be engineered using an antigen binding alpha chain and/or beta chain such that when the TCR is expressed on a cell (e.g., CD7 CAR-modified INKT cell), the TCR and/or cell binds to the target antigen (e.g., WT1 or another exemplary antigen described herein). In some embodiments, the TCR sequences are cloned into a cell or cell population (e.g., CD7 CAR-modified INKT cell or INKT cell population) and expanded using our own protocol of irradiated allogeneic PBMCs and EBV-B cells, αGalCer or 7DW8-5, and mixed cytokines (IL-2, IL-15 or IL-2, IL-7, and IL-15) and/or currently available protocols. In some embodiments, the cell or cell population comprises one or more polynucleotides encoding the TCR. In some embodiments, the cell or cell population is from a donor or a patient (e.g., a patient having or suspected of having a cancer, e.g., T-ALL or another exemplary cancer described herein). In some embodiments, when used as a therapeutic agent, and when the cell or cell population is from a patient, the TCR-modified cell or cell population may be administered to the same patient and/or to another patient in need of such treatment. In some embodiments, when used as a therapeutic agent, and when the cell or cell population is from a donor, the TCR-modified cell or cell population may be administered to any patient in need of such treatment.

As used herein, the term “TCR-expressing” and “TCR-modified” when used to describe a cell or cell population refers to a cell or cell population that has been artificially engineered to comprise one or more polynucleotides encoding the sequence of a TCR peptide and which can transcribe, translate, and express the TCR peptide on the cell surface. In some embodiments, the TCR-expressing cell or cell population comprises a CD7 CAR-modified INKT cell or cell population. In some embodiments, the TCR-expressing cell or cell population comprises a CD3⁺iTCR(Vα24-Jα18)⁺CD4⁺ cell or cell population. In some embodiments, the TCR-expressing cell or cell population comprises a CD3⁺iTCR(Vα24-Jα18)⁺CD4⁻CD8⁻ cell or cell population. In some embodiments, the TCR-expressing cell or cell population comprises a CD3⁺iTCR(Vα24-Jα18)⁺CD8⁺ cell or cell population. In some embodiments, the TCR-expressing cell or cell population comprises a CD3⁺iTCR(Vα24-Jα18)⁺CD4⁺CD8⁺ cell or cell population. In some embodiments, the TCR-expressing cell or cell population comprises a mixture of cells, e.g., a mixture of CD3⁺iTCR(Vα24-Jα18)⁺CD4⁺ and CD3⁺iTCR(Vα24-Jα18)⁺CD8⁺ cells, a mixture of CD3⁺iTCR(Vα24-Jα18)⁺CD4⁺ and CD3⁺iTCR(Vα24-Jα18)⁺CD4⁻CD8⁻ cells, a mixture of CD3⁺iTCR(Vα24-Jα18)⁺CD4⁺ and CD3⁺iTCR(Vα24-Jα18)⁺CD4⁺CD8⁺ cells or a mixture of CD3⁺iTCR(Vα24-Jα18)⁺CD4⁺, CD3⁺iTCR(Vα24-Jα18)⁺CD8⁺, CD3⁺iTCR(Vα24-Jα18)⁺CD4⁻CD8⁻, and CD3⁺iTCR(Vα24-Jα18)⁺CD4⁺CD8⁺ cells. In some embodiments, when used as a therapeutic agent, the TCR-expressing cell or cell population administered to a subject may comprise a TCR-modified NKT cell, or a population of TCR-modified NKT cells, from the subject. In some embodiments, when used as a therapeutic agent, the TCR-expressing cell or cell population administered to a subject may comprise a TCR-modified NKT cell, or a population of TCR-modified NKT cells, from a donor.

In some embodiments, a TCR-modified cell or cell population can engage with and kill cells (e.g., malignant cancer cells) that express the target antigen (e.g., WT1). Methods and compositions for making and administering the disclosed TCR-based immunotherapies are provided herein. Exemplary methods for making TCR-based immunotherapies are also disclosed in, e.g., U.S. Pat. No. 9,115,372, which is incorporated herein by reference for such methods.

The terms “T cell receptor” and “TCR,” as used herein, refer to a polypeptide or a set of polypeptides, which, when expressed by a cell, provide the cell with specificity for a target antigen-expressing cell (e.g., a malignant cancer cell) and with intracellular signal generation. In some embodiments, a TCR comprises at least an alpha chain and a beta chain. These chains may reside in a single polypeptide or a set of polypeptides. In some embodiments, the alpha chain and/or the beta chain is capable of binding to an antigen.

In some embodiments, the TCR comprises an alpha chain and a beta chain. In some embodiments, both the alpha chain and the beta chain comprise a constant region (c) and a variable region (v). In some embodiments, the variable region determines antigen specificity. In some embodiments, the variable region recognizes a target antigen, e.g., an antigen ligand comprising a short contiguous amino acid sequence of a protein that is presented on the target cell by a major histocompatibility complex (MHC) molecule (also known as a human leukocyte antigen (HLA) molecule). In some embodiments, accessory adhesion molecules expressed by T cells, such as CD4 for MHC class II and CD8 for MHC class I, are also involved. In some embodiments, signal transduction of a TCR is through an associated invariant CD3 complex. In some embodiments, the CD3 complex comprises different CD3 proteins that form two heterodimers (CD308 and CD3γε) and one homodimer (CD3ξξ).

In some embodiments, an alpha chain and/or a beta chain of a TCR is capable of binding to an antigen. In some embodiments, the alpha chain and/or beta chain is capable of binding to WT1, PRAME, HA-1 or ΔNPM1.

The term “WT1, PRAME, HA-1, and ΔNPM1,” as used herein, refers to any native form of human WT1, PRAME, HA-1, and ΔNPM1. The term encompasses full-length WT1 (e.g., UniProt Reference Sequence: P19544; SEQ ID NO: 22), PRAME (e.g., UniProt Reference Sequence: P78395; SEQ ID NO: 23), HA-1 (e.g., UniProt Reference Sequence: Q92619; SEQ ID NO: 24), and ΔNPM1 (e.g., UniProt Reference Sequence: P06748; SEQ ID NO: 25), as well as any form of human WT1, PRAME, HA-1, and ΔNPM1 that may result from cellular processing. The term also encompasses functional variants or fragments of WT1, PRAME, HA-1, and ΔNPM1, including but not limited to splice variants, allelic variants, and isoforms that retain one or more biologic functions of human WT1, PRAME, HA-1, and ΔNPM1 (i.e., variants and fragments are encompassed unless the context indicates that the term is used to refer to the wild-type protein only). WT1, PRAME, HA-1, and ΔNPM1 can be isolated from a human, or may be produced recombinantly or by synthetic methods.

In some embodiments, CD7 CAR-expressing INKT cells are further modified to express a T cell receptor mimic antibody (TCRm). In some embodiments, a TCRm can be engineered using an antigen binding domain such that when the TCRm is expressed on a cell (e.g., an iNKT cell), the TCRm and/or cell binds to the target antigen (e.g., a composite antigen, e.g., a composite antigen comprising a peptide and a human leukocyte antigen (HLA) molecule, as described herein). In some embodiments, the TCRm sequences are cloned into a cell or cell population (e.g., an iNKT cell or iNKT cell population) and expanded using our own protocol of irradiated allogeneic PBMCs and EBV-B cells, αGalCer or 7DW8-5, and mixed cytokines (IL-2, IL-15 and/or IL-7) and/or currently available protocols. In some embodiments, the cell or cell population comprises one or more polynucleotides encoding the TCRm. In some embodiments, the cell or cell population is from a donor or a patient (e.g., a patient having or suspected of having a cancer, e.g., T-ALL or another exemplary cancer described herein). In some embodiments, when used as a therapeutic agent, and when the cell or cell population is from a patient, the TCRm-modified cell or cell population may be administered to the same patient and/or to another patient in need of such treatment. In some embodiments, when used as a therapeutic agent, and when the cell or cell population is from a donor, the TCRm-modified cell or cell population may be administered to any patient in need of such treatment.

As used herein, the term “TCRm-expressing” and “TCRm-modified” when used to describe a cell or cell population refers to a cell or cell population that has been artificially engineered to comprise one or more polynucleotides encoding the sequence of a TCRm peptide and which can transcribe, translate, and express the TCRm peptide on the cell surface. In some embodiments, the TCRm-expressing cell or cell population comprises a CD7 CAR-modified INKT cell or cell population. In some embodiments, the TCRm-expressing cell or cell population comprises a CD3⁺iTCR(Vα24-Jα18)⁺CD4⁺ cell or cell population. In some embodiments, the TCRm-expressing cell or cell population comprises a CD3⁺iTCR(Vα24-Jα18)⁺CD4⁻CD8⁻ cell or cell population. In some embodiments, the TCRm-expressing cell or cell population comprises a CD3⁺iTCR(Vα24-Jα18)⁺CD8⁺ cell or cell population. In some embodiments, the TCRm-expressing cell or cell population comprises a mixture of cells, e.g., a mixture of CD3⁺iTCR(Vα24-Jα18)⁺CD4⁺ and CD3⁺iTCR(Vα24-Jα18)⁺CD8⁺ cells, a mixture of CD3⁺iTCR(Vα24-Jα18)⁺CD4⁺ and CD3⁺iTCR(Vα24-Jα18)⁺CD4⁻CD8⁻ cells, a mixture of CD3⁺iTCR(Vα24-Jα18)⁺CD4⁺ and CD3⁺iTCR(Vα24-Jα18)⁺CD4⁺CD8⁺ cells or a mixture of CD3⁺iTCR(Vα24-Jα18)⁺CD4⁺, CD3⁺iTCR(Vα24-Jα18)⁺CD8⁺, CD3⁺iTCR(Vα24-Jα18)⁺CD4⁻CD8⁻, and CD3⁺iTCR(Vα24-Jα18)⁺CD4⁺CD8⁺ cells. In some embodiments, when used as a therapeutic agent, the TCRm-expressing cell or cell population administered to a subject may comprise a TCRm-modified NKT cell, or a population of TCRm-modified NKT cells, from the subject. In some embodiments, when used as a therapeutic agent, the TCRm-expressing cell or cell population administered to a subject may comprise a TCRm-modified NKT cell, or a population of TCRm-modified NKT cells, from a donor.

In some embodiments, a TCRm-modified cell or cell population can engage with and kill cells (e.g., malignant cancer cells) that express the target antigen (e.g., a composite antigen, e.g., a composite antigen comprising a peptide and a human leukocyte antigen (HLA) molecule, as described herein). Methods and compositions for making and administering the disclosed TCRm-based immunotherapies are provided herein. Exemplary methods for making TCRm-based immunotherapies are also disclosed in, e.g., U.S. Publication No. 2019/092876, which is incorporated herein by reference for such methods.

The terms “T cell receptor mimic antibody” and “TCRm,” as used herein, refer to a polypeptide or a set of polypeptides, which, when expressed by a cell, provide the cell with specificity for a target antigen-expressing cell (e.g., a malignant cancer cell) and with intracellular signal generation. In some embodiments, a TCRm comprises at least an extracellular antigen binding domain, a hinge domain, a transmembrane domain, and an intracellular signaling domain. In some embodiments, a TCRm comprises at least an extracellular antigen binding domain, a hinge domain, a transmembrane domain, and an intracellular signaling domain comprising a functional signaling domain derived from a stimulatory molecule and/or a costimulatory molecule. These domains may reside in a single polypeptide or a set of polypeptides. In some embodiments, the stimulatory molecule is the zeta chain associated with the T cell receptor complex. In some embodiments, the costimulatory molecule is 4-1BB, CD28, CD27, CD134 (OX40), ICOS, DAP10, and/or DAP12.

In some embodiments, an antigen binding domain of a TCRm comprises an antibody or an antigen binding fragment thereof. In some embodiments, the antigen binding domain and/or antibody comprises a monoclonal antibody, a polyclonal antibody, a synthetic antibody, a human antibody, a humanized antibody, or a single domain antibody. In some embodiments, the antigen binding domain and/or antigen binding fragment comprises a single chain variable fragment (scFv) or a Fab fragment. In some embodiments, the antigen binding domain and/or antigen binding fragment comprises an scFv.

In some embodiments, an antigen binding domain of a TCRm comprises a cell-binding agent. In some embodiments, an antigen binding domain and/or cell-binding agent of a TCRm comprises a DARPin, duobody, bicyclic peptide, nanobody, centyrin, MSH (melanocyte-stimulating hormone), receptor-Fc fusion molecule, T cell receptor structure, natural ligand (e.g., a receptor expressed in mature non-malignant and/or malignant B cells, including plasma cells, e.g., exemplary ligands to B-cell maturation antigen (BCMA) include, without limitation, B-cell activating factor (BAFF) and proliferation inducing ligand (APRIL)), steroid hormone (e.g., an androgen or estrogen), growth factor, colony-stimulating factor (e.g., EGF), or other non-antibody scaffold. In some embodiments, an antigen binding domain and/or cell-binding agent of a TCRm comprises an affibody, an affilin, an anticalin, an atrimer, a DARPin, a FN3 scaffold such as an adnectin or a centyrin, a fynomer, a Kunitz domain, a pronectin, an O-body, a receptor-Fc fusion protein, an avimer, a bicyclic peptide, and/or a Cys-knot.

In some embodiments, an antigen binding domain of a TCRm is capable of binding to a composite antigen comprising a peptide and a human leukocyte antigen (HLA) molecule.

In some embodiments, the HLA molecule is a class I HLA molecule. In some embodiments, the HLA molecule is a class I HLA binding peptide. In some embodiments, a class I HLA binding peptide is about 9 or 10 amino acids in length. Exemplary class I HLA binding peptides include but are not limited to: WT1-derived HLA-A*0201 binding peptide RMFPNAPYL (SEQ ID NO: 26); WT1-derived HLA-A*2402 binding peptide CMTWNQMNL (SEQ ID NO: 27); PRAME-derived HLA-A*0201 binding peptide VLDGLDVLL (SEQ ID NO: 28); PRAME-derived HLA-A*0201 binding peptide ALYVDSLEFL (SEQ ID NO: 29); PRAME-derived HLA-A*0201 binding peptide SLYSFPEPEA (SEQ ID NO: 30; PRAME-derived HLA-A*0201 binding peptide SLLQHLIGL (SEQ ID NO: 31); PRAME-derived HLA-A*2402 binding peptide LYVDSLFFLC (SEQ ID NO: 32); HA-1-derived HLA-A*0201 binding peptide VLHDDLLEA (SEQ ID NO: 33); ΔNPM1-derived HLA-A*0201 binding peptide CLAVEEVSL (SEQ ID NO: 34).

In some embodiments, an antigen binding domain of a TCRm is capable of binding to a composite antigen comprising an HLA molecule having an amino acid sequence of RMFPNAPYL (SEQ ID NO: 26); CMTWNQMNL (SEQ ID NO: 27); VLDGLDVLL (SEQ ID NO: 28); ALYVDSLEFL (SEQ ID NO: 29); SLYSFPEPEA (SEQ ID NO: 30); SLLQHLIGL (SEQ ID NO: 31); LYVDSLFFLc (SEQ ID NO: 32); VLHDDLLEA (SEQ ID NO: 33); and/or CLAVEEVSL (SEQ ID NO: 34).

In some embodiments, the HLA molecule is a class II HLA molecule. In some embodiments, the HLA molecule is a class II HLA binding peptide. In some embodiments, a class II HLA binding peptide is about 13 to about 25 amino acids in length. Exemplary class II HLA binding peptides include but are not limited to: WT1-derived HLA-DRB1*0405, -DRB1*1501, -DRB1*1502, -DPB1*0501 and -DPB1*0901 binding peptide KRYFKLSHLQMHSRKH (SEQ ID NO: 35).

In some embodiments, an antigen binding domain of a TCRm is capable of binding to a composite antigen comprising an HLA molecule having an amino acid sequence of KRYFKLSHLQMHSRKH (SEQ ID NO: 35).

In some embodiments, INKT cells of the present disclosure (e.g., CD3⁺iTCR(Vα24-Jα18)⁺ or CD3⁺Vα24⁺ INKT cells) are further modified to comprise an exogenous cytokine, growth factor, antibody or antigen binding fragment, or any combination thereof. In some embodiments, the antibody or antigen binding fragment comprises a bispecific T cell engager (BITE).

TABLE 1
Exemplary Target Antigen Amino Acid and ScFv Sequences

| | SEQ | |
|---|---|---|
| Antigen | ID NO | Amino Acid Sequence |
| CD7 | 1 | MAGPPRLLLLPLLLALARGLPGALAAQEVQQSPHCTTVPVGASVNIT |
| | | CSTSGGLRGIYLRQLGPQPQDIIYYEDGVVPTTDRRERGRIDFSGSQDNL |
| | | TITMHRLQLSDTGTYTCQAITEVNVYGSGTLVLVTEEQSQGWHRCSDAPP |
| | | RASALPAPPTGSALPDPQTASALPDPPAASALPAALAVISELLGLGLGVA |
| | | CVLARTQIKKLCSWRDKNSAACVVYEDMSHSRCNTLSSPNQYQ |
| CD1a | 2 | MLFLLLPLLAVLPGDGNADGLKEPLSFHVTWIASFYNHSWKQNLVSG |
| | | WLSDLQTHTWDSNSSTIVELCPWSRGNFSNEEWKELETLFRIRTIRSFEG |
| | | IRRYAHELQFEYPFEIQVTGGCELHSGKVSGSFLQLAYQGSDEVSFQNNS |
| | | WLPYPVAGNMAKHFCKVLNQNQHENDITHNLLSDTCPRFILGLLDAGKAH |
| | | LQRQVKPEAWLSHGPSPGPGHLQLVCHVSGFYPKPVWVMWMRGEQEQQGT |
| | | QRGDILPSADGTWYLRATLEVAAGEAADLSCRVKHSSLEGQDIVLYWEHH |
| | | SSVGFIILAVIVPLLLLIGLALWERKRCFC |
| CD1d | 3 | MGCLLFLLLWALLQAWGSAEVPQRLEPLRCLQISSFANSSWTRTDGL |
| | | AWLGELQTHSWSNDSDTVRSLKPWSQGTFSDQQWETLQHIERVYRSSFTR |
| | | DVKEFAKMLRLSYPLELQVSAGCEVHPGNASNNFFHVAFQGKDILSFQGT |
| | | SWEPTQEAPLWVNLAIQVLNQDKWTRETVQWLLNGTCPQFVSGLLESGKS |
| | | ELKKQVKPKAWLSRGPSPGPGRLLLVCHVSGFYPKPVWVKWMRGEQEQQG |
| | | TQPGDILPNADETWYLRATLDVVAGEAAGLSCRVKHSSLEGQDIVLYWGG |
| | | SYTSMGLIALAVLACLLFLLIVGFTSRFKRQTSYQGVL |
| CD2 | 4 | MSFPCKFVASFLLIFNVSSKGAVSKEITNALETWGALGQDINLDIPS |
| | | FQMSDDIDDIKWEKTSDKKKIAQFRKEKETFKEKDTYKLFKNGTLKIKHL |
| | | KTDDQDIYKVSIYDTKGKNVLEKIFDLKIQERVSKPKISWTCINTTLTCE |
| | | VMNGTDPELNLYQDGKHLKLSQRVITHKWTTSLSAKFKCTAGNKVSKESS |
| | | VEPVSCPEKGLDIYLIIGICGGGSLLMVEVALLVFYITKRKKQRSRRNDE |
| | | ELETRAHRVATEERGRKPHQIPASTPQNPATSQHPPPPPGHRSQAPSHRP |
| | | PPPGHRVQHQPQKRPPAPSGTQVHQQKGPPLPRPRVQPKPPHGAAENSLS |
| | | PSSN |
| CD5 | 5 | MPMGSLQPLATLYLLGMLVASCLGRLSWYDPDFQARLTRSNSKCQGQ |
| | | LEVYLKDGWHMVCSQSWGRSSKQWEDPSQASKVCQRLNCGVPLSLGPFLV |
| | | TYTPQSSIICYGQLGSFSNCSHSRNDMCHSLGLTCLEPQKTTPPTTRPPP |
| | | TTTPEPTAPPRLQLVAQSGGQHCAGVVEFYSGSLGGTISYEAQDKTQDLE |
| | | NFLCNNLQCGSFLKHLPETEAGRAQDPGEPREHQPLPIQWKIQNSSCTSL |
| | | EHCFRKIKPQKSGRVLALLCSGFQPKVQSRLVGGSSICEGTVEVRQGAQW |
| | | AALCDSSSARSSLRWEEVCREQQCGSVNSYRVLDAGDPTSRGLFCPHQKL |
| | | SQCHELWERNSYCKKVFVTCQDPNPAGLAAGTVASIILALVLLVVLLVVC |
| | | GPLAYKKLVKKFRQKKQRQWIGPTGMNQNMSFHRNHTATVRSHAENPTAS |
| | | HVDNEYSQPPRNSHLSAYPALEGALHRSSMQPDNSSDSDYDLHGAQRL |
| TRBC1 | 6 | DLNKVFPPEVAVFEPSEAEISHTQKATLVCLATGFFPDHVELSWWVN |
| | | GKEVHSGVSTDPQPLKEQPALNDSRYCLSSRLRVSATFWQNPRNHERCQV |
| | | QFYGLSENDEWTQDRAKPVTQIVSAEAWGRADCGFTSVSYQQGVLSATIL |
| | | YEILLGKATLYAVLVSALVLMAMVKRKDE |
| TRBC2 | 7 | DLKNVFPPKVAVFEPSEAEISHTQKATLVCLATGFYPDHVELSWWVN |
| | | GKEVHSGVSTDPQPLKEQPALNDSRYCLSSRLRVSATFWQNPRNHERCQV |
| | | QFYGLSENDEWTQDRAKPVTQIVSAEAWGRADCGFTSESYQQGVLSATIL |
| | | YEILLGKATLYAVLVSALVLMAMVKRKDSRG |
| CD21 | 8 | MGAAGLLGVFLALVAPGVLGISCGSPPPILNGRISYYSTPIAVGTVI |
| | | RYSCSGTFRLIGEKSLLCITKDKVDGTWDKPAPKCEYENKYSSCPEPIVP |
| | | GGYKIRGSTPYRHGDSVTFACKTNFSMNGNKSVWCQANNMWGPTRLPTCV |
| | | SVFPLECPALPMIHNGHHTSENVGSIAPGLSVTYSCESGYLLVGEKIINC |
| | | LSSGKWSAVPPTCEEARCKSLGREPNGKVKEPPILRVGVTANFFCDEGYR |
| | | LQGPPSSRCVIAGQGVAWTKMPVCEEIFCPSPPPILNGRHIGNSLANVSY |
| | | GSIVTYTCDPDPEEGVNFILIGESTLRCTVDSQKTGTWSGPAPRCELSTS |
| | | AVQCPHPQILRGRMVSGQKDRYTYNDTVIFACMFGFTLKGSKQIRCNAQG |
| | | TWEPSAPVCEKECQAPPNILNGQKEDRHMVREDPGTSIKYSCNPGYVLVG |
| | | EESIQCTSEGVWTPPVPQCKVAACEATGRQLLTKPQHQFVRPDVNSSCGE |
| | | GYKLSGSVYQECQGTIPWEMEIRLCKEITCPPPPVIYNGAHTGSSLEDEP |
| | | YGTTVTYTCNPGPERGVEFSLIGESTIRCTSNDQERGTWSGPAPLCKLSL |
| | | LAVQCSHVHIANGYKISGKEAPYFYNDTVTFKCYSGFTLKGSSQIRCKAD |
| | | NTWDPEIPVCEKETCQHVRQSLQELPAGSRVELVNTSCQDGYQLTGHAYQ |
| | | MCQDAENGIWFKKIPLCKVIHCHPPPVIVNGKHTGMMAENFLYGNEVSYE |
| | | CDQGFYLLGEKKLQCRSDSKGHGSWSGPSPQCLRSPPVTRCPNPEVKHGY |
| | | KLNKTHSAYSHNDIVYVDCNPGFIMNGSRVIRCHTDNTWVPGVPTCIKKA |
| | | FIGCPPPPKTPNGNHTGGNIARFSPGMSILYSCDQGYLLVGEALLLCTHE |
| | | GTWSQPAPHCKEVNCSSPADMDGIQKGLEPRKMYQYGAVVTLECEDGYML |
| | | EGSPQSQCQSDHQWNPPLAVCRSRSLAPVLCGIAAGLILLTFLIVITLYV |
| | | ISKHRARNYYTDTSQKEAFHLEAREVYSVDPYNPAS |
| CCR9 | 9 | MTPTDFTSPIPNMADDYGSESTSSMEDYVNFNFTDFYCEKNNVRQFA |
| | | SHELPPLYWLVFIVGALGNSLVILVYWYCTRVKTMTDMELLNLAIADLLF |
| | | LVTLPFWAIAAADQWKFQTFMCKVVNSMYKMNFYSCVLLIMCISVDRYIA |
| | | IAQAMRAHTWREKRLLYSKMVCFTIWVLAAALCIPEILYSQIKEESGIAI |
| | | CTMVYPSDESTKLKSAVLTLKVILGFFLPFVVMACCYTIIIHTLIQAKKS |
| | | SKHKALKVTITVLTVFVLSQFPYNCILLVQTIDAYAMFISNCAVSTNIDI |
| | | CFQVTQTIAFFHSCLNPVLYVFVGERFRRDLVKTLKNLGCISQAQWVSFT |
| | | RREGSLKLSSMLLETTSGALSL |
| CD30 | 10 | MRVLLAALGLLELGALRAFPQDRPFEDTCHGNPSHYYDKAVRRCCYR |
| | | CPMGLFPTQQCPQRPTDCRKQCEPDYYLDEADRCTACVTCSRDDLVEKTP |
| | | CAWNSSRVCECRPGMFCSTSAVNSCARCFFHSVCPAGMIVKFPGTAQKNT |
| | | VCEPASPGVSPACASPENCKEPSSGTIPQAKPTPVSPATSSASTMPVRGG |
| | | TRLAQEAASKLTRAPDSPSSVGRPSSDPGLSPTQPCPEGSGDCRKQCEPD |
| | | YYLDEAGRCTACVSCSRDDLVEKTPCAWNSSRTCECRPGMICATSATNSC |
| | | ARCVPYPICAAETVTKPQDMAEKDTTFEAPPLGTQPDCNPTPENGEAPAS |
| | | TSPTQSLLVDSQASKTLPIPTSAPVALSSTGKPVLDAGPVLEWVILVLVV |
| | | VVGSSAFLLCHRRACRKRIRQKLHLCYPVQTSQPKLELVDSRPRRSSTQL |
| | | RSGASVTEPVAEERGLMSQPLMETCHSVGAAYLESLPLQDASPAGGPSSP |
| | | RDLPEPRVSTEHTNNKIEKIYIMKADTVIVGTVKAELPEGRGLAGPAEPE |
| | | LEEELEADHTPHYPEQETEPPLGSCSDVMLSVEEEGKEDPLPTAASGK |
| CD123 | 11 | MVLLWLTLLLIALPCLLQTKEDPNPPITNLRMKAKAQQLTWDLNRNV |
| | | TDIECVKDADYSMPAVNNSYCQFGAISLCEVTNYTVRVANPPESTWILFP |
| | | ENSGKPWAGAENLTCWIHDVDFLSCSWAVGPGAPADVQYDLYLNVANRRQ |
| | | QYECLHYKTDAQGTRIGCREDDISRLSSGSQSSHILVRGRSAAFGIPCTD |
| | | KFVVFSQIEILTPPNMTAKCNKTHSFMHWKMRSHENRKFRYELQIQKRMQ |
| | | PVITEQVRDRTSFQLLNPGTYTVQIRARERVYEFLSAWSTPQRFECDQEE |
| | | GANTRAWRTSLLIALGTLLALVCVFVICRRYLVMQRLFPRIPHMKDPIGD |
| | | SFQNDKLVVWEAGKAGLEECLVTEVQVVQKT |
| CD33 | 12 | MPLLLLLPLLWAGALAMDPNFWLQVQESVTVQEGLCVLVPCTFFHPI |
| | | PYYDKNSPVHGYWFREGAIISRDSPVATNKLDQEVQEETQGRERLLGDPS |
| | | RNNCSLSIVDARRRDNGSYFERMERGSTKYSYKSPQLSVHVTDLTHRPKI |
| | | LIPGTLEPGHSKNLTCSVSWACEQGTPPIESWLSAAPTSLGPRTTHSSVL |
| | | IITPRPQDHGTNLTCQVKFAGAGVTTERTIQLNVTYVPQNPTTGIFPGDG |
| | | SGKQETRAGVVHGAIGGAGVTALLALCLCLIFFIVKTHRRKAARTAVGRN |
| | | DTHPTTGSASPKHQKKSKLHGPTETSSCSGAAPTVEMDEELHYASLNFHG |
| | | MNPSKDTSTEYSEVRTQ |
| CD38 | 13 | MANCEFSPVSGDKPCCRLSRRAQLCLGVSILVLILVVVLAVVVPRWR |
| | | QQWSGPGTTKRFPETVLARCVKYTEIHPEMRHVDCQSVWDAFKGAFISKH |
| | | PCNITEEDYQPLMKLGTQTVPCNKILLWSRIKDLAHQFTQVQRDMFTLED |
| | | TLLGYLADDLTWCGEENTSKINYQSCPDWRKDCSNNPVSVFWKTVSRRFA |
| | | EAACDVVHVMLNGSRSKIFDKNSTFGSVEVHNLQPEKVQTLEAWVIHGGR |
| | | EDSRDLCQDPTIKELESIISKRNIQFSCKNIYRPDKFLQCVKNPEDSSCT |
| | | SEI |
| CD138 | 14 | MRRAALWLWLCALALSLQPALPQIVATNLPPEDQDGSGDDSDNESGS |
| | | GAGALQDITLSQQTPSTWKDTQLLTAIPTSPEPTGLEATAASTSTLPAGE |
| | | GPKEGEAVVLPEVEPGLTAREQEATPRPRETTQLPTTHLASTTTATTAQE |
| | | PATSHPHRDMQPGHHETSTPAGPSQADLHTPHTEDGGPSATERAAEDGAS |
| | | SQLPAAEGSGEQDFTFETSGENTAVVAVEPDRRNQSPVDQGATGASQGLL |
| | | DRKEVLGGVIAGGLVGLIFAVCLVGFMLYRMKKKDEGSYSLEEPKQANGG |
| | | AYQKPTKQEEFYA |
| CLL-1 | 15 | MSEEVTYADLQFQNSSEMEKIPEIGKFGEKAPPAPSHVWRPAALELT |
| | | LLCLLLLIGLGVLASMFHVTLKIEMKKMNKLQNISEELQRNISLQLMSNM |
| | | NISNKIRNLSTTLQTIATKLCRELYSKEQEHKCKPCPRRWIWHKDSCYEL |
| | | SDDVQTWQESKMACAAQNASLLKINNKNALEFIKSQSRSYDYWLGLSPEE |
| | | DSTRGMRVDNIINSSAWVIRNAPDLNNMYCGYINRLYVQYYHCTYKKRMI |
| | | CEKMANPVQLGSTYFREA |
| LILRB4 | 16 | MIPTFTALLCLGLSLGPRTHMQAGPLPKPTLWAEPGSVISWGNSVTI |
| | | WCQGTLEAREYRLDKEESPAPWDRQNPLEPKNKARFSIPSMTEDYAGRYR |
| | | CYYRSPVGWSQPSDPLELVMTGAYSKPTLSALPSPLVTSGKSVTLLCQSR |
| | | SPMDTFLLIKERAAHPLLHLRSEHGAQQHQAEFPMSPVTSVHGGTYRCES |
| | | SHGFSHYLLSHPSDPLELIVSGSLEDPRPSPTRSVSTAAGPEDQPLMPTG |
| | | SVPHSGLRRHWEVLIGVLVVSILLLSLLLFLLLQHWRQGKHRTLAQRQAD |
| | | FQRPPGAAEPEPKDGGLQRRSSPAADVQGENFCAAVKNTQPEDGVEMDTR |
| | | QSPHDEDPQAVTYAKVKHSRPRREMASPPSPLSGEFLDTKDRQAEEDRQM |
| | | DTEAAASEAPQDVTYAQLHSFTLRQKATEPPPSQEGASPAEPSVYATLAI |
| | | H |
| Siglec-6 | 17 | MQGAQEASASEMLPLLLPLLWAGALAQERRFQLEGPESLTVQEGLCV |
| | | LVPCRLPTTLPASYYGYGYWFLEGADVPVATNDPDEEVQEETRGREHLLW |
| | | DPRRKNCSLSIRDARRRDNAAYFFRLKSKWMKYGYTSSKLSVRVMALTHR |
| | | PNISIPGTLESGHPSNLTCSVPWVCEQGTPPIFSWMSAAPTSLGPRTTQS |
| | | SVLTITPRPQDHSTNLTCQVTFPGAGVTMERTIQLNVSYAPQKVAISIFQ |
| | | GNSAAFKILQNTSSLPVLEGQALRLLCDADGNPPAHLSWFQGFPALNATP |
| | | ISNTGVLELPQVGSAEEGDFTCRAQHPLGSLQISLSLFVHWKPEGRAGGV |
| | | LGAVWGASITTLVFLCVCFIFRVKTRRKKAAQPVQNTDDVNPVMVSGSRG |
| | | HQHQFQTGIVSDHPAEAGPISEDEQELHYAVLHFHKVQPQEPKVTDTEYS |
| | | EIKIHK |
| CD70 | 18 | MPEEGSGCSVRRRPYGCVLRAALVPLVAGLVICLVVCIQRFAQAQQQ |
| | | LPLESLGWDVAELQLNHTGPQQDPRLYWQGGPALGRSFLHGPELDKGQLR |
| | | IHRDGIYMVHIQVTLAICSSTTASRHHPTTLAVGICSPASRSISLLRLSE |
| | | HQGCTIASQRLTPLARGDTLCTNLTGTLLPSRNTDETFFGVQWVRP |
| PD-L1 | 19 | MRIFAVFIFMTYWHLLNAFTVTVPKDLYVVEYGSNMTIECKFPVEKQ |
| | | LDLAALIVYWEMEDKNIIQFVHGEEDLKVQHSSYRQRARLLKDQLSLGNA |
| | | ALQITDVKLQDAGVYRCMISYGGADYKRITVKVNAPYNKINQRILVVDPV |
| | | TSEHELTCQAEGYPKAEVIWTSSDHQVLSGKTTTTNSKREEKLENVTSTL |
| | | RINTTTNEIFYCTFRRLDPEENHTAELVIPELPLAHPPNERTHLVILGAI |
| | | LLCLGVALTFIFRLRKGRMMDVKKCGIQDTNSKKQSDTHLEET |
| CD19 | 20 | MPPPRLLFFLLELTPMEVRPEEPLVVKVEEGDNAVLQCLKGTSDGPT |
| | | QQLTWSRESPLKPFLKLSLGLPGLGIHMRPLAIWLFIFNVSQQMGGFYLC |
| | | QPGPPSEKAWQPGWTVNVEGSGELFRWNVSDLGGLGCGLKNRSSEGPSSP |
| | | SGKLMSPKLYVWAKDRPEIWEGEPPCLPPRDSLNQSLSQDLTMAPGSTLW |
| | | LSCGVPPDSVSRGPLSWTHVHPKGPKSLLSLELKDDRPARDMWVMETGLL |
| | | LPRATAQDAGKYYCHRGNLTMSFHLEITARPVLWHWLLRTGGWKVSAVTL |
| | | AYLIFCLCSLVGILHLQRALVLRRKRKRMTDPTRRFFKVTPPPGSGPQNQ |
| | | YGNVLSLPTPTSGLGRAQRWAAGLGGTAPSYGNPSSDVQADGALGSRSPP |
| | | GVGPEEEEGEGYEEPDSEEDSEFYENDSNLGQDQLSQDGSGYENPEDEPL |
| | | GPEDEDSESNAESYENEDEELTQPVARTMDELSPHGSAWDPSREATSLGS |
| | | QSYEDMRGILYAAPQLRSIRGQPGPNHEEDADSYENMDNPDGPDPAWGGG |
| | | GRMGTWSTR |
| ROR1 | 21 | MHRPRRRGTRPPLLALLAALLLAARGAAAQETELSVSAELVPTSSWN |
| | | ISSELNKDSYLTLDEPMNNITTSLGQTAELHCKVSGNPPPTIRWEKNDAP |
| | | VVQEPRRLSFRSTIYGSRLRIRNLDTTDTGYFQCVATNGKEVVSSTGVLE |
| | | VKFGPPPTASPGYSDEYEEDGFCQPYRGIACARFIGNRTVYMESLHMQGE |
| | | IENQITAAFTMIGTSSHLSDKCSQFAIPSLCHYAFPYCDETSSVPKPRDL |
| | | CRDECEILENVLCQTEYIFARSNPMILMRLKLPNCEDLPQPESPEAANCI |
| | | RIGIPMADPINKNHKCYNSTGVDYRGTVSVTKSGRQCQPWNSQYPHTHTF |
| | | TALRFPELNGGHSYCRNPGNQKEAPWCFTLDENFKSDLCDIPACDSKDSK |
| | | EKNKMEILYILVPSVAIPLAIALLFFFICVCRNNQKSSSAPVQRQPKHVR |
| | | GQNVEMSMLNAYKPKSKAKELPLSAVREMEELGECAFGKIYKGHLYLPGM |
| | | DHAQLVAIKTLKDYNNPQQWTEFQQEASLMAELHHPNIVCLLGAVTQEQP |
| | | VCMLFEYINQGDLHEFLIMRSPHSDVGCSSDEDGTVKSSLDHGDELHIAI |
| | | QIAAGMEYLSSHFFVHKDLAARNILIGEQLHVKISDLGLSREIYSADYYR |
| | | VQSKSLLPIRWMPPEAIMYGKFSSDSDIWSFGVVLWEIFSFGLQPYYGES |
| | | NQEVIEMVRKRQLLPCSEDCPPRMYSLMTECWNEIPSRRPREKDIHVRLR |
| | | SWEGLSSHTSSTTPSGGNATTQTTSLSASPVSNLSNPRYPNYMFPSQGIT |
| | | PQGQIAGFIGPPIPQNQRFIPINGYPIPPGYAAFPAAHYQPTGPPRVIQH |
| | | CPPPKSRSPSSASGSTSTGHVTSLPSSGSNQEANIPLLPHMSIPNHPGGM |
| | | GITVFGNKSQKPYKIDSKQASLLGDANIHGHTESMISAEL |
| WT1 | 22 | MGSDVRDLNALLPAVPSLGGGGGCALPVSGAAQWAPVLDEAPPGASA |
| | | YGSLGGPAPPPAPPPPPPPPPHSFIKQEPSWGGAEPHEEQCLSAFTVHES |
| | | GQFTGTAGACRYGPFGPPPPSQASSGQARMEPNAPYLPSCLESQPAIRNQ |
| | | GYSTVTFDGTPSYGHTPSHHAAQFPNHSFKHEDPMGQQGSLGEQQYSVPP |
| | | PVYGCHTPTDSCTGSQALLLRTPYSSDNLYQMTSQLECMTWNQMNLGATL |
| | | KGVAAGSSSSVKWTEGQSNHSTGYESDNHTTPILCGAQYRIHTHGVERGI |
| | | QDVRRVPGVAPTLVRSASETSEKRPFMCAYPGCNKRYFKLSHLQMHSRKH |
| | | TGEKPYQCDFKDCERRESRSDQLKRHQRRHTGVKPFQCKTCQRKFSRSDH |
| | | LKTHTRTHTGKTSEKPFSCRWPSCQKKFARSDELVRHHNMHQRNMTKLQL |
| | | AL |
| PRAME | 23 | MERRRLWGSIQSRYISMSVWTSPRRLVELAGQSLLKDEALAIAALEL |
| | | LPRELFPPLEMAAFDGRHSQTLKAMVQAWPFTCLPLGVLMKGQHLHLETE |
| | | KAVLDGLDVLLAQEVRPRRWKLQVLDLRKNSHQDFWTVWSGNRASLYSFP |
| | | EPEAAQPMTKKRKVDGLSTEAEQPFIPVEVLVDLELKEGACDELFSYLIE |
| | | KVKRKKNVLRLCCKKLKIFAMPMQDIKMILKMVQLDSIEDLEVTCTWKLP |
| | | TLAKFSPYLGQMINLRRLLLSHIHASSYISPEKEEQYIAQFTSQFLSLQC |
| | | LQALYVDSLFFLRGRLDQLLRHVMNPLETLSITNCRLSEGDVMHLSQSPS |
| | | VSQLSVLSLSGVMLTDVSPEPLQALLERASATLQDLVEDECGITDDQLLA |
| | | LLPSLSHCSQLTTLSFYGNSISISALQSLLQHLIGLSNLTHVLYPVPLES |
| | | YEDIHGTLHLERLAYLHARLRELLCELGRPSMVWLSANPCPHCGDRTFYD |
| | | PEPILCPCEMPN |
| HA-1 | 24 | MFSRKKRELMKTPSISKKNRAGSPSPQPSGELPRKDGADAVFPGPSL |
| | | EPPAGSSGVKATGTLKRPTSLSRHASAAGFPLSGAASWTLGRSHRSPLTA |
| | | ASPGELPTEGAGPDVVEDISHLLADVARFAEGLEKLKECVLRDDLLEARR |
| | | PRAHECLGEALRVMHQIISKYPLLNTVETLTAAGTLIAKVKAFHYESNND |
| | | LEKQEFEKALETIAVAFSSTVSEFLMGEVDSSTLLAVPPGDSSQSMESLY |
| | | GPGSEGTPPSLEDCDAGCLPAEEVDVLLQRCEGGVDAALLYAKNMAKYMK |
| | | DLISYLEKRTTLEMEFAKGLQKIAHNCRQSVMQEPHMPLLSIYSLALEQD |
| | | LEFGHSMVQAVGTLQTQTFMQPLTLRRLEHEKRRKEIKEAWHRAQRKLQE |
| | | AESNLRKAKQGYVQRCEDHDKARELVAKAEEEQAGSAPGAGSTATKTLDK |
| | | RRRLEEEAKNKAEEAMATYRTCVADAKTQKQELEDTKVTALRQIQEVIRQ |
| | | SDQTIKSATISYYQMMHMQTAPLPVHFQMLCESSKLYDPGQQYASHVRQL |
| | | QRDQEPDVHYDFEPHVSANAWSPVMRARKSSENVSDVARPEAAGSPPEEG |
| | | GCTEGTPAKDHRAGRGHQVHKSWPLSISDSDSGLDPGPGAGDEKKFERTS |
| | | SSGTMSSTEELVDPDGGAGASAFEQADLNGMTPELPVAVPSGPFRHEGLS |
| | | KAARTHRLRKLRTPAKCRECNSYVYFQGAECEECCLACHKKCLETLAIQC |
| | | GHKKLQGRLQLFGQDESHAARSAPDGVPFIVKKCVCEIERRALRTKGIYR |
| | | VNGVKTRVEKLCQAFENGKELVELSQASPHDISNVLKLYLRQLPEPLISF |
| | | RLYHELVGLAKDSLKAEAEAKAASRGRQDGSESEAVAVALAGRLRELLRD |
| | | LPPENRASLQYLLRHLRRIVEVEQDNKMTPGNLGIVEGPTLLRPRPTEAT |
| | | VSLSSLVDYPHQARVIETLIVHYGLVFEEEPEETPGGQDESSNQRAEVVV |
| | | QVPYLEAGEAVVYPLQEAAADGCRESRVVSNDSDSDLEEASELLSSSEAS |
| | | ALGHLSFLEQQQSEASLEVASGSHSGSEEQLEATAREDGDGDEDGPAQQL |
| | | SGFNTNQSNNVLQAPLPPMRLRGGRMTLGSCRERQPEFV |
| NPM1 | 25 | MEDSMDMDMSPLRPQNYLFGCELKADKDYHFKVDNDENEHQLSLRTV |
| | | SLGAGAKDELHIVEAEAMNYEGSPIKVTLATLKMSVQPTVSLGGFEITPP |
| | | VVLRLKCGSGPVHISGQHLVAVEEDAESEDEEEEDVKLLSISGKRSAPGG |
| | | GSKVPQKKVKLAADEDDDDDDEEDDDEDDDDDDEDDEEAEEKAPVKKSIR |
| | | DTPAKNAQKSNQNGKDSKPSSTPRSKGQESFKKQEKTPKTPKGPSSVEDI |
| | | KAKMQASIEKGGSLPKVEAKFINYVKNCFRMTDQEAIQDLWQWRKSL |
| — | 26 | RMFPNAPYL |
| — | 27 | CMTWNQMNL |
| — | 28 | VLDGLDVLL |
| — | 29 | ALYVDSLEFL |
| — | 30 | SLYSFPEPEA |
| — | 31 | SLLQHLIGL |
| — | 32 | LYVDSLFFLC |
| — | 33 | VLHDDLLEA |
| — | 34 | CLAVEEVSL |
| — | 35 | KRYFKLSHLQMHSRKH |
| Anti-CD7 | 36 | VL: |
| scFV | | GAQPAMAAYKDIQMTQTTSSLSASLGDRVTISCSASQGISNYLNWYQQKP |
| | | DGTVKLLIYYTSSLHSGVPSRFSGSGSGTDYSLTISNLEPEDIATYYCQQ |
| | | YSKLPYTFGGGTKLEIKR |
| | | VH: |
| | | EVQLVESGGGLVKPGGSLKLSCAASGLTESSYAMSWVRQTPEKRLEWVAS |
| | | ISSGGFTYYPDSVKGRFTISRDNARNILYLQMSSLRSEDTAMYYCARDEV |
| | | RGYLDVWGAGTTVTVSSASGA |
| Anti-CD19 | 37 | VL: |
| scFV | | DIQMTQTTSSLSASLGDRVTISCRASQDISKYLNWYQQKPDGTVKLLIYH |
| | | TSRLHSGVPSRESGSGSGTDYSLTISNLEQEDIATYFCQQGNTLPYTFGG |
| | | GTKLEIT |
| | | VH: |
| | | EVKLQESGPGLVAPSQSLSVTCTVSGVSLPDYGVSWIRQPPRKGLEWLGV |
| | | IWGSETTYYNSALKSRLTIIKDNSKSQVFLKMNSLQTDDTAIYYCAKHYY |
| | | YGGSYAMDYWGQGTSVTVSS |
| Anti-ROR1 | 38 | VL: |
| scFV | | DIVMTQSQKIMSTTVGDRVSITCKASQNVDAAVAWYQQKPGQSPKLLIYS |
| | | ASNRYTGVPDRFTGSGSGTDFTLTISNMQSEDLADYFCQQYDIYPYTFGG |
| | | GTKLEIK |
| | | VH: |
| | | QVQLQQSGAELVRPGASVTLSCKASGYTFSDYEMHWVIQTPVHGLEWIGA |
| | | IDPETGGTAYNQKFKGKAILTADKSSSTAYMELRSLTSEDSAVYYCTGYY |
| | | DYDSFTYWGQGTLVTVSA |

### Therapeutic Methods, Uses, and Compositions

The CD7 CAR-modified INKT cells (e.g., CD3⁺iTCR(Vα24-Jα18)⁺, or CD3⁺Vα24⁺ INKT cells, also known as type I NKT cells) described herein can be employed in various therapeutic and prophylactic applications. For instance, in some embodiments, the CD7 CAR-modified iNKT cells described herein may be useful in treating or preventing a CD7⁺ cancer. The CD7 CAR-modified INKT cells described herein may be administered per se or in any suitable pharmaceutical composition.

Accordingly, in certain aspects, the present disclosure provides methods of treating or preventing a CD7⁺ cancer in a subject in need thereof by administering to the subject a therapeutically effective amount of CD7 CAR-modified INKT cells (e.g., CD3⁺iTCR(Vα24-Jα18)⁺, or CD3⁺Vα24⁺ INKT cells). In certain aspects, the present disclosure further provides methods of preparing a therapy for treating or preventing a CD7⁺ cancer in a subject in need thereof by: (a) isolating one or more INKT cells (e.g., CD3⁺iTCR(Vα24-Jα18)⁺ or CD3⁺Vα24⁺ INKT cells) from a biological sample; (b) enabling or activating the one or more iNKT cells in a growth medium for proliferation using but not limited to irradiated autologous PBMC negative fraction and/or whole PBMCs, α-galactosylceramide (αGalCer) or 7DW8-5, IL-2, IL-15 and/or IL-7; (c) engineering activating or proliferating iNKT cells to express CAR (e.g., CD7) using a lentiviral vector; and (d) expanding the one or more CAR (e.g., CD7) engineered INKT cells in a growth medium using our own protocol of irradiated allogeneic PBMCs and EBV-B cells, αGalCer or 7DW8-5, and mixed cytokines (IL-2, IL-15 and/or IL-7). In certain aspects, the present disclosure further provides methods of treating or preventing a CD7⁺ cancer in a subject in need thereof by administering to the subject a therapeutically effective amount of an expanded cell population (e.g., an expanded cell population comprising CD7 CAR-modified INKT cells (e.g., CD3⁺iTCR(Vα24-Jα18)⁺ or CD3⁺Vα24⁺ INKT cells), as described herein and/or prepared by a method described herein). Uses of the disclosed CD7 CAR-modified INKT cells, e.g., in treating or preventing a CD7⁺ cancer, are also provided. Pharmaceutical compositions comprising a therapeutically effective amount of CD7 CAR-modified INKT cells (e.g., CD3⁺iTCR(Vα24-Jα18)⁺ or CD3⁺Vα24⁺ INKT cells) are also disclosed, and are useful in the therapeutic methods and uses provided herein.

An exemplary embodiment is a method of treating or preventing a CD7⁺ cancer in a subject in need thereof, comprising administering to the subject a therapeutically effective amount of CD7 CAR-modified INKT cells (e.g., CD3⁺iTCR(Vα24-Jα18)⁺ or CD3⁺Vα24⁺ INKT cells), or a pharmaceutical composition comprising a therapeutically effective amount of CD7 CAR-modified INKT cells (e.g., CD3⁺iTCR(Vα24-Jα18)⁺ or CD3⁺Vα24⁺ INKT cells) and at least one pharmaceutically acceptable carrier.

Another exemplary embodiment is a method of preparing a therapy for treating or preventing a CD7⁺ cancer in a subject in need thereof, comprising (a) isolating one or more INKT cells (e.g., CD3⁺iTCR(Vα24-Jα18)⁺ or CD3⁺Vα24⁺ INKT cells) from a biological sample; (b) enabling or activating the one or more iTCR⁺ INKT cells in a growth medium for proliferation using but not limited to irradiated autologous PBMC negative fraction and/or whole PBMC, α-galactosylceramide (αGalCer) or 7DW8-5, IL-2, IL-15 and/or IL-7; (c) engineering activating or proliferating iNKT cells to express CAR (e.g., CD7) using a lentiviral vector; and (d) expanding the one or more CAR (e.g., CD7) engineered iNK T cells in a growth medium using our own protocol of irradiated allogeneic PBMCs and EBV-B cells, αGalCer or 7DW8-5, and mixed cytokines (IL-2, IL-15 and/or IL-7).

Another exemplary embodiment is a method of treating or preventing a CD7⁺ cancer in a subject in need thereof, comprising administering to the subject a therapeutically effective amount of an expanded cell population (e.g., an expanded cell population comprising CD7 CAR-modified INKT cells (e.g., CD3⁺iTCR(Vα24-Jα18)⁺ or CD3⁺Vα24⁺ INKT cells), as described herein and/or prepared by a method described herein), or a pharmaceutical composition comprising a therapeutically effective amount of an expanded cell population (e.g., an expanded cell population comprising CD7 CAR-modified INKT cells (e.g., CD3⁺iTCR(Vα24-Jα18)⁺ or CD3⁺Vα24⁺ INKT cells), as described herein and/or prepared by a method described herein) and at least one pharmaceutically acceptable carrier.

Another exemplary embodiment is isolated INKT cells (e.g., CD3⁺iTCR(Vα24-Jα18)⁺ or CD3⁺Vα24⁺ INKT cells) and modifying to express CD7 CAR for use in treating or preventing a CD7⁺ cancer in a subject in need thereof. In some embodiments, the use comprises administering to the subject a therapeutically effective amount of the cells, or a pharmaceutical composition comprising a therapeutically effective amount of the cells and at least one pharmaceutically acceptable carrier.

Another exemplary embodiment is use of isolated INKT cells (e.g., CD3⁺iTCR(Vα24-Jα18)⁺ or CD3⁺Vα24⁺ INKT cells) in treating or preventing a CD7⁺ cancer in a subject in need thereof. In some embodiments, the use comprises administering to the subject a therapeutically effective amount of the cells, or a pharmaceutical composition comprising a therapeutically effective amount of the cells and at least one pharmaceutically acceptable carrier.

Another exemplary embodiment is use of isolated INKT cells (e.g., CD3⁺iTCR(Vα24-Jα18)⁺ or CD3⁺Vα24⁺ INKT cells) in the manufacture of a medicament for treating or preventing a CD7⁺ cancer in a subject in need thereof. In some embodiments, the medicament comprises a therapeutically effective amount of the cells, or a pharmaceutical composition comprising a therapeutically effective amount of the cells and at least one pharmaceutically acceptable carrier.

As used herein, the term “treat” and its cognates refer to an amelioration of a disease, disorder, or condition (e.g., a cancer), or at least one discernible symptom thereof. The term “treat” encompasses but is not limited to complete treatment or complete amelioration of one or more symptoms of a cancer. In some embodiments, “treat” refers to at least partial amelioration of at least one measurable physical parameter, not necessarily discernible by the subject. In some embodiments, “treat” refers to inhibiting the progression of a disease, disorder, or condition, either physically (e.g., stabilization of a discernible symptom), physiologically (e.g., stabilization of a physical parameter), or both. In some embodiments, “treat” refers to slowing the progression or reversing the progression of a disease, disorder, or condition. As used herein, “treat” and its cognates also encompass delaying the onset or reducing the risk of acquiring a given disease, disorder, or condition. In some embodiments, “treat” refers to administering to a subject suspected of having a disease, disorder, or condition (e.g., a cancer or a precancerous condition) a CD7 CAR-modified iNKT cell, cell population, or composition disclosed herein. In some embodiments, a subject and/or a sample from a subject suspected of having a CD7⁺ cancer and/or a precancerous condition may comprise one or more cells that are abnormal, malignant, and/or premalignant.

The terms “subject” and “patient” are used interchangeably herein to refer to any human or non-human animal in need of treatment. Non-human animals include all vertebrates (e.g., mammals and non-mammals). Non-limiting examples of mammals include humans, mice, rats, rabbits, dogs, monkeys, and pigs. In some embodiments, the subject is a human.

The term “donor,” as used herein, refers to any human or non-human animal that donates a biological sample (e.g., a blood sample) for use in a subject in need of treatment and/or for use in preparing a therapy (e.g., a CD7 CAR-modified iNKT cell therapy disclosed herein) for a subject in need of treatment. In some embodiments, the donor is a huma.

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Cancer cells can be in the form of a tumor or mass, but such cells may exist alone within a subject, or may circulate in the blood stream as independent cells, such as leukemia or lymphoma cells. The term “cancer” includes all types of cancers and cancer metastases, including hematological malignancies, solid tumors, sarcomas, carcinomas, and other solid and non-solid tumor cancers.

The term “CD7⁺ cancer”, as used herein, refers to cancer cells expressing CD7 antigen on the cell surface and includes, but not limited to, T-cell lymphoblastic leukemias (T-ALL) and T-ALL subtypes including early thymic precursors (ETP)-ALL (ETP-ALL), Pro-T-ALL, Pre-T-ALL, Cortical T-ALL and Mature T-ALL; a subset of peripheral T-cell lymphomas (PTCLs) and PTCL subtypes including PTCL, not otherwise specified (PTCL-NOS), anaplastic large cell lymphoma (ALCL), primary cutaneous ALCL, angioimmunoblastic T-cell lymphoma (AITL), nasal NK/T-cell lymphoma, adult T-cell acute lymphoblastic lymphoma or leukemia (ATLL) associated with the human T-cell leukemia virus-1 (HTLV-1) infection, enteropathy-associated lymphoma, hepatosplenic lymphoma, subcutaneous panniculitis-like lymphoma, precursor T-cell acute lymphoblastic lymphoma or leukemia, blastic NK-cell lymphoma, and cutaneous T-cell lymphomas (CTCLs); CD7⁺ acute myeloid leukemia (AML); CD7⁺ other malignancies or CD7⁺CD1d⁺ malignancies. In some embodiments, a CD7⁺ cancer is T-ALL or AML. In some embodiments, a CD7⁺ cancer is a refractory or relapsed cancer (e.g., refractory or relapsed T-ALL or AML). In some embodiments, a cancer is refractory or relapsed T-ALL or AML. In some embodiments, a cancer expresses a target antigen.

The term “target antigen,” as used herein, refers to any antigen targeted by a CD7 CAR-modified INKT cell and/or by a construct expressed by an INKT cell (e.g., a CAR, TCR, or TCRm). As used herein, the term “antigen” is synonymous with “antigenic determinant” and “epitope,” and refers to a site (e.g., a contiguous stretch of amino acids or a conformational configuration made up of different regions of non-contiguous amino acids) on a polypeptide macromolecule to which an antigen binding moiety (e.g., an antigen binding moiety of a CAR, TCR, or TCRm) binds, forming an antigen binding moiety-antigen complex. Useful antigenic determinants can be found, for example, within or on the surfaces of cancer cells, within or on the surfaces of virus-infected cells, within or on the surfaces of other diseased cells, free in blood serum, and/or in the extracellular matrix (ECM).

Exemplary target antigens are disclosed herein, and include without limitation CD7, CD1a, CD1d, CD2, CD5, TRBC1, CD21, CCR9, CD30, CD123, CD33, CD38, CD138, CLL-1, LILRB4, Siglec-6, CD70, PD-L1, CD19, ROR1, WT1, PRAME, HA-1, and ΔNPM1. A target antigen may include a full-length antigen (e.g., any of the exemplary antigens disclosed herein), as well as any form of the antigen that may result from cellular processing. A target antigen also encompasses functional variants or fragments of an antigen (e.g., any of the exemplary antigens disclosed herein), including but not limited to splice variants, allelic variants, and isoforms that retain one or more biologic functions of the antigen (i.e., variants and fragments are encompassed unless the context indicates that the term is used to refer to the wild-type antigen only). In some embodiments, a target antigen is a functional fragment of a full-length antigen.

In some embodiments of the methods and uses described herein, the CD7 CAR-modified INKT cells are formulated and/or used as a pharmaceutical composition. Accordingly, in certain aspects, the present disclosure provides pharmaceutical compositions comprising CD7 CAR-modified INKT cells. An exemplary embodiment is a pharmaceutical composition, e.g., for treating or preventing a CD7⁺ cancer in a subject in need thereof, comprising CD7 CAR-modified INKT cells (e.g., CD3⁺iTCR(Vα24-Jα18)⁺ or CD3⁺Vα24⁺ INKT cells). In some embodiments, a pharmaceutical composition further comprises at least one pharmaceutically acceptable carrier. Pharmaceutical compositions may also comprise one or more additional therapeutic agents that are suitable for treating or preventing, for example, a cancer (e.g., an anti-cancer agent, a standard-of-care agent for the particular cancer being treated, etc.). Pharmaceutical compositions may also comprise one or more inactive carriers, excipients, and/or stabilizer components, and the like. Methods of formulating pharmaceutical compositions and suitable formulations (e.g., for intravenous, systemic, or other modes of administration) are known in the art (see, e.g., “Remington's Pharmaceutical Sciences,” Mack Publishing Co., Easton, PA). Appropriate formulation may depend on the route of administration.

As used herein, a “pharmaceutical composition” refers to a preparation of an NKT cell or cell population (e.g., a CD3⁺iTCR(Vα24-Jα18)⁺ or CD3⁺Vα24⁺ INKT cell or cell population) and, optionally, comprising one or more additional components suitable for administration to a subject, such as a physiologically acceptable carrier and/or excipient. The pharmaceutical compositions provided herein are in such form as to permit administration and subsequently provide the intended biological activity of the active ingredient(s) and/or to achieve a therapeutic effect. The pharmaceutical compositions provided herein contain no additional components which are unacceptably toxic to a subject to which the formulation would be administered.

As used herein, the phrases “pharmaceutically acceptable carrier” and “physiologically acceptable carrier,” which may be used interchangeably, refer to a carrier or a diluent that does not cause significant irritation to a subject and does not abrogate the biological activity and properties of the administered INKT cell or cell population or any additional therapeutic agent in the composition. Pharmaceutically acceptable carriers may enhance or stabilize the composition and/or can be used to facilitate preparation of the composition. Pharmaceutically acceptable carriers can include solvents, dispersion media, coatings, antibacterial and antifungal agents, isotonic and absorption delaying agents, and the like that are physiologically compatible. The carrier may be selected to minimize adverse side effects in the subject, and/or to minimize degradation of the active ingredient(s). An adjuvant may also be included in any of these formulations.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated naphthalene. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20. Certain components included in a pharmaceutical composition of the present disclosure may be considered as a pharmaceutically acceptable carrier or an excipient.

A pharmaceutical composition of the present disclosure can be administered by a variety of methods known in the art. The route and/or mode of administration may vary depending upon the desired results. In some embodiments, the administration is intratumoral, intraventricular, intravenous, intramuscular, intraperitoneal, subcutaneous, parenteral, spinal, or epidermal. In some embodiments, the pharmaceutically acceptable carrier is suitable for intratumoral, intraventricular, intravenous, intramuscular, intraperitoneal, subcutaneous, parenteral, spinal, or epidermal administration (e.g., by injection or infusion).

A CD7 CAR-modified INKT cell or cell population (e.g., a CD3⁺iTCR(Vα24-Jα18)⁺ or CD3⁺Vα24⁺ INKT cell or cell population) may be administered alone or in combination with at least one additional therapeutic agent (e.g., an anti-cancer agent, a standard-of-care agent for the particular cancer being treated, etc.), and may be administered in any acceptable formulation, dosage, or dosing regimen. When administered in combination with an additional therapeutic agent, the additional therapeutic agent may be administered according to its standard dosage and/or dosing regimen. Alternatively, the additional therapeutic agent may be administered at a higher or lower amount and/or frequency, as compared to its standard dosage and/or dosing regimen. In some embodiments, the additional therapeutic agent is administered at a lower amount and/or frequency.

As used herein, the term “agent” refers to a chemical compound, a mixture of chemical compounds, a biological macromolecule, or an extract made from biological materials. The term “therapeutic agent” refers to an agent that is capable of modulating a biological process and/or has biological activity. The CD7 CAR-modified INKT cells and cell populations described herein are exemplary therapeutic agents. Additional therapeutic agents (e.g., those which may be administered in combination with a CD7 CAR-modified INKT cell or cell population described herein) may comprise any active ingredients suitable for the particular indication being treated (e.g., a cancer), e.g., those with complementary activities that do not adversely affect each other.

Typically, a therapeutically effective dose of a CD7 CAR-modified INKT cell or cell population is employed in the pharmaceutical compositions of the present disclosure. The CD7 CAR-modified INKT cell or cell population may be formulated into a pharmaceutically acceptable dosage form by conventional methods known in the art.

Dosage regimens for a CD7 CAR-modified INKT cell or cell population alone or in combination with at least one additional therapeutic agent may be adjusted to provide the optimum desired response (e.g., a therapeutic response). For example, a single bolus of one or both agents may be administered at one time, several divided doses may be administered over a predetermined period of time, or the dose of one or both agents may be proportionally decreased or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier.

Dosage values for compositions comprising a CD7 CAR-modified INKT cell or cell population and/or any additional therapeutic agent(s), may be selected based on the unique characteristics of the active agent(s), and the particular therapeutic effect to be achieved. A physician or veterinarian can start doses of the CD7 CAR-modified INKT cell or cell population employed in the pharmaceutical composition at levels lower than that required to achieve the desired therapeutic effect and gradually increase the dosage until the desired effect is achieved. In general, effective doses of the compositions of the present disclosure, for the treatment of a cancer may vary depending upon many different factors, including means of administration, target site, physiological state of the subject, whether the subject is human or an animal, other medications administered, and whether treatment is prophylactic or therapeutic. The selected dosage level may also depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors. Treatment dosages may be titrated to optimize safety and efficacy.

As used herein, the terms “therapeutically effective dose” and “therapeutically effective amount” are used to refer to an amount sufficient to measurably decrease at least one symptom or measurable parameter associated with a medical condition or infirmity, to normalize body functions in a disease or disorder that results in the impairment of specific bodily functions, or to provide improvement in, or slow the progression of, one or more clinically measured parameters of a disease. A therapeutically effective amount may, for example, be sufficient to treat, prevent, reduce the severity, delay the onset, and/or reduce the risk of occurrence of one or more symptoms of a cancer. A therapeutically effective amount, as well as a therapeutically effective frequency of administration, can be determined by methods known in the art and discussed herein. In some embodiments of the compositions and methods described herein, a CD7 CAR-modified INKT cell or cell population is administered in an amount that is therapeutically effective when administered as a single agent. In some other embodiments, a CD7 CAR-modified INKT cell or cell population and at least one additional therapeutic agent is each administered in an amount that is therapeutically effective when the agents are used in combination. In some embodiments, a therapeutically effective amount of a CD7 CAR-modified INKT cell or cell population is the amount required to kill a cancer cell population or a portion thereof in a subject. In some embodiments, a therapeutically effective amount of a CD7 CAR-modified INKT cell or cell population is the amount required to reduce or slow the expansion of a cancer cell population in a subject. In some embodiments, a therapeutically effective amount of a CD7 CAR-modified INKT cell or cell population is the amount required to reduce or slow the growth of a tumor in a subject.

In some embodiments, a therapeutically effective amount of a CD7 CAR-modified INKT cell or cell population is about 1×10⁷ to about 5×10⁹ cells. In some embodiments, a therapeutically effective amount of a CD7 CAR-modified iNKT cell or cell population is about 1×10⁷, about 2×10⁷, about 3×10⁷, about 4×10⁷, about 5×10⁷, about 6×10⁷, about 7×10⁷, about 8×10⁷, or about 9×10⁷ cells. In some embodiments, a therapeutically effective amount of a CD7 CAR-modified INKT cell or cell population is about 1×10⁸, about 2×10⁸, about 3×10⁸, about 4×10⁸, about 5×10⁸, about 6×10⁸, about 7×10⁸, about 8×10⁸, or about 9×10⁸ cells. In some embodiments, a therapeutically effective amount of a CD7 CAR-modified INKT cell or cell population is about 1×10⁹, about 2×10⁹, about 3×10⁹, about 4×10⁹, or about 5×10⁹ cells. In some embodiments, a therapeutically effective amount of a CD7 CAR-modified INKT cell or cell population is less than about 1×10⁷ cells. In some embodiments, a therapeutically effective amount of a CD7 CAR-modified INKT cell or cell population is more than about 5×10⁹ cells. In some embodiments, a cell, cell population, or pharmaceutical composition is administered to a subject on a single occasion. In some embodiments, a cell, cell population, or pharmaceutical composition is administered to a subject on multiple occasions (e.g., hourly, daily, weekly, bi-weekly, monthly, or yearly).

A therapeutically effective dose of a CD7 CAR-modified INKT cell or cell population described herein generally provides therapeutic benefit without causing substantial toxicity. Toxicity and therapeutic efficacy of a CD7 CAR-modified INKT cell or cell population can be determined by standard pharmaceutical procedures, e.g., in cell culture or in animal models. Cell culture assays and animal studies can be used to determine the LD50 (the dose lethal to 50% of a population) and the ED50 (the dose therapeutically effective in 50% of a population). The dose ratio between toxic and therapeutic effects is the therapeutic index, which can be expressed as the ratio LD50/ED50. In some embodiments, a CD7 CAR-modified INKT cell or cell population exhibits a high therapeutic index. The data obtained from cell culture assays and animal studies can be used in formulating a range of dosages suitable for use in humans. In some embodiments, the dosage lies within a range of circulating concentrations that include the ED50 with minimal or no toxicity. The dosage may vary within this range depending upon a variety of factors, e.g., the dosage form employed, the route of administration utilized, the condition of the subject, and the like. The exact formulation, route of administration, and dosage can be chosen by an attending physician in view of the subject's condition.

In some embodiments, a CD7 CAR-modified INKT cell, cell population, or pharmaceutical composition is administered on a single occasion. In some embodiments, a CD7 CAR-modified INKT cell, cell population, or pharmaceutical composition is administered on multiple occasions. Intervals between single dosages can be, e.g., hourly, daily, weekly, bi-weekly, monthly, or yearly. Intervals can also be irregular, based on measuring levels of the administered agent (e.g., a CD7 CAR-modified iNKT cell or cell population) in the subject in order to maintain a relatively consistent concentration of the agent. The dosage and frequency of administration of a CD7 CAR-modified INKT cell or cell population may also vary depending on whether the treatment is prophylactic or therapeutic. In prophylactic applications, a relatively low dosage may be administered at relatively infrequent intervals over a long period of time. Some subjects may continue to receive treatment for the rest of their lives. In therapeutic applications, a relatively higher dosage at relatively shorter intervals is sometimes required until progression of the disease is reduced or terminated, and/or until the subject shows partial or complete amelioration of one or more symptoms of disease. Thereafter, the subject may be administered a lower, e.g., prophylactic, dosage regime.

In some embodiments, kits and articles of manufacture for use in the therapeutic and prophylactic applications described herein are also provided. In some embodiments, the present disclosure provides a kit or article of manufacture comprising a CD7 CAR-modified INKT cell or cell population. In some embodiments, the kit or article of manufacture further comprises one or more additional components, including but not limited to: instructions for use; other reagents, e.g., a therapeutic agent (e.g., an anti-cancer agent); devices, containers, or other materials for preparing the CD7 CAR-modified INKT cell or cell population for administration; pharmaceutically acceptable carriers; and devices, containers, or other materials for administering the CD7 CAR-modified INKT cell or cell population to a subject. Instructions for use can include guidance for therapeutic applications including suggested dosages and/or modes of administration, e.g., in a subject having or suspected of having a cancer. In some embodiments, the kit comprises a CD7 CAR-modified INKT cell or cell population, and instructions for use of the CD7 CAR-modified INKT cell or cell population in treating and/or preventing a cancer.

### EXAMPLES

The following examples provide illustrative embodiments of the disclosure. The examples provided do not in any way limit the disclosure.

### Materials and Methods

Cell Culture: K562 (chronic myelogenous leukemia, CML), Daudi (B-cell Burkitt's lymphoma), Raji (B-cell Burkitt's lymphoma), Nalm-6 (B-cell precursor leukemia), EBV-B (Epstein-Barr virus transformed B cell line), HSB2 (T-cell acute lymphoblastoid leukemia, T-ALL), Jurkat (T-ALL), MOLT13 (T-ALL), KG1 (acute myeloid leukemia, AML), Kasumi-1 (AML), Kasumi-6 (AML), Molm13 (AML), MV4:11 (AML), U937 (AML), SaOS2 (osteosarcoma), Rh30 (alveolar rhabdomyosarcoma), and TC71 (Ewing sarcoma) cell lines, as well as their luciferase-expressing counterparts, were maintained in RPMI 1640 medium (Corning, Cat No. 10-040-CM) supplemented with 10% heat-inactivated fetal bovine serum (FBS, Corning, Cat No. 35-015-CV), 2 mM L-glutamine (Corning, Cat No. 25-005-CI), 50 U/mL penicillin and 50 μg/mL streptomycin (Corning, Cat No. 30-002-CI). Kasumi-1 (AML) and Kasumi-6 (AML) were maintained in RPMI 1640 medium supplemented with 20% heat-inactivated FBS, 2 mM L-glutamine, 50 U/mL penicillin and 50 μg/mL streptomycin, and 2-20 ng/ml human granulocyte-macrophage colony-stimulating factor (GM-CSF, Leukine, Partner Therapeutics). BE (2) C (neuroblastoma) and SKNFI (neuroblastoma) were maintained in 1:1 mixture of Eagle's Minimum Essential Medium (ATCC, Cat No. 30-2003) and F12 medium (ATCC, Cat No. 30-2006) supplemented with 10% FBS (Corning, Cat No. 35-015-CV), and Dulbecco's Modified Eagle's Medium (DMEM) (Corning, Cat No. 10-013-CM) supplemented with 10% FBS, 0.1 mM Non-Essential Amino Acid (Corning, Cat No. 45000-700).

K562, Daudi, Raji, Nalm-6, BE (2) C, and SKNFI were purchased from ATCC. EBV-transformed B (EBV-B) cell line was provided by the Ludwig Cancer Research Branch in Belgium. HSB2, Jurkat, MOLT13, KG-1, Kasumi-1, Kasumi-6, Molm-13, MV4:11, U937, Rh30, and SaOs2 were provided by the University of Minnesota. TC71 was provided by the University of Utah. Authentication of HSB2-, Jurkat-, MOLT13-, and KG1-expressing luciferase and NGFR (hfflucN) was performed for ten short tandem repeat (STR) loci plus the gender determining locus, Amelogenin by Oregon Health Science University Integrated Genomics Laboratory.

### Peripheral Blood Mononuclear Cell Isolation, CAR-T Production and Culture:

Peripheral blood mononuclear cells (PBMCs) were isolated from whole blood units purchased from New York Blood Center using Ficoll Paque Plus (Cytiva, Cat No. GE17-1440-03) and cryopreserved. PBMCs were thawed and cultured at 1×10⁶ cells per well in a 24-well plate in human T-cell medium composed of RPMI 1640 (Corning, Cat No. 10-040-CM), 10% FBS (Corning, Cat No. 35-015-CV), 2 mM L-glutamine (Corning, Cat No. 25-005-CI), 50 μM 2-mercaptoethanol (2-ME, Sigma-Aldrich, Cat No. 60-24-2), 100 IU/mL penicillin and 100 μg/mL streptomycin (Corning, Cat No. 30-002-CI) with Dynabeads Human T-Activator CD3/CD28 (Invitrogen, Cat No. 11131D). On day 3, cultures were transduced with CD19, ROR1, CD7 CAR lentiviruses (Multiplicity of Infection (MOI)=9-20) on retronectin (Takara Bio, Cat No. T100B)-coated 24-well plates by centrifugation at 1,902 g (3000 rpm), 32° C. for 2 hours and incubated at 37° C. for 48 h. After removal of CD3/CD28 beads, transduced T cells or untransduced mock T cells were cultured in human T cell medium supplemented with human IL-2 (50 IU/mL, Proleukin, Novartis Pharmaceuticals), IL-7 (10 ng/ml, Peprotech, Cat No. 200-07), and IL-15 (10 ng/ml, Peprotech, Cat No. 200-15). Cell numbers were determined on a hemocytometer by trypan blue staining on day 11 and 16.

INKT Cell Isolation, CAR Transduction and Expansion: INKT cells (1-2×10⁵ per well) were isolated from fresh PBMCs using anti-iNKT microbeads (Miltenyi Biotec, Cat No. 130-094-842) that positively select human cells expressing TCRVα24-Jα18 and cultured in a 48-well plate with irradiated (40 Gy) autologous PBMC negative fraction (3-×10⁶ per well) post iNKT isolation in 1 mL of human T cell medium supplemented with α-GalCer (200 ng/ml, DiagnoCine, Cat No. KRN7000; FlycoFineChem, Cat No. FC-070) or 7DW8-5 Glycolipid Analog (200 ng/ml, provided by Dr. Moriya Tsuji at Columbia University; DiagnoCine, Cat No. 7DW8-5), 250 IU/mL IL-2 (Proleukin, Novartis Pharmaceuticals), and 10 ng/ml IL-15 (Peprotech, Cat No. 200-15). On day 3 or 4, INKT cell cultures were transduced with CAR lentivirus (MOI=20-200) on retronectin (Takara Bio, Cat No. T100B)-coated 24-well plates by centrifugation at 1,902 g (3000 rpm), 32° C. for 2 hours. Transduced INKT cells were transferred to 48-well plates after 48 hours, fed with human T-cell medium supplemented with IL-2 and IL-15, and divided into new wells when they were confluent. At 2-3 weeks, expansion of INKT cells was carried out initially in a 24-well plate and then transferred to a T75 flask. Briefly, INKT cells (5×10⁵ per well) were co-cultured with irradiated autologous PBMC negative fraction and/or whole PBMCs (termly auto) or mixed PBMCs from 3-5 allogenic donors (3.5×10⁶ per well, 40 Gy) and irradiated EBV-B cells (5×10⁵ per well, 80 Gy; termly allo) in 2 mL per well of a 24-well plate of human T-cell medium supplemented with α-GalCer or 7DW8-5 (100-200 ng/ml), IL-2 (250 IU/mL), and IL-15 (10 ng/ml). Expansion of INKT cells were also carried out in a T25 flask by co-culture of INKT cells (5×10⁵ per flask), irradiated mixed PBMCs from 3-5 allogenic donors (3×10⁷ per flask, 40 Gy) and irradiated EBV-B cells (3×10⁶ per flask, 80 Gy) in 25 mL of human T-cell medium supplemented with either α-GalCer or 7DW8-5 (100-200 ng/ml), IL-2 (250 IU/mL), and IL-15 (10 ng/ml) or anti-human CD3 monoclonal antibody (OKT3, 30 ng/ml, Miltenyi Biotec, Cat NO. 130-093-387), IL-2 (50 IU/mL), IL-7 (10 ng/ml), and IL-15 (10 ng/mL). Expanded INKT cells were cryopreserved at 8×10⁷ cells per vial in 1 mL of 90% FBS and 10% dimethyl sulfoxide (DMSO, MPBio, SKU NO. 021960559). αGalCer or 7DW8-5 was dissolved in DMSO at 1 mg/ml or 4 mg/mL, respectively and stored at −20° C. after heating for 10 min at 60-80° C. water beaker, vortexing for 30 sec, and aliquoting at 10 μl or 2 μl per tube. When use, αGalCer (10 μl per tube) or 7DW8-5 (2 μl per tube) was heated for 10 min at 60-80° C. water beaker, vortexed for 30 sec, and diluted at 100 μg/mL with T-cell medium.

Lentiviral Production and Tumor Cell Transduction: An HIV-1-based bidirectional vector expressing humanized firefly luciferase and NGFR (hfflucN) was constructed as previously described (Huang et al., Mol Ther. 2008; 16 (3): 580-9; Huang et al., PLOS ONE. 2015; 10 (7): e0133152). A lentiviral vector expressing a CD7 CAR, CD19 CAR, and ROR1 comprising a single chain variable region (scFv) of an anti-CD7 (SEQ ID NO. 36), -CD19 (SEQ ID NO. 37), and -ROR1 (SEQ ID NO. 38) antibody, a CD8a hinge and transmembrane region, and an intracellular domain of 4-1BB and a CD3 zeta chain was constructed based on a lentiviral vector previously described (Milone et al., Mol Ther. 2009; 17 (8): 1453-64; Tammana et al., Hum Gene Ther. 2010; 21:75-86; Baskar et al., mAbs. 2012; 4 (3): 349-361; Huang et al., PLOS ONE. 2015; 10 (7): e0133152). CD7 CAR was generated by using commercial gene synthesis of an anti-CD7 single-chain variable fragment (scFv) sequence found in patent WO2003051926A2 (Cooper et al., Leuk. 2018; 32 (9): 1970-1983). Lentiviral supernatants were harvested 48 h and 96 h post transfection in Lenti-X 293T cell line (Takara Bio, Cat No. 632180) with 4 plasmids (pLVCARsin, pMDLg/pRRE, PRSV-REV, pMD2.G) and Lipofectamine 2000 (ThermoFisher, Cat No. 11668019) and concentrated using Lenti-X Concentrator (Takara Bio, Cat. No. 631231). Viral titer was determined in Lenti-X 293T cell line and ranged from 2.3×10⁷-2×10⁸ transducing units/mL. Leukemia cell lines were spin transduced at 1170 g at 32° C. for 1 h with the hfflucN lentivirus in the presence of polybrene (8 μg/mL) and then sorted by FACS or enriched by biotin anti-human CD271 (NGFR, BD Biosciences, Cat No. 557195) and anti-biotin microbeads (Miltenyi Biotec, Cat No. 130-090-485) or CD271 microbeads (Miltenyi Biotec, Cat No. 130-099-023) for NGFR expression. All transduced cell lines were verified by flow cytometric analysis of NGFR expression and hffluc bioluminescence activity using a Synergy 2 microplate reader (BioTek).

Luciferase-Based Killing Assay: αGalCer or DMSO pulsed target cells were prepared by incubating target cells (2×10⁵/mL) at 37° C., 5% CO2 with αGalCer (200 ng/ml) or DMSO (1:500 of 10% stock) overnight and used for cytotoxicity assays after spinning to remove medium containing αGalCer or DMSO. Luciferase-expressing target cells (1×104 in 50 μL per well) were incubated with 50 μL of INKT cells per well at different effector T cell: target cell (E/T) ratios in quadruplicate in a 96-well flat-bottom white polystyrene microplate (Corning, Cat No. 3912). A spontaneous or maximal killing was set up by adding 50 μL per well of culture medium or 1% Triton X-100 instead of INKT cells, respectively. After a standard 4 h or 16 h (as indicated) incubation at 37° C., 10 μL of D-luciferin (1:10 of 30 mg/ml stock) or 50 μL (1:50 of 30 mg/mL) (Caliper Life Sciences, Cat No. XR-1001) was added to each well. Luciferase activity was measured using a Synergy 2 microplate reader (BioTek). Percent specific lysis was calculated as follows:

$$\% Specific lysis = ( spontaneous death RLU - sample RLU ) / ( spontaneous death RLU - maximal death RLU ) \times 100.$$

$$RLU = Relative Luminescence Units .$$

Antibodies and Flow Cytometry: FITC anti-human TCR Vα24-Jα18 (INKT cell, clone 6B11 recognizing the invariant CDR3 region of TCRVα24-Jα18 and TCRVα24-JQ, Cat No. 342906), PE anti-human CD1d (clone 51.1, Cat No. 350306), FITC anti-human PD1 (clone EH12.2H7, Cat No. 329904), APC anti-human CD3 (clone UCHT1, Cat No. 300439), PE anti-His Tag (clone J095G46, Cat No. 362603), PE Streptavidin (Cat No. 405204), PE mouse IgG1, k isotype control (clone MOPC-21, Cat No. 981804), APC mouse IgG1, k isotype control (clone MOPC-21, Cat No. 981806), PE mouse IgG2a, k isotype control (clone MOPC-173, Cat No. 400246), and PE mouse IgG2b, k isotype control (clone MPC-11, Cat No. 400314) were purchased from BioLegend. PE anti-human CD2 (clone RPA-2.10, Cat No. 555327), APC anti-human CD3 (clone UCHT1, Cat No. 555335), PE anti-human CD5 (clone UCHT2, Cat No. 555353), PE anti-human CD4 (clone PRA-T4, Cat No. 555347), APC anti-human CD4 (clone PRA-T4, Cat No. 555349), PE anti-human CD8 (clone HIT8a, Cat No. 555635), APC anti-human CD8 (clone RPA-T8, Cat No. 555369), V450 anti-human CD7 (clone M-T701, Cat No. 642916), PE anti-human CD7 (clone M-T701, Cat No. 555361), PE anti-human CD271 (NGFR) (clone C40-1457, Cat No. 557196), PE mouse IgG1, k isotype control (clone MOPC-21, Cat No. 555749), APC mouse IgG1, k isotype control (clone MOPC-21, Cat No. 555751), and V450 mouse IgG1, k isotype control (clone MOPC-21, Cat No. 560373) were purchased from BD Biosciences. Biotinylated human CD19 protein (Cat No. 11880-H08H-B), biotinylated human ROR1 protein (Cat. No. 13968-HCCH1-B), and Human CD7 protein (His Tag, Cat. No. 11028-H08H) were purchased from SinoBiological. Surface CAR expression was detected with biotinylated human CD19 protein or biotinylated human ROR1 and PE streptavidin or human CD7 protein His Tag and PE anti-His Tag or Alexa Fluor 647 (AF-647)-conjugated F(ab′) 2 fragments of goat anti-mouse IgG F(ab′)₂ (anti-CAR, Jackson ImmunoResearch, Cat No. 115-606-006) and Alexa Fluor 647-conjugated F(ab′)₂ fragments of ChromPure goat IgG isotype control (Cat No. 015-600-006). Flow cytometric analysis was performed on a BD FACSCelesta or BD Accuri C6 cytometer. Data were analyzed with Flowjo software 7.2.2 or V10.

Cytokine Release Assays: Cytokine release assays were performed by co-culturing 1-2×10⁵ T cells with 2×104 target cells per well in duplicate in 96-well flat-bottom plates. After 24 h, supernatants were assayed using a LEGEND MAX Human IFN-γ ELISA kit (BioLegend, Cat No. 430107).

In Vivo Anti-Tumor Assays: NOD.Cg-Prkdc^{scid}IL2rg^{tm1Wjl/SzJ} (NSG, stock number 005557) mice were purchased from the Jackson Laboratory (Bar Harbor, ME) and housed in the specific pathogen free facility at New York Medical College. To establish T-ALL and AML xenograft models, 6-7-week-old NSG mice were inoculated intravenously (i.v.) with 5×10⁵ HSB2-hfflucN or KG1-hfflucN cells on day-2. On day 0, mice were examined for human leukemia engraftment by bioluminescent imaging (BLI) using the Perkin Xenogen IVIS Spectrum Imaging System. Mice with T-ALL or AML were randomly divided into three or two groups (n=5 per group) based on imaging intensity. On day 0, 3, and 6, mice bearing with HSB2-hfflucN T-ALL were infused i.v. with PBS, thawed mock iNKT55 cells, and thawed CD7 CAR-INKT55 cells (5×10⁶/0.2 mL per mouse). Mice bearing with KG1-hfflucN AML were infused i.v. with thawed mock iNKT55 and CD7 CAR-INKT55 on day 0 and 3. Mock INKT55 and CD7 CAR-INKT55 cells were thawed from a liquid nitrogen dewar, washed twice with human T-cell medium and PBS, and resuspended in PBS. Mice were also injected intraperitoneally (i.p.) every three days for two weeks with cytokine mixture (IL-2 2,000 IU/mouse, IL-15 2 μg/mouse) or PBS (Tian et al., J Clin Invest. 2016; 126 (6): 2341-55; Xu et al., Clin Cancer Res. 2019; 25 (23): 7126-38). Tumor BLI was performed on day 3, 6, 10, 17 and 24 in mice with T-ALL or on day 0, 3, 6, 10, 17, 24, 31, 38, 45 in mice with AML after the first T-cell infusion. BLI was carried out under isoflurane anesthesia after intraperitoneal injection of D-luciferin (Caliper Life Sciences, Cat No. XR-1001). Images were collected and analyzed using the IVIS Imaging System and Living Image 4.7.3 software (PerkinElmer). A constant region-of-interest (ROI) was drawn over the entire mouse body and the intensity of the signal measured as total photon flux normalized for exposure time and surface area and expressed in units of photons/sec/cm²/steradian (p/sec/cm²/sr). Mice were then monitored for survival without imaging manipulations.

Statistical analysis: Data were calculated as mean±standard deviation (SD) and analyzed using One-Way ANOVA analysis and followed by Tukey's Honestly Significant Difference (Tukey's HSD) post-hoc test for pairwise comparisons. Statistical significance in Kaplan-Meier survival curves was assessed with the Mantel-Cox log-rank test. All p values were calculated using Prism 7 software (GraphPad).

### Results

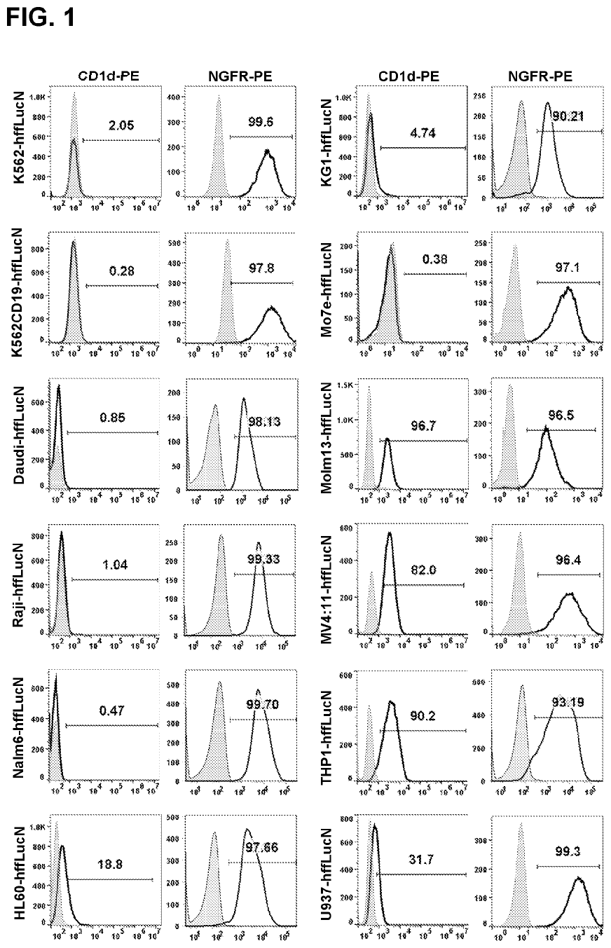

FIG. 1 shows that CD1d was expressed in most AML cell lines including HL60, KG1, Molm13, MV4:11, THP-1, and U937 but not in Mo7e AML cell line. CD1d expression in KG1 was weakly positive. CD1d expression in B-cell tumor cell lines (Daudi, Raji, Nalm6) and CML cell line (K562, K562CD19) was negative. All luciferase-transduced tumor cell lines expressed high levels (>90%) of NGFR, indicating that they all may express luciferase.

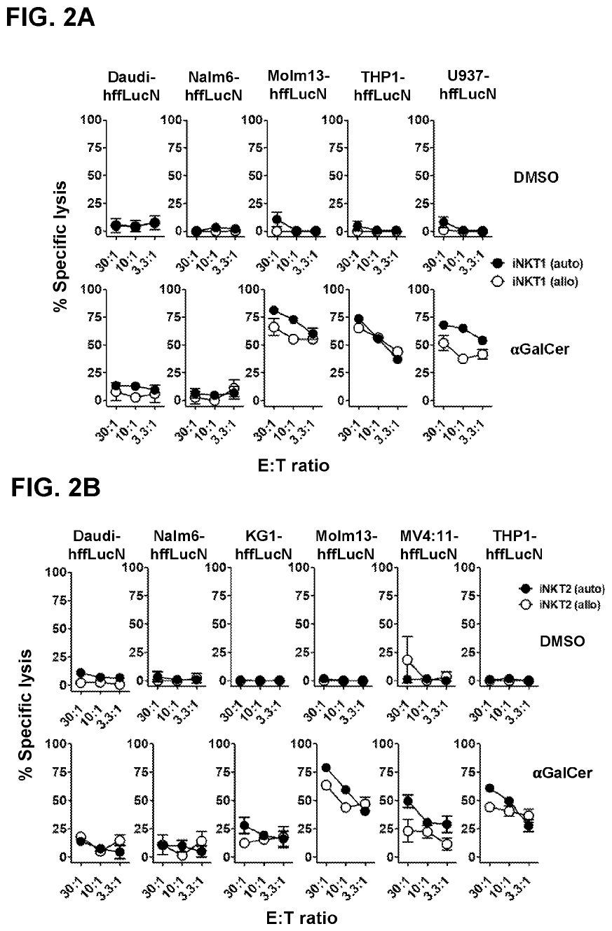

FIG. 2A shows that iNKT1 (allo) cells expanded with irradiated allogeneic PBMCs and EBV-B cells, and αGalCer antigen specifically lysed αGalCer sensitized CD1d⁺ (Molm13, THP-1, U937) but not CD1d⁺ (Daudi, Nalm6) target cells compared to iNKT1 (auto) cells expanded with irradiated autologous PBMC negative fraction and/or whole PBMCs and αGalCer, which is a common method in INKT cell expansion. Control DMSO sensitized CD1d⁺ and CD1d target cells were not lysed by iNKT1 (auto) and iNKT1 (allo).

FIG. 2B shows that iNKT2 (allo) cells expanded with irradiated allogeneic PBMCs and EBV-B cells, and αGalCer antigen specifically lysed αGalCer sensitized CD1d⁺ (KG1, Molm13, MV4:11, THP-1) but not CD1d (Daudi, Nalm6) target cells compared to INKT2 (auto) cells expanded with irradiated autologous PBMC negative fraction and/or whole PBMCs and αGalCer. These results based on FIG. 2A and FIG. 2B suggest that iNKT cells expanded with irradiated allogeneic PBMCs and EBV-B cells, and αGalCer antigen remain αGalCer-CD1d-specific compared to iNKT cells expanded with irradiated autologous PBMC negative fraction and/or whole PBMCs and αGalCer.

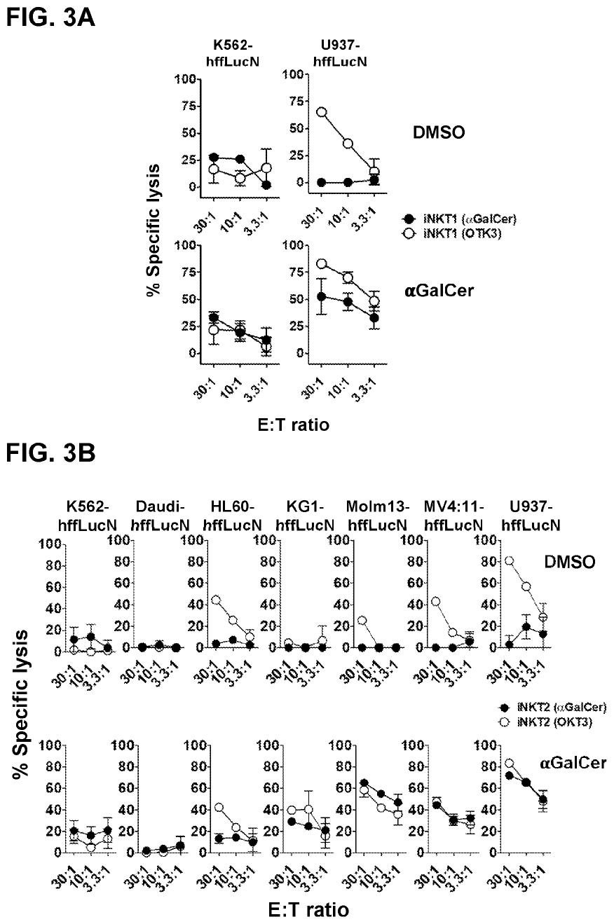

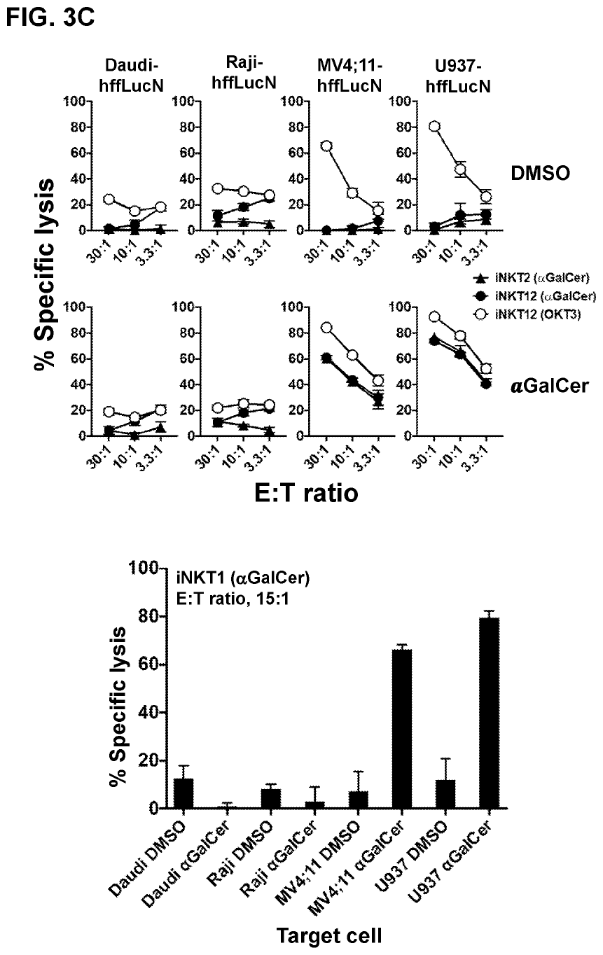

FIG. 3A shows that iNKT1 (OKT3) cells expanded with irradiated allogeneic PBMCs and EBV-B cells, and soluble anti-CD3 monoclonal antibody (OKT3) surprisingly recognized CD1d⁺ (U937) target cells independent of αGalCer. As control, INKT1 (αGalCer) cells expanded with irradiated allogeneic PBMCs and EBV-B cells, and αGalCer antigen recognized αGalCer but not DMSO treated CD1d⁺ target cells. Both INKT1 (OKT3) and INKT1 (αGalCer) did not recognize CD1d⁻ K562 cells regardless of αGalCer or DMSO treatment.

FIG. 3B shows that iNKT2 (OKT3) cells expanded with irradiated allogeneic PBMCs and EBV-B cells, and soluble anti-CD3 monoclonal antibody (OKT3) recognized CD1d⁺ (HL60, Molm13, MV4:11, U937) target cells independent of αGalCer. As control, INKT2 (αGalCer) cells expanded with irradiated allogeneic PBMCs and EBV-B cells, and αGalCer antigen recognized αGalCer but not DMSO treated CD1d⁺ target cells. Both INKT2 (OKT3) and iNKT2 (αGalCer) did not recognize CD1d (K562, Daudi) cells regardless of αGalCer or DMSO treatment. A weak recognition of DMSO treated KG1 cells by INKT2 (OKT3) may likely be due to a low-level expression of CD1d in KG1 cells (FIG. 1).

FIG. 3C shows that iNKT12 (OKT3) cells expanded with irradiated allogeneic PBMCs and EBV-B cells, and soluble anti-CD3 monoclonal antibody (OKT3) recognized CD1d⁺ (MV4:11, U937) target cells independent of αGalCer. As control, INKT12 (αGalCer), INKT2 (αGalCer), and iNKT1 (αGalCer) cells expanded with irradiated allogeneic PBMCs and EBV-B cells, and αGalCer antigen recognized αGalCer but not DMSO treated CD1d⁺ target cells. INKT12 (OKT3) but not iNKT12 (αGalCer) and iNKT2 (αGalCer) also showed a low background recognition of CD1d (Daudi, Raji) cells regardless of αGalCer or DMSO treatment.

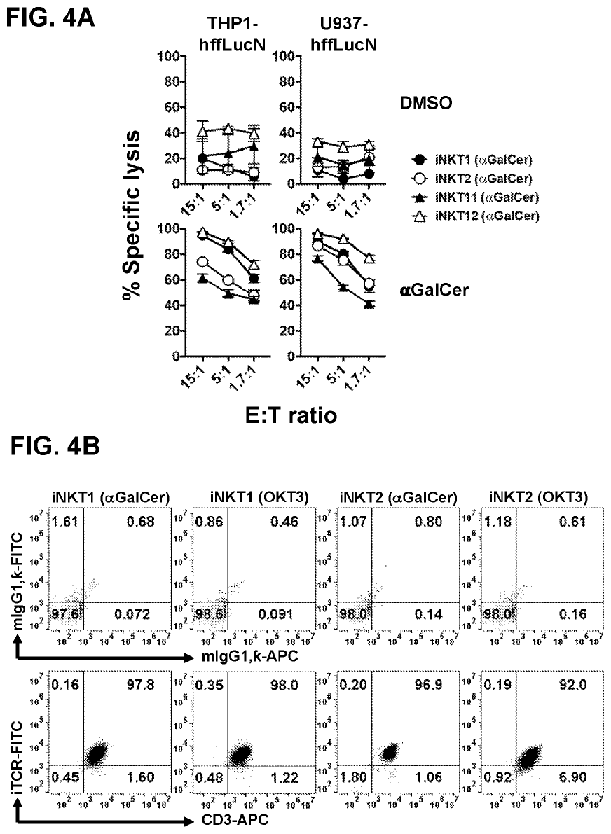

FIG. 4A shows that iNKT cells from 4 donors (INKT1, INKT2, INKT11, INKT12) expanded with irradiated allogeneic PBMCs and EBV-B cells, αGalCer, and mixed cytokines (IL-2, IL-7, and IL-15) recognized αGalCer but not DMSO treated CD1d⁺ (THP1, U937) target cells.

FIG. 4B shows that iNKT1 and iNKT2 cells expanded with OKT3 or αGalCer were more than 92% CD3⁺iTCR⁺ and phenotypically similar in terms of CD3⁺iTCR⁺ expression. All these results based on three donor-derived INKT (INKT1, INKT2, INKT12) cells expanded with OKT3 and four donor-derived INKT (INKT1, INKT2, INKT11, INKT12) expanded with αGalCer suggest that OKT3 expansion may alter the antigen specificity of INKT cells. αGalCer stimulation of INKT cells is critical in maintaining their antigen specificity.

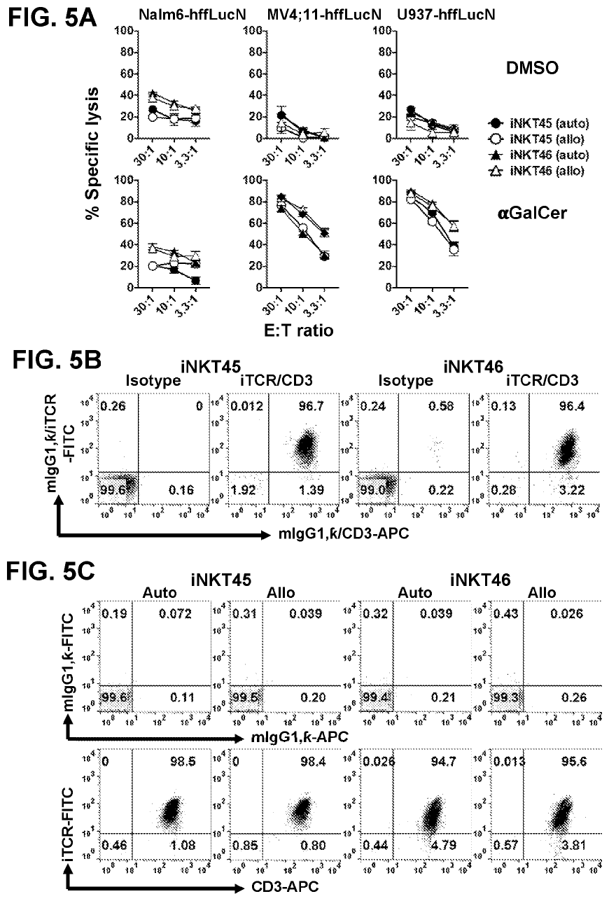

Next, we tested the function, phenotype, and expansion rate of INKT cells expanded with irradiated allogeneic PBMCs and EBV-B cells, and αGalCer antigen (termly INKT (allo)) to compare with iNKT cells expanded with irradiated autologous PBMC negative fraction and/or whole PBMCs and αGalCer (termly, INKT (auto)), all supplemented with IL-2 and IL-15. FIG. 5A shows that iNKT45 (auto), INKT45 (allo), INKT46 (auto), and iNKT46 (allo) isolated from healthy blood donor 45 and donor 46 and generated using auto or allo expansion protocols were only reactive to αGalCer but not DMSO treated CD1d⁺ (MV4:11, U937) target cells. Moreover, those INKT45 and iNKT46 indistinguishably recognized CD1d⁻×(Nalm6) target cells regardless of DMSO or αGalCer.

FIG. 5B shows that iNKT45 and iNKT46 cells after primary stimulation with irradiated autologous PBMC negative fraction and αGalCer displayed a homogenous population of more than 96% CD3⁺iTCR⁺. As shown in FIG. 5C, after secondary stimulation or expansion with irradiated autologous PBMC negative fraction and/or whole PBMCs and αGalCer or irradiated allogeneic PBMCs and EBV-B cells, and αGalCer, a homogenous population of more than 94% CD3⁺iTCR⁺ cells was demonstrated in INKT45 and INKT46 cells expanded in auto or allo conditions.

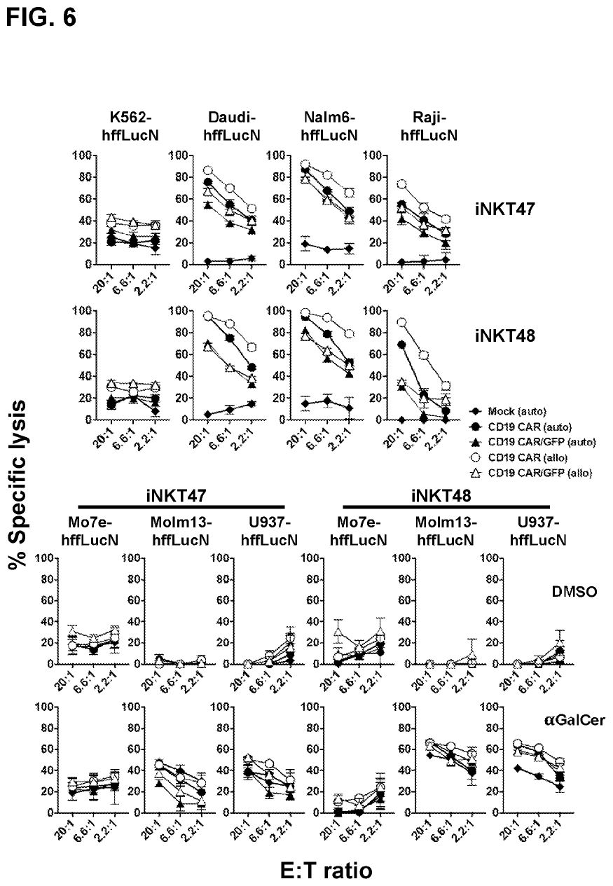

To demonstrate that iNKT cells expanded with irradiated allogeneic PBMCs and EBV-B cells, and αGalCer can be used to manufacture CAR-modified INKT cells, INKT47 and iNKT48 cells isolated from donor 47 and donor 48 were transduced via lentivirus to express CD19 CAR or CD19 CAR/GFP and expanded in auto or allo conditions. FIG. 6 shows that CD19 CAR- or CD19 CAR/GFP-modified INKT47 and INKT48 cells expanded in auto or allo conditions specifically killed CD19⁺ B-cell leukemia and lymphoma cell lines (Daudi, Nalm6, Raji) but not CD19 CML (K562) cells in an E/T ratio-dependent manner. As expected, mock iNKT47 and iNKT48 cells were incapable of killing CD19⁺ B-cell leukemia and lymphoma cell lines. Furthermore, CD19 CAR- or CD19 CAR/GFP-modified INKT47 and iNKT48 cells expanded in auto or allo conditions were reactive to αGalCer but not DMSO treated CD1d⁺ (MomI13, U937) target cells. CD1d Mo7e cells treated with DMSO or αGalCer were not recognized by all iNKT47 and iNKT48.

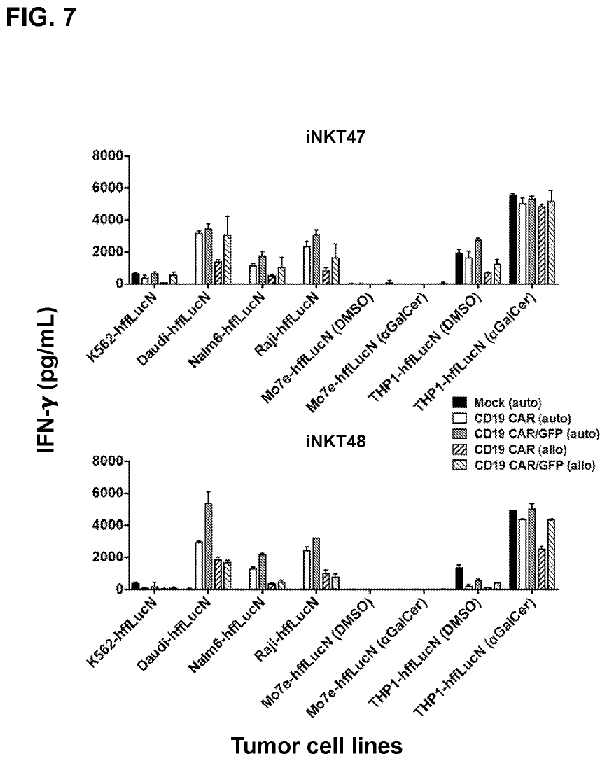

FIG. 7 shows that CD19 CAR- or CD19 CAR/GFP-modified INKT47 and iNKT48 cells expanded in auto or allo conditions specifically produced higher amounts of IFN-γ than their mock counterparts in response to CD19⁺ B-cell leukemia and lymphoma cell lines (Daudi, Nalm6, Raji) but not CD19-CML (K562) and AML (Mo7e) cells. In addition, αGalCer treated CD1d⁺ THP1 cells significantly enhanced IFN-γ production by all iNKT47 and INKT48 cells compared to DMSO treated THP1 cells, indicating that CD19 CAR-modified INKT cells expanded in allo conditions are cytotoxic and produce IFN-γ in response to CD19⁺ tumor cells while they remain specific to αGalCer.

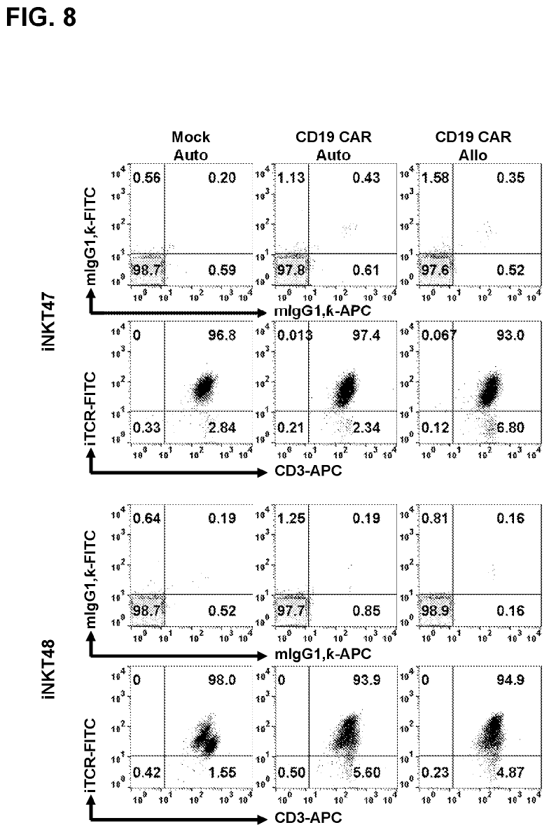

FIG. 8 shows that more than 93% of CD19 CAR-modified INKT47 and INKT48 cells expanded in auto or allo conditions expressed a CD3⁺iTCR⁺ population compared to mock counterparts, indicating that iNKT cells expanded in allo conditions possess part of their phenotypes.

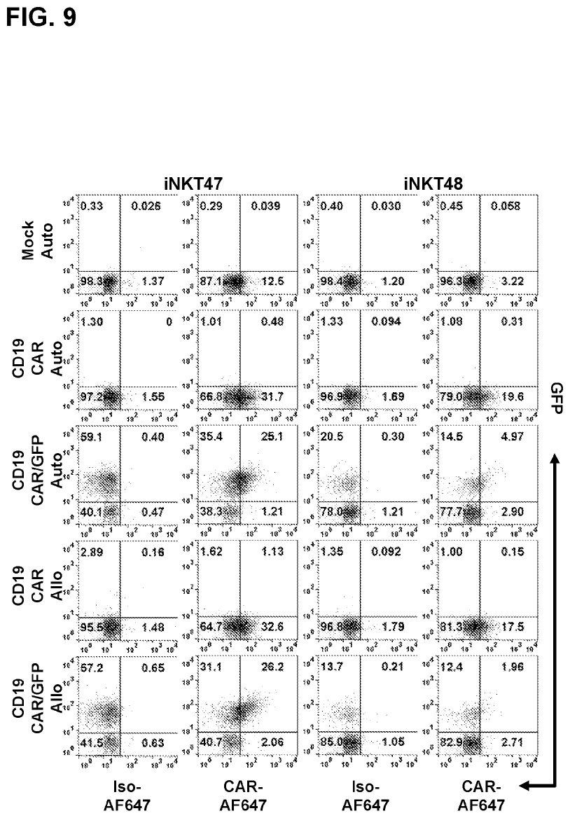

FIG. 9 shows that CD 19 CAR or CD19 CAR/GFP expression was confirmed in INKT47 and iNKT48 cells expanded in auto or allo conditions, indicating that auto or allo culture conditions did not affect the level of CAR expression.

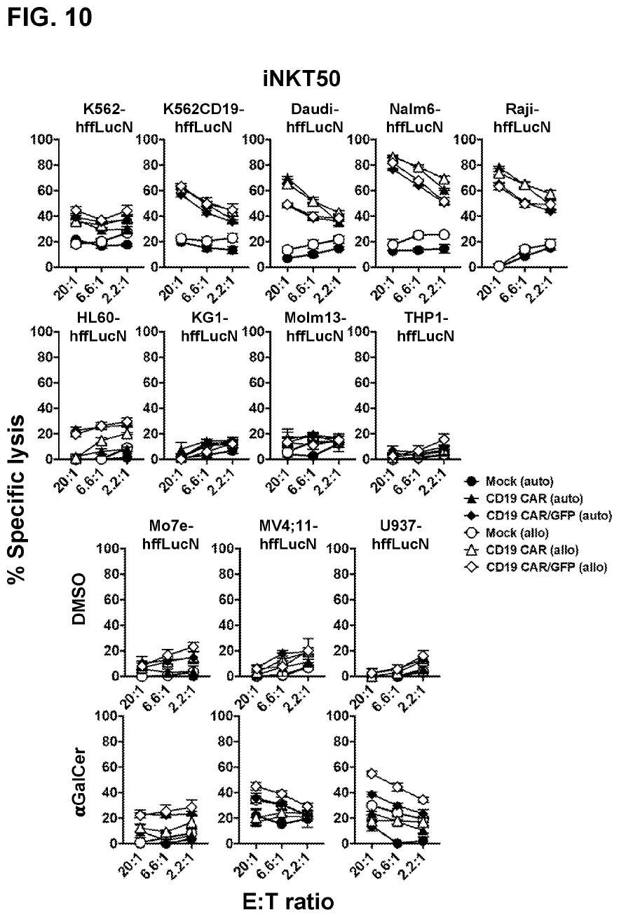

FIG. 10 shows that CD19 CAR- or CD19 CAR/GFP-modified INKT50 cells isolated from donor 50 and expanded in auto or allo conditions specifically killed CD19⁺ B-cell leukemia and lymphoma cell lines (Daudi, Nalm6, Raji) and CD19 transfected K562 (K562CD19) but not CD19-CML (K562) and AML (HL60, KG1, Molm13, THP1) cells in an E/T ratio-dependent manner. As expected, mock cells were incapable of killing CD19⁺ B-cell leukemia and lymphoma cell lines and K562CD19. Furthermore, CD19 CAR- or CD19 CAR/GFP-modified INKT50 cells expanded in auto or allo conditions were reactive to αGalCer but not DMSO treated CD1d⁺ (MV4:11, U937) target cells. CD1d⁻ Mo7e cells treated with DMSO or αGalCer were not recognized by all iNKT50 cells.

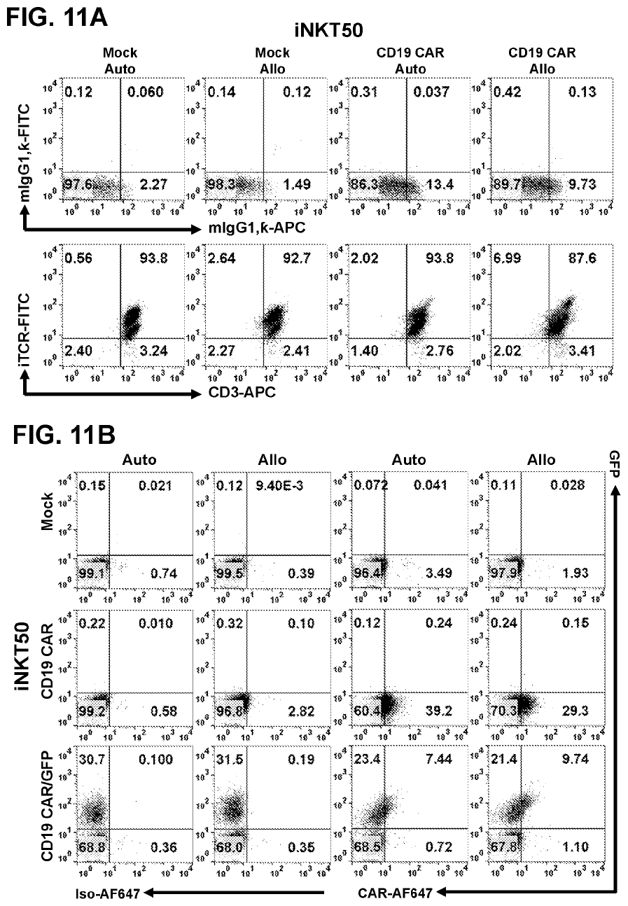

FIG. 11A shows that more than 87% of CD19 CAR-modified INKT50 cells expanded in auto or allo conditions was CD3⁺iTCR⁺ compared to mock counterparts, indicating that iNKT cells expanded in allo conditions remain phenotypically intact.

FIG. 11B shows that CD 19 CAR or CD19 CAR/GFP expression was confirmed in transduced iNKT50 but not mock cells expanded in auto or allo culture conditions, indicating that auto or allo culture conditions did not affect the level of CAR expression.

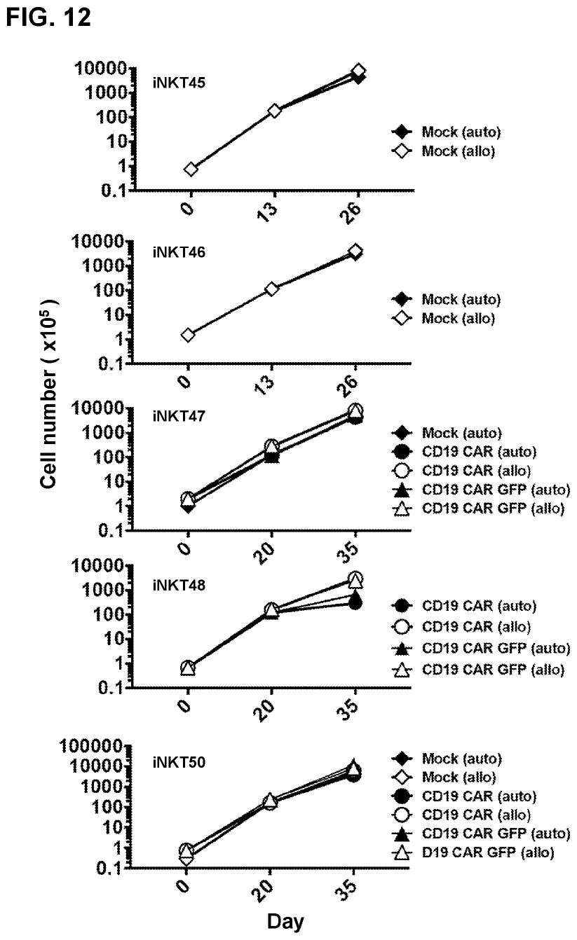

FIG. 12 shows the expansion rate of untransduced INKT45 and iNKT46 as well as CD19 CAR-modified INKT47, INKT48, and INKT50 expanded in auto or allo culture conditions. Approximately 245- and 76-fold expansion at day 13 after primary stimulation with irradiated autologous PBMC negative fraction and αGalCer was achieved in INKT45 and iNKT46, respectively. On day 26, 10,800- and 2,800-fold expansion in INKT45 and INKT46 expanded in allo conditions was obtained compared to 6,080- and 2,106-fold expansion in iNKT45 and iNKT46 cultured in auto conditions. It appears that allo culture conditions are as effective as auto culture conditions in iNKT cell expansion.

Next, we tested CD19 CAR-modified INKT cell expansion in iNKT47, INKT48, and INKT50 using auto and allo expansion conditions. As shown in FIG. 12, in the end of cultures at day 35, 4, 160- and 4,065-fold expansion in iNKT47 CD19 CAR (allo) and INKT47 CD19 CAR/GFP (allo) cells expanded in allo conditions was demonstrated respectively compared to 2,250- and 2,680-fold expansion in INKT47 CD19 CAR (auto) and INKT47 CD19 CAR/GFP (auto) expanded in auto conditions. As for iNKT48 expansion, in the end of cultures at day 35, 4,417- and 3,650-fold expansion in iNKT48 CD19 CAR (allo) and iNKT48 CD19 CAR/GFP (allo) cells expanded in allo conditions was documented respectively compared to 441- and 985-fold expansion in INKT48 CD19 CAR (auto) and iNKT48 CD19 CAR/GFP (auto) expanded in auto conditions.

We also conducted CD19 CAR-modified INKT cell expansion in INKT50 cells. As shown in FIG. 12, at day 36, 18,500-, 8,400-, and −10,680-fold expansion in INKT50 mock (allo), INKT50 CD19 CAR (allo), and iNKT50 CD19 CAR/GFP (allo) cells expanded in allo conditions was documented respectively compared to 14,666-, 5,200-, and 15,353-fold expansion in iNKT50 mock (auto), INKT50 CD19 CAR (auto), and iNKT50 CD19 CAR/GFP (auto) expanded in auto conditions. In the end of culture, 4.4×10⁸ INKT50 mock (auto), 5.5×10⁸ INKT50 mock (allo), 3.9×10⁸ INKT50 CD19 CAR (auto), 6.3×10⁸ INKT50 CD19 CAR (allo), 1.1×10⁹ INKT50 CD19 CAR/GFP (auto), and 8.0×10⁸ INKT50 CD19 CAR/GFP (allo) were generated from their starting numbers of 0.3×10⁵ (mock) and 0.75×10⁵ (CAR) isolated INKT50 cells. It appears that 5,863 (5,863±2,858, n=6)- and 4,484 (4,484±5,576, n=6)-fold expansion rate of CD19 CAR- and CD19 CAR/GFP-modified INKT cells was demonstrated in allo or auto conditions, respectively. Allo conditions may be more advantageous than auto conditions in iNKT expansion. For example, allo feeder cells can be pre-made, banked, and used for all iNKT expansion whereas auto feeders are made individually, thus likely simplifying regulatory process and reducing manufacture cost and product variation.

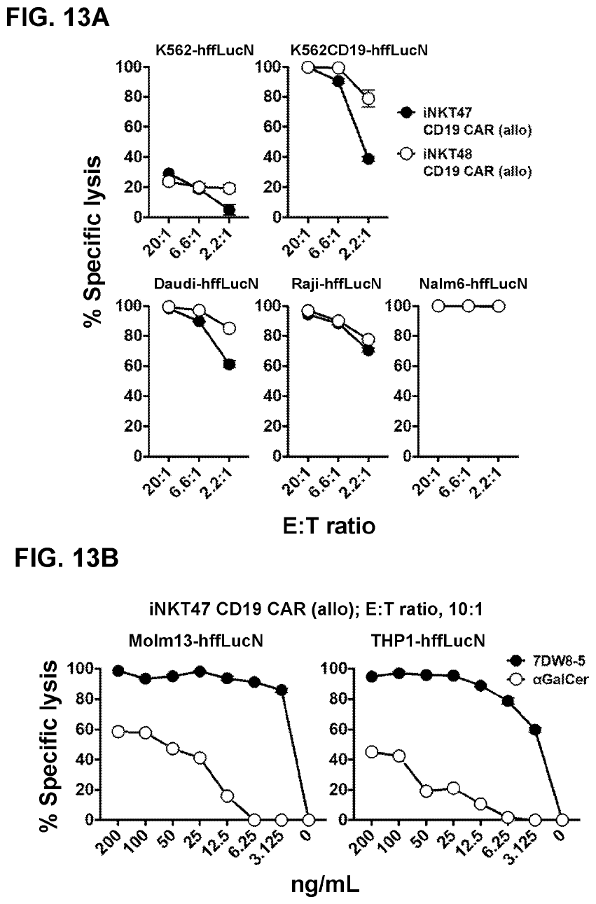

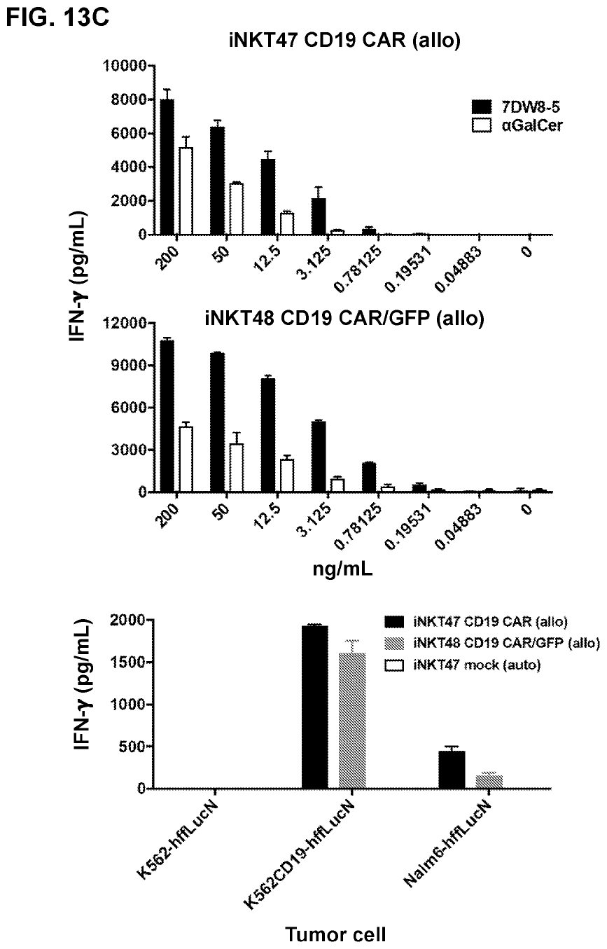

FIG. 13A shows thawed and cultured iNKT47 CD19 CAR (allo) and iNKT48 CD19 CAR (allo) were remarkably capable of killing CD19⁺ B-cell leukemia and lymphoma cells (Daudi, Raji, Nalm6) and CD19 transfected K562 (K562CD19) but not CD19-CML (K562) even at a low E/T ratio of 2.2:1 in a 16 h assay, indicating that freezing and thawing conditions minimally affect CAR-modified INKT cell cytotoxicity. In addition, FIG. 13B shows that thawed and cultured iNKT47 CD19 CAR cells (allo) killed 7DW8-5 or αGalCer treated CD1d⁺ Molm13 and THP1 target cells in a dose-dependent manner. It appears that 7DW8-5 was at least 16- and 64-fold more potent than αGalCer in sensitizing CD1d⁺ Molm13 and THP1 target cells. Moreover, FIG. 13C shows that 7DW8-5 was more potent than αGalCer in stimulating 3-month cultured INKT47 CD19 CAR (allo) and iNKT48 CD19 CAR/GFP (allo) to produce IFN-γ, confirming that 7DW8-5 exhibits a superior effect than αGalCer on INKT cell activity (Li et al., Proc Natl Acad Sci USA. 2010; 107 (29): 13010-13015). As controls, CD19 CAR-modified INKT47 cells (allo) and CD19 CAR/GFP-modified INKT48 cells (allo) but not mock iNKT47 (auto) produced IFN-γ in response to CD19⁺ target cells (K562CD19, Nalm6).

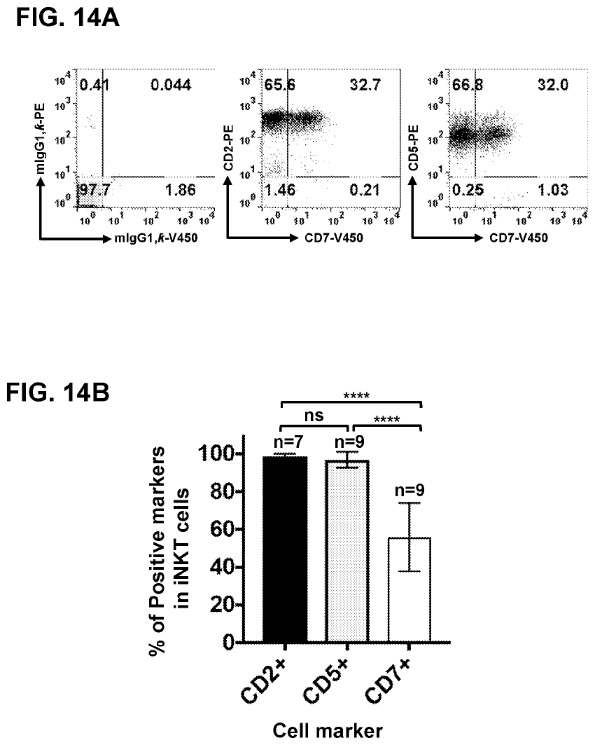

FIG. 14A shows that iNKT cells from one representative blood donor after primary stimulation with irradiated autologous PBMC negative fraction and αGalCer or 7DW8-5 and secondary expansion with irradiated allogeneic PBMCs and EBV-B cells, αGalCer or 7DW8-5, IL-2, IL-15, and/or IL-7 were unexpectedly found to display two populations of CD7⁺ (about 32%) and CD7⁻ (about 66%) subsets when compared to more than 95% of single CD2⁺ or CD5⁺ populations. This finding was surprising as it is well documented that the majority of conventional peripheral blood T and NK cells (median 90% and 97%) express CD7 (Gomes-Silva et al., Mol Ther. 2019; 27 (1): 272-280; Kim et al., JCI Insight. 2021; 6 (16);e149819).

FIG. 14B shows that approximately 56±17.96% and 42.96±17.21% of INKT cells from nine healthy blood donors (n=9) after primary stimulation with irradiated autologous PBMC negative fraction and αGalCer or 7DW8-5 and secondary expansion with irradiated allogeneic PBMCs and EBV-B cells, αGalCer or 7DW8-5, IL-2, IL-15, and/or IL-7 were CD7⁺ and CD7, respectively. Again, the majority of INKT cells (>96%) were CD2⁺ (n=7) and CD5⁺ (n=9). Therefore, we hypothesized that CD7⁻ subsets in INKT cells expressing CD7 CAR can partially avoid fratricide without nuclease (e.g., CRISPR)-mediated genome editing of CD7 knockout and be expanded in our allo culture conditions for adoptive cell therapy of a CD7⁺ cancer.

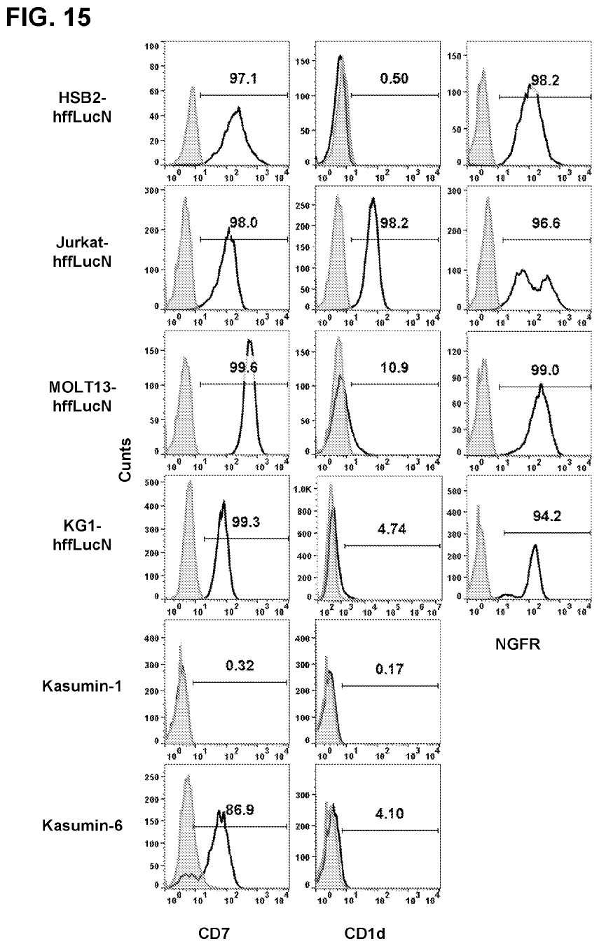

FIG. 15 shows that expression of CD7, CD1d and NGFR in lentiviral engineered human T-ALL (HSB2, Jurkat and MOLT13) and AML (KG1) cell lines expressing luciferase and NGFR was confirmed by flow cytometric analysis. Two additional AML cell lines, Kasumi-1 and Kasumi-6 were CD7⁻ and CD7⁺, respectively, and CD1d.

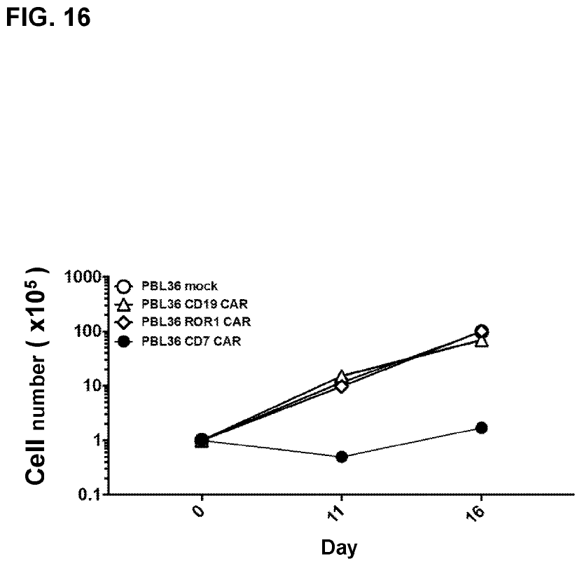

FIG. 16 shows that CD7 CAR transduced peripheral blood T cells induced substantial fratricide and failed to expand. To confirm that CD7 CAR transduced T cells induce fratricide and prevent CAR-T cell expansion, activated human T cells from a blood donor (PBL36) were transduced with a lentiviral CD7 CAR construct. CAR-T targeting CD19 or ROR1 or untransduced mock T cells were used as controls. Following transduction of T cells, there were approximately 19-30- and 41-59-fold fewer CD7 CAR-T than CD19 CAR-T or ROR1 CAR-T or mock T cells on day 11 and 16, respectively, confirming that CD7 antigen-driven fratricide precludes CD7 CAR-T expansion (Gomes-Silva et al., Blood 2017; 130 (3): 285-296; Cooper et al., Leuk. 2018; 32 (9): 1970-1983).

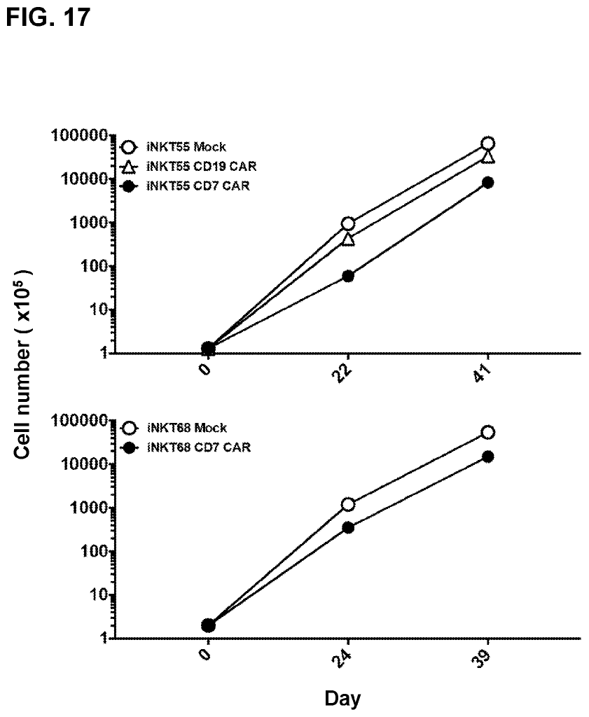

FIG. 17 shows that CD7 CAR-modified INKT cells can be expanded in culture. On day 22 and 41, 46- and 6,407-fold expansion rate was obtained in CD7 CAR-modified INKT55 cells (INKT55 CD7 CAR) whereas 334-732- and 25,661-50,492-fold expansion rate was documented in CD19 CAR-modified INKT55 (INKT55 CD19 CAR) and iNKT55 mock cells. In the end of culture, 6.5×10⁹ INKT55 mock, 3.30×10⁹ INKT55 CD19 CAR, and 8.3×10⁸ INKT55 CD7 CAR cells were generated from their starting numbers of 1.3×10⁵ INKT cells using our allo expansion protocol. In addition, CD7 CAR-modified INKT68 cells from donor 68 can also be expanded in culture. On day 24 and 39, 176- and 7,470-fold expansion rate was obtained in CD7 CAR-modified INKT68 cells (INKT68 CD7 CAR) whereas 600- and 27,120-fold expansion rate was documented in iNKT68 mock cells. In the end of culture, 5.4×10⁹ INKT68 mock and 1.5×10⁹ INKT68 CD7 CAR were generated from their starting numbers of 2×10⁵ iNKT cells. It appears that approximately 6,000-fold expansion of CD7 CAR- or CD19 CAR-modified INKT cells can be achieved after two rounds of stimulation. On average, 2.526±1.495×10⁵ INKT cells (n=27 donors) were isolated from 108 PBMCs using anti-iNKT microbeads. It is expected that about 1.5×10⁹ or 50×10⁹ CD7 CAR-modified INKT cells with 70-90% CAR⁺ can be generated from 108 PBMCs or one leukopak (containing about 5-8×10⁹ PBMCs), respectively. Thus, about 175 infusions of CD7 CAR-modified INKT cells from one leukopak could be achieved based on current Food and Drug Administration (FDA) approved Yescarta (axicabtagene ciloleucel, CD19 CAR-T) dose of 2×10⁶ CAR⁺ T cells per kg body weight, with a maximum of 2×10⁸ CAR⁺-T cells for patients with large B-cell lymphoma.

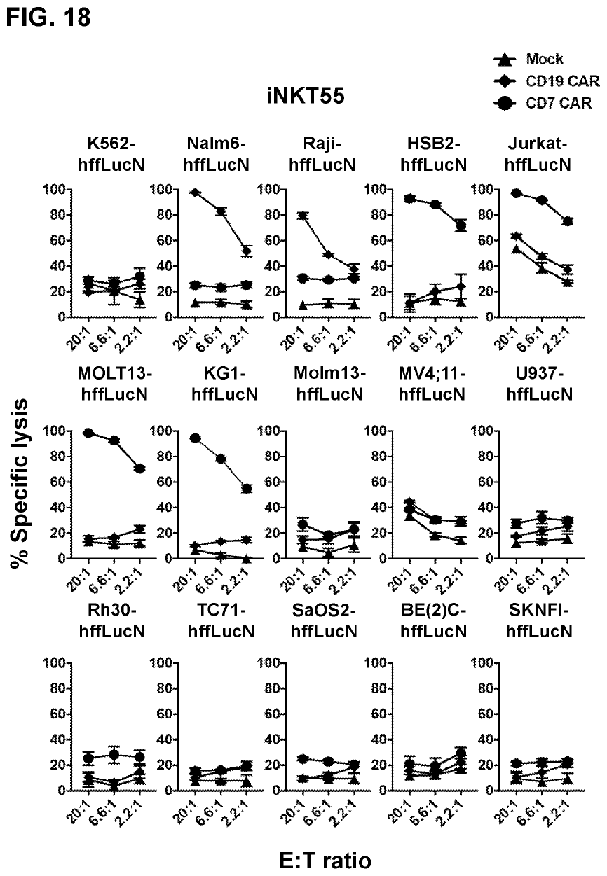

FIG. 18 shows one representative cytotoxicity plot in which CD7 CAR-modified INKT55 were able to kill luciferase-expressing CD7⁺ T-ALL (HSB2, Jurkat, MOLT13) and CD7⁺ AML (KG1) cell lines in an E/T ratio dependent manner but not CD7⁻ tumor cell lines including AML (Molm13, MV4;11, U937), soft tissue sarcoma (Rh30, TC71, SaOS2), neuroblastoma (BE (2) C, SKNFI), and CML (K562). CD7 CAR-modified iNKT55 displayed a low background cytotoxicity against B-cell leukemia and lymphoma cell lines (Nalm6, Raji) compared to mock cells. As controls, CD19 CAR-modified INKT55 specifically lysed CD19⁺ malignant B cells (Nalm6, Raji) but not CD19-T-ALL, AML, sarcoma, and neuroblastoma. Untransduced mock iNKT cells did not kill all tested target cells including CD1d⁺ AML (KG1, Molm13, MV4:11, U937) but CD1d⁺ Jurkat cells, confirming a previous study demonstrating that many human leukemic T-cells express CD1d and can be directly killed by Vα24⁺ NKT cells (Takahashi et al., Br J Haematol. 2003; 122 (2): 231-239).

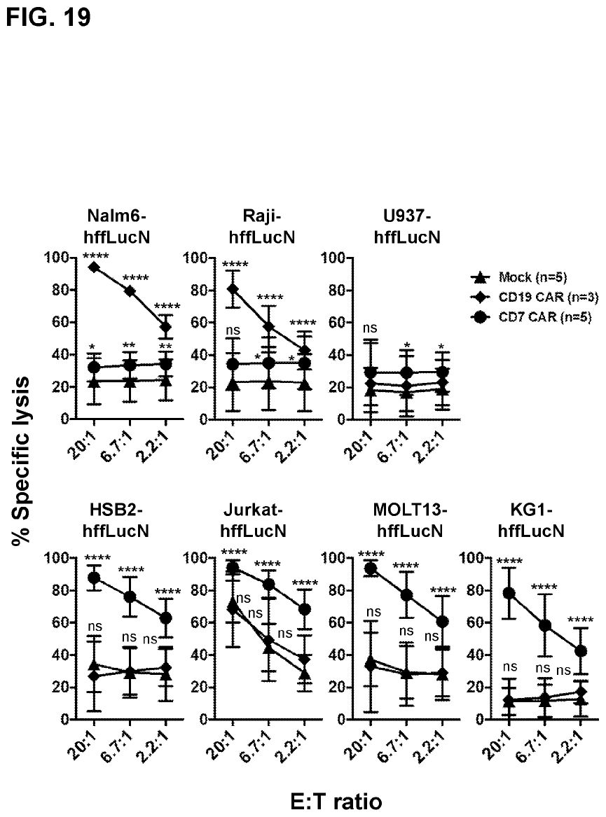

FIG. 19 shows a combined cytotoxicity plot with statistical analyses in which CD7 CAR-modified INKT cells (n=5 donors) specifically killed luciferase-expressing CD7⁺ T-ALL (HSB2, Jurkat, MOLT13) and CD7⁺ AML (KG1) at all E/T ratios (20:1, 6.7;1, and 2.2:1) compared to mock iNKT cells and CD19 CAR-modified INKT cells (p<0.0001). CD7 CAR INKT cells appeared to show a low background cytotoxicity to CD7⁻ B-cell leukemia and lymphoma cells (Nalm6, Raji) as well as U937 (at an E/T ratio of 6.7:1 and 2.2:1 but not 20:1) when compared to mock iNKT cells (n=5 donors) (p<0.05). As controls, CD19 CAR-modified INKT cells (n=3 donors) specifically killed CD19⁺ B-cell tumor cells but not CD19⁻ T-ALL and AML cells (p<0.0001). Mock INKT cells showed minimal cytotoxicity to all target cells except for CD1d⁺ Jurkat T-ALL cells.

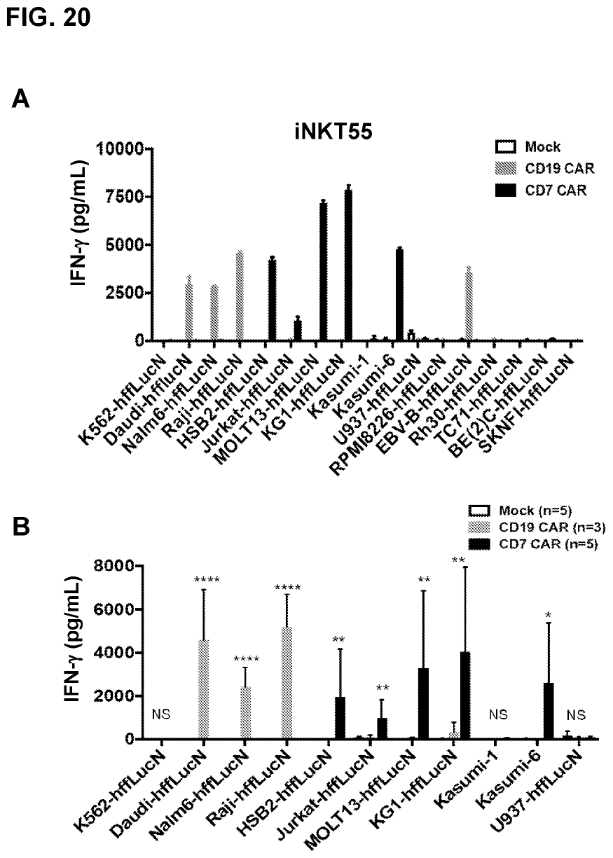

To demonstrate CD7 CAR-modified INKT cells also secrete IFN-γ in response to CD7⁺ tumor cell stimulation, co-culture supernatants were examined for IFN-γ levels by ELISA. FIG. 20A shows that CD7 CAR-modified INKT55 cells from one representative donor produced at least 4,000-fold higher amounts of IFN-γ in co-culture with CD7⁺ T-ALL (HSB2, MOLT13) and CD7⁺ AML (KG1, Kasumi-6) than CD19 CAR-modified INKT55 and mock INKT55 cells. CD7 CAR-modified INKT55 cells secreted 1,000-fold more IFN-γ to Jurkat T-ALL than CD19 CAR INKT and mock INKT cells. As expected, CD19 CAR-modified INKT cells produced at least 2,500-fold more IFN-γ in co-culture with CD19⁺ leukemia and lymphoma cells (Daudi, Nalm6, Raji) and EBV-transformed B cells (EBV-B). CD7 CAR INKT and CD19 CAR INKT cells produced minimal amounts of IFN-γ to CD7⁻ AML (Kasumi-1, U937), sarcoma (Rh30, TC71), neuroblastoma (BE (2) C, SKNFI), and CML (K562). Untransduced mock iNKT cells produced negligible amounts of IFN-γ to all tested tumor cells. It is noted that CD7 CAR INKT or mock cells expanded with irradiated allogeneic PBMCs and EBV-B, αGalCer or 7DW8-5, IL2, and IL-15 did not recognize EBV-B cells which were used for iNKT expansion, suggesting that CD7 CAR INKT cells expanded in allo conditions are CD7⁻ specific and may not be alloreactive.

To demonstrate that CD7 CAR-modified INKT cells reproducibly produce IFN-γ in an antigen-specific fashion, we examined IFN-γ production in CD7 CAR INKT cells from multiple donors (n=5). As shown in FIG. 20B, CD7 CAR-modified INKT cells from all 5 donors (n=5) produced significant amounts of IFN-γ in response to CD7⁺ T-ALL (HSB2, Jurkat, MOLT13) and CD7⁺ AML (KG1, Kasumi-6) compared to mock INKT (n=5, p<0.001) and CD19 CAR INKT cells (p<0.05 except for Jurkat, p<0.01). Control CD19 CAR-modified INKT cells (n=3) produced significant amounts of IFN-γ to CD19⁺ B-cell leukemia and lymphoma cell lines (Daudi, Nalm6, Raji) compared to mock INKT and CD7 CAR INKT cells (p<0.0001). CD7 CAR INKT and CD19 CAR INKT cells did not produce significant amounts of IFN-γ in recognition of CD7⁻ or CD19-K562 (CML), Kasumi-1 (AML) and U937 (AML). Mock iNKT showed minimal IFN-γ production to all target cells tested, even to cytotoxicity-susceptible CD1d⁺ Jurkat (T-ALL) cells (FIG. 18 and FIG. 19, Takahashi et al., Br J Haematol. 2003; 122 (2): 231-239).

FIG. 21 shows that CD7 CAR expression in transduced INKT cells (n=5) ranged from 64.6% to 98.8% whereas CD19 CAR or ROR1 CAR expression varied from 5.37% to 43.9%. It appears that CD7 CAR transduction efficiency in INKT cells was higher than CD19 CAR or ROR1 CAR as the CD7 antigen-mediated fratricide can enrich CD7 CAR⁺ INKT cell populations.

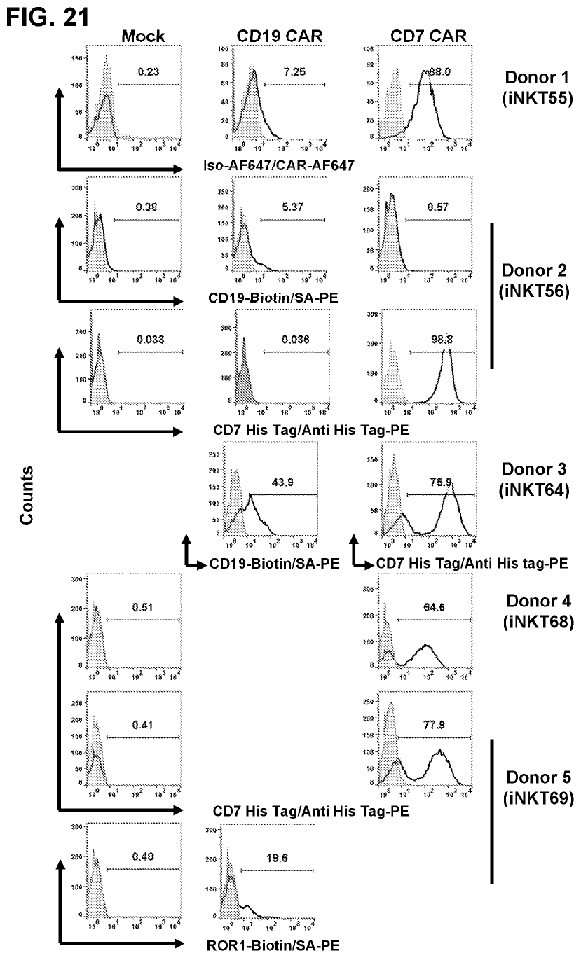

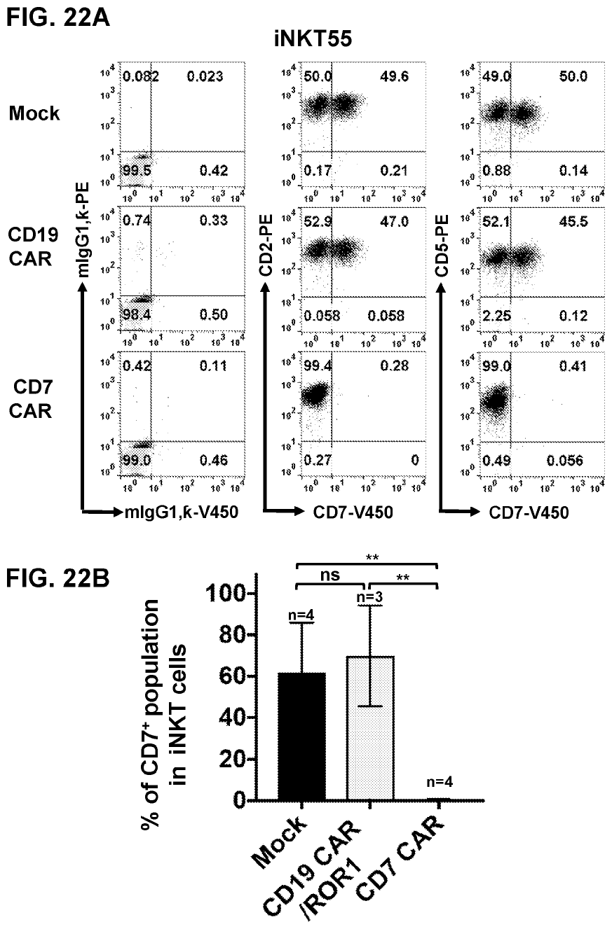

To confirm that the fratricide results in complete elimination of CD7⁺ INKT cells by CD7 CAR-modified INKT cells, a flow cytometric dot blot analysis of expression of CD2 versus CD7 and CD5 versus CD7 was performed. FIG. 22A shows the data from one representative donor 55 in which 45-50% of CD7⁺ cells in mock INKT and CD19 CAR INKT cells was eliminated to 0.4% in CD7 CAR INKT cells. FIG. 22B demonstrates that CD7 CAR INKT cells significantly eliminated almost all CD7⁺ cells (mean±SD 0.4%±0.1, n=4) compared to mock iNKT (61.6%±24.4, n=4) and CD19 or ROR1 CAR INKT cells (69.9%±24.4, n=3) (p<0.0001).

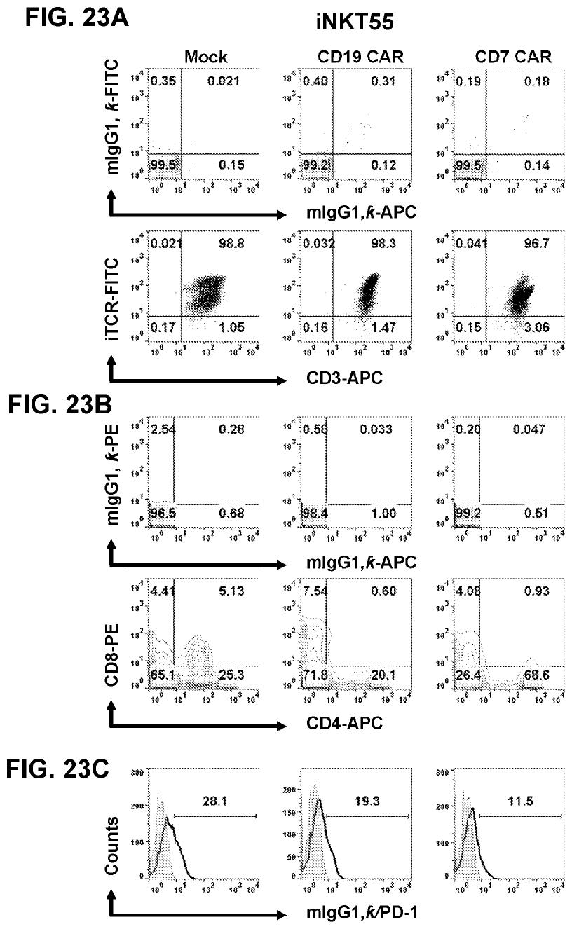

Phenotypically, expression of CD3⁺iTCR⁺ in CD7 CAR INKT cells remained unchanged compared to mock INKT and CD19 CAR INKT cells as shown in FIG. 23A. Like mock iNKT and CD19 CAR INKT cells, CD7 CAR INKT cells were composed of CD4⁺, CD4⁻CD8⁻, CD8⁺, and CD4⁺CD8⁺ populations (FIG. 23B). It appears that CD7 CAR INKT cells were differentiated to more CD4⁺ than CD8⁺ cells and expressed less cell surface PD-1 inhibitory molecules than mock or CD19 CAR INKT cells (FIG. 23B, FIG. 23C).

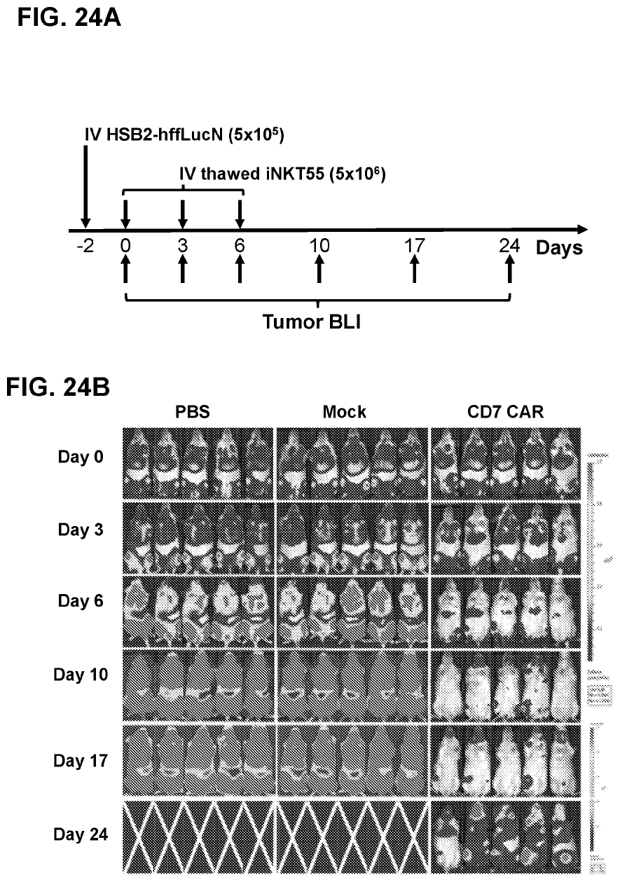

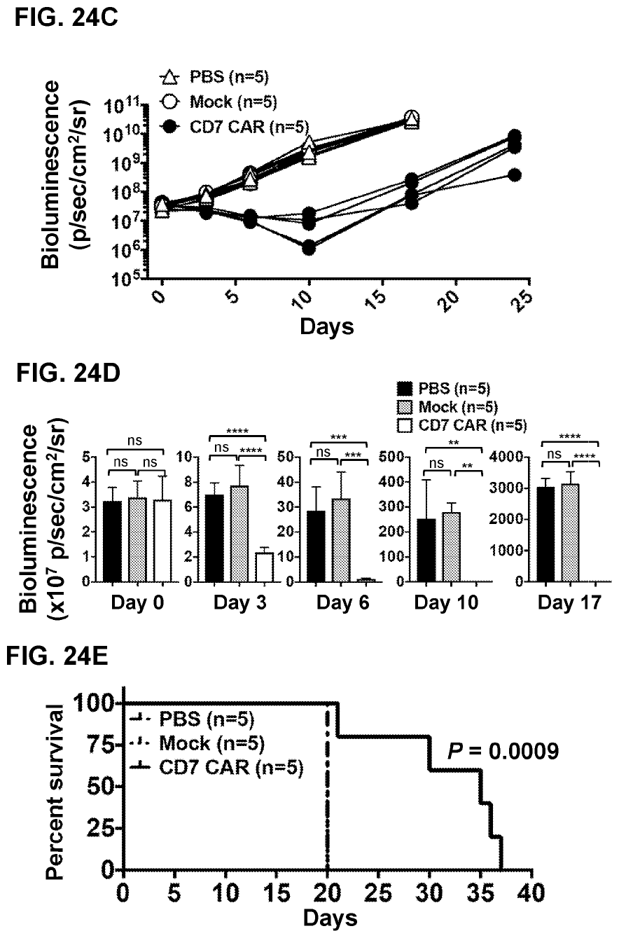

A time-dynamic bioluminescent imaging (BLI) technique in live mice was used to assess the anti-leukemic effect of CD7 CAR-modified INKT cells. For a xenograft T-ALL model, NSG mice were i.v. injected with aggressive HSB2-hfflucN T-ALL cells and randomly divided into three groups (n=5 mice per group) based on BLI readings (p=0.942). The mice were then i.v. infused with thawed CD7 CAR INKT cells, mock iNKT cells or PBS (FIG. 24A). The administration of 3 infusions of thawed CD7 CAR INKT cells into leukemia-bearing mice significantly suppressed T-ALL growth at days 3 (p<0.0001), 6 (p=0.0001-0.0005), 10 (p=0.0012-0.0024) and 17 (p<0.0001) compared to PBS and mock controls (FIG. 24B, FIG. 24C, FIG. 24D). A significant survival advantage was observed after CD7 CAR INKT cell therapy compared to PBS and mock control groups (p=0.0009, FIG. 24E).

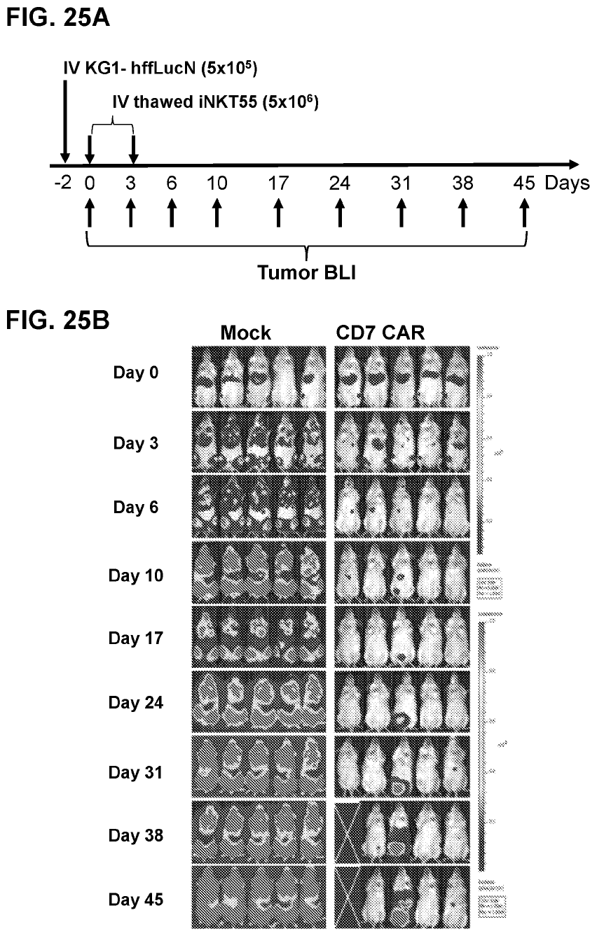

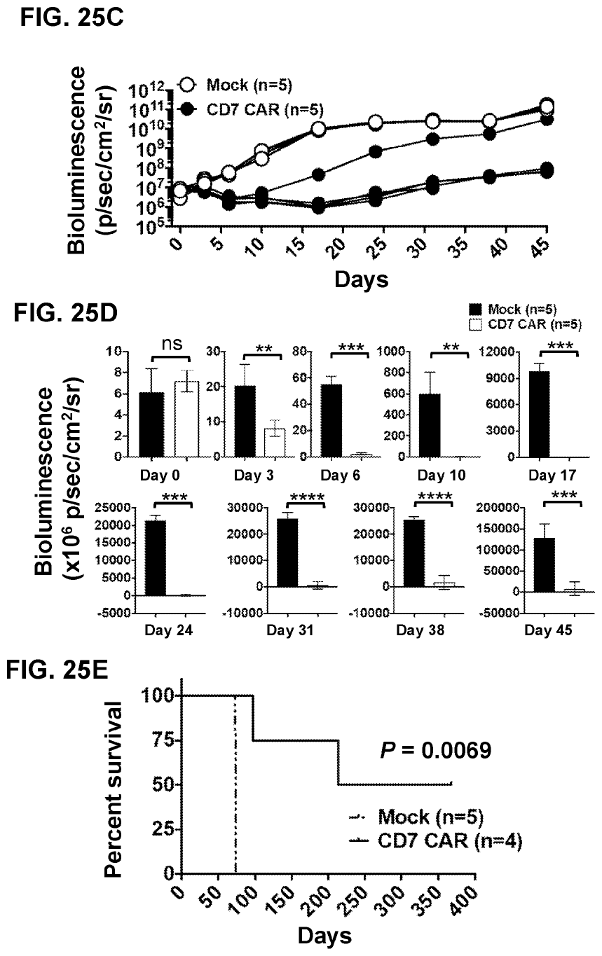

We also evaluated the anti-leukemia effect of CD7 CAR-modified INKT cells in a xenograft CD7⁺ AML model. FIG. 25A shows the experimental schedule of tumor cell injection, CD7 CAR INKT cell infusion and BLI monitoring. NSG mice were i.v. injected with KG1-hfflucN AML cells and then i.v. infused with thawed CD7 CAR INKT cells and mock INKT cells (n=5 mice per group) (FIG. 25A). Prior to treatment, no significant difference in tumor mass was found between the two groups (p=0.3787). Two infusions of thawed CD7 CAR INKT cells into pre-established AML mice significantly suppressed AML growth at days 3 (p=0.0096), 6 (p<0.0001), 10 (p=0.0026), 17 (p<0.0001), 24 (p<0.0001), 31 (p<0.0001), 38 (p<0.0001) and 45 (p=0.0003) compared to mock control (FIG. 25B, FIG. 25C, FIG. 25D). CD7 CAR INKT cell therapy also significantly prolonged the animal survival for more than one year in 50% of treated mice while mock control mice died around day 74 (p=0.0069, FIG. 25E). Altogether, these results demonstrate that CD7 CAR-modified INKT cells without genome editing and expanded in our allo culture conditions can mount anti-CD7⁺ tumor responses in vitro and in vivo.

The foregoing embodiments and examples are provided for illustrative purposes only and are not intended to limit the scope of the invention. Many variations to those described above may be possible and apparent to those of skill in this art based on the present disclosure. Such modifications and variations are within the spirit and scope of the present invention. All patent or non-patent literature cited are incorporated herein by reference in their entireties without admission of them as prior art.

1. Isolated and activated invariant natural killer T (INKT) cells or type I natural killer T (NKT) cells modified to express a CD7 specific chimeric antigen receptor (CAR) without the use of genome editing.

2. The isolated and activated invariant natural killer T (INKT) cells or type I natural killer T (NKT) cells of claim 1, wherein the cells are CD3⁺iTCR(Vα24-Jα18)⁺ or CD3⁺Vα24⁺ isolated from a biological sample and activated in the presence of α-galactosylceramide (αGalCer) or 7DW8-5 or other glycolipid analog(s); and wherein the cells are modified to express a CD7 chimeric antigen receptor (CAR) by a lentiviral vector, and expanded in culture using irradiated allogeneic peripheral blood mononuclear cells and Epstein Barr virus transformed B-cells, αGalCer or 7DW8-5, IL-2, IL-15, and/or IL-7.

3. The isolated and activated invariant natural killer T (iNKT) cells or type I natural killer T (NKT) cells of claim 1, wherein the CD3⁺iTCR(Vα24-Jα18)⁺ or CD3 Vα24⁺ iNKT cells have the phenotypes of CD3 iTCR⁺CD4⁺ cells, CD3⁺iTCR⁺CD8⁺ cells, CD3⁺iTCR⁺CD4⁻CD8⁻ cells, or CD3⁺iTCR⁺CD4⁺CD8⁺ cells, or a mixture thereof.

4. The isolated and activated invariant natural killer T (INKT) cells or type I natural killer T (NKT) cells of claim 2, wherein the biological sample is selected from blood, bone marrow, lymph node tissue, spleen tissue, tumor tissue, induced pluripotent stem cells, and peripheral blood mononuclear cells, and combinations thereof; and wherein optionally the blood is peripheral blood and/or umbilical cord blood.

5. The isolated and activated invariant natural killer T (iNKT) cells or type I natural killer T (NKT) cells of claim 1, wherein the cells are isolated from one or more peripheral blood mononuclear cells.

6. The isolated and activated invariant natural killer T (INKT) cells or type I natural killer T (NKT) cells of claim 1, wherein the cells comprise one or more polynucleotides encoding the CD7 specific CAR.

7. The isolated and activated invariant natural killer T (iNKT) cells or type I natural killer T (NKT) cells of claim 1, wherein the CD7 specific CAR comprises an antigen binding domain, a hinge domain, a transmembrane domain, and an intracellular signaling domain.

8. The isolated and activated invariant natural killer T (iNKT) cells or type I natural killer T (NKT) cells of claim 7, wherein the antigen binding domain is capable of binding to CD7.

9. The isolated and activated invariant natural killer T (iNKT) cells or type I natural killer T (NKT) cells of claim 7, wherein the antigen binding domain comprises an antibody or an antigen binding fragment thereof, or a non-antibody protein scaffold; wherein the antibody is a monoclonal antibody, a polyclonal antibody, a synthetic antibody, a human antibody, a humanized antibody, or a single domain antibody (or a nanobody); wherein the antigen binding fragment comprises a single chain variable fragment (scFv); and wherein the intracellular signaling domain comprises a functional signaling domain of at least one stimulatory molecule.

10. The isolated and activated invariant natural killer T (iNKT) cells or type I natural killer T (NKT) cells of claim 9, wherein the at least one stimulatory molecule comprises a zeta chain associated with a T cell receptor complex or a CD3 zeta chain; wherein the intracellular signaling domain further comprises a functional signaling domain of at least one costimulatory molecule; and wherein optionally the at least one costimulatory molecule is 4-1BB, CD28, CD27, CD134 (OX40), ICOS, DAP10, or DAP12.

11. The isolated and activated invariant natural killer T (INKT) cells or type I natural killer T (NKT) cells of claim 1, wherein the cells are further modified to comprise an exogenous cytokine, growth factor, antibody or antigen binding fragment, or any combination thereof, wherein the antibody or antigen binding fragment optionally comprises a bispecific T cell engager (BiTE).

12. A pharmaceutical composition comprising the isolated and activated invariant natural killer T (INKT) cells or type I natural killer T (NKT) cells according to claim 1 and a pharmaceutically acceptable carrier.

13. A method of treating or preventing a CD7⁺ cancer in a subject in need thereof,
comprising administering to the subject a therapeutically effective amount of the isolated and activated invariant natural killer T (INKT) cells or type I natural killer T (NKT) cells according to claim 1.

14. The method of claim 13, wherein the cancer is selected from a CD7⁺ hematological malignancy; T-cell lymphoblastic leukemias (T-ALL) and T-ALL subtypes including early thymic precursors (ETP)-ALL (ETP-ALL), Pro-T-ALL, Pre-T-ALL, Cortical T-ALL and Mature T-ALL; a subset of peripheral T-cell lymphomas (PTCLs) and PTCL subtypes including PTCL, not otherwise specified (PTCL-NOS), anaplastic large cell lymphoma (ALCL), primary cutaneous ALCL, angioimmunoblastic T-cell lymphoma (AITL), nasal NK/T-cell lymphoma, adult T-cell acute lymphoblastic lymphoma or leukemia (ATLL) associated with the human T-cell leukemia virus-1 (HTLV-1) infection, enteropathy-associated lymphoma, hepatosplenic lymphoma, subcutaneous panniculitis-like lymphoma, precursor T-cell acute lymphoblastic lymphoma or leukemia, blastic NK-cell lymphoma, and cutaneous T-cell lymphomas (CTCLs); CD7⁺ acute myeloid leukemia (AML); and CD7⁺ other malignancies or CD7⁺CD1d⁺ malignancies, and wherein optionally the cancer is resistant or refractory to treatment in the absence of the cells.

15. The method of claim 13, wherein the isolated and activated invariant natural killer T (INKT) cells or type I natural killer T (NKT) cells are CD3⁺iTCR(Vα24-Jα18)⁺ or CD3 Vα24⁺ iNKT cells isolated from a biological sample of the subject or a donor, activated and engineered with synthetic receptors having a phenotype of CD3⁺iTCR⁺CD4⁺ cells, CD3⁺iTCR⁺CD8⁺ cells, CD3 iTCR⁺CD4⁻CD8⁻ cells, or CD3⁺iTCR⁺CD4⁺CD8⁺ cells, or a mixture thereof.

16. (canceled)

17. The method of claim 15, wherein the cancer is a CD7⁺ hematological malignancy; T-cell lymphoblastic leukemias (T-ALL) and T-ALL subtypes including early thymic precursors (ETP)-ALL (ETP-ALL), Pro-T-ALL, Pre-T-ALL, Cortical T-ALL and Mature T-ALL; a subset of peripheral T-cell lymphomas (PTCLs) and PTCL subtypes including PTCL, not otherwise specified (PTCL-NOS), anaplastic large cell lymphoma (ALCL), primary cutaneous ALCL, angioimmunoblastic T-cell lymphoma (AITL), nasal NK/T-cell lymphoma, adult T-cell acute lymphoblastic lymphoma or leukemia (ATLL) associated with the human T-cell leukemia virus-1 (HTLV-1) infection, enteropathy-associated lymphoma, hepatosplenic lymphoma, subcutaneous panniculitis-like lymphoma, precursor T-cell acute lymphoblastic lymphoma or leukemia, blastic NK-cell lymphoma, and cutaneous T-cell lymphomas (CTCLs); CD7⁺ acute myeloid leukemia (AML); CD7⁺ other malignancies, or CD7⁺CD1d⁺ malignancies, and wherein optionally the cancer is resistant or refractory to treatment in the absence of the cells.

18. A method of preparing a therapy for treating or preventing a CD7⁺ cancer in a subject in need thereof, comprising:
a. isolating one or more invariant natural killer T cells from a biological sample based on expression of the TCRα-chain Vα24-Jα18 (iTCR) or TCRα-chain Vα24;
b. enabling or activating the one or more iTCR⁺ or Vα24⁺ invariant natural killer T cells in a growth medium for proliferation using but not limited to irradiated autologous peripheral blood mononuclear cells negative fraction and/or whole peripheral blood mononuclear cells, α-galactosylceramide (αGalCer) or 7DW8-5 or other glycolipid analog, IL-2, and IL-15 and/or IL-7;
c. lentiviral engineering activated invariant natural killer T cells to express CAR (e.g., CD7); and
d. expanding the one or more CAR (e.g., CD7) engineered invariant natural killer T cells in a growth medium using irradiated allogeneic peripheral blood mononuclear cells and Epstein Barr virus transformed B-cells, αGalCer or 7DW8-5, IL-2, and IL-15 and/or IL-7.

19. The method of claim 18, further comprising modifying the one or more invariant natural killer T cells to express a CAR or a TCR or a TCRm, wherein the modifying comprises introducing one or more polynucleotides encoding the CAR into the one or more cells; wherein introducing one or more polynucleotides optionally comprises electroporation, transduction, and/or transfection; wherein optionally the one or more polynucleotides comprise mRNA and/or DNA; and wherein optionally the DNA comprises transposon DNA.

20. The method of claim 19, wherein the one or more polynucleotides comprise one or more vectors, wherein the one or more vectors comprise one or more viral vectors or lentiviral vectors or γ-retroviral vectors.

21. The method of claim 18, wherein the cancer is a CD7⁺ hematological malignancy; T-cell lymphoblastic leukemias (T-ALL) and T-ALL subtypes including early thymic precursors (ETP)-ALL (ETP-ALL), Pro-T-ALL, Pre-T-ALL, Cortical T-ALL and Mature T-ALL; a subset of peripheral T-cell lymphomas (PTCLs) and PTCL subtypes including PTCL, not otherwise specified (PTCL-NOS), anaplastic large cell lymphoma (ALCL), primary cutaneous ALCL, angioimmunoblastic T-cell lymphoma (AITL), nasal NK/T-cell lymphoma, adult T-cell acute lymphoblastic lymphoma or leukemia (ATLL) associated with the human T-cell leukemia virus-1 (HTLV-1) infection, enteropathy-associated lymphoma, hepatosplenic lymphoma, subcutaneous panniculitis-like lymphoma, precursor T-cell acute lymphoblastic lymphoma or leukemia, blastic NK-cell lymphoma, and cutaneous T-cell lymphomas (CTCLs); CD7⁺ acute myeloid leukemia (AML); CD7⁺ other malignancies or CD7 CD1d⁺ malignancies, and wherein optionally the cancer is resistant or refractory to treatment in the absence of the cells.
